SEQUENCE LISTING (30 sequences)

SEQ ID NO: 1 - protein, 15 aa, Artificial Sequence
  Description of Artificial Sequence Synthetic peptide
GGGGSGGGGSGGGGS

SEQ ID NO: 2 - protein, 20 aa, Homo sapiens
MYRMQLLSCIALSLALVTNS

SEQ ID NO: 3 - protein, 20 aa, Mus sp.
MYSMQLASCVTLTLVLLVNS

SEQ ID NO: 4 - protein, 20 aa, Homo sapiens
METPAQLLFLLLLWLPDTTG

SEQ ID NO: 5 - protein, 20 aa, Mus sp.
METDTLLLWVLLLWVPGSTG

SEQ ID NO: 6 - protein, 21 aa, Homo sapiens
MALPVTALLLPLALLLHAARP

SEQ ID NO: 7 - protein, 18 aa, Homo sapiens
MALPVTALLLPLALLLHA

SEQ ID NO: 8 - protein, 16 aa, Homo sapiens
MKWVTFISLLFSSAYS

SEQ ID NO: 9 - protein, 30 aa, Homo sapiens
MDSKGSSQKGSRLLLLLVVSNLLLCQGVVS

SEQ ID NO: 10 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 11 - DNA, 1005 bp, Homo sapiens
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 12 - protein, 250 aa, Artificial Sequence
  Description of Artificial Sequence Synthetic polypeptide
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLLKDITSDSENSNFRNEIQSLV

SEQ ID NO: 13 - DNA, 750 bp, Artificial Sequence
  Description of Artificial Sequence Synthetic polynucleotide
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggttccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgctcaaggacattactagtgactcagaaaattcaaacttcagaaatgaaatccaaagcttggtc

SEQ ID NO: 14 - protein, 335 aa, Artificial Sequence
  Description of Artificial Sequence Synthetic polypeptide
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIVEIKNDNVQDTAEQKVQLLRNWHQLHGKKEAYDTLIKDLKKANLCTLAEKIQTIILKDITSDSENSNFRNEIQSLV

SEQ ID NO: 15 - DNA, 1005 bp, Artificial Sequence
  Description of Artificial Sequence Synthetic polynucleotide
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagttgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 16 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 17 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 18 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 19 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 20 - DNA, 81 bp, Homo sapiens
ttttgggtgctggtggtggttggtggagtcctggcttgctatagcttgctagtaacagtggcctttattattttctgggtg

SEQ ID NO: 21 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 22 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 188 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 24 - protein, 112 aa, Artificial Sequence
  Description of Artificial Sequence Synthetic polypeptide
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 26 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 27 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 28 - protein, 484 aa, Artificial Sequence
  Description of Artificial Sequence Synthetic polypeptide
ALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 1452 bp, Artificial Sequence
  Description of Artificial Sequence Synthetic polynucleotide
gctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 30 - protein, 9 aa, Artificial Sequence
  Description of Artificial Sequence Synthetic peptide
KVPRNQDWL

(19) United States
(12) Patent Application Publication
(10) Pub. No.: US 2021/0214415 A1
(43) Pub. Date: Jul. 15, 2021

(54) IMMUNORESPONSIVE CELLS EXPRESSING DOMINANT NEGATIVE FAS AND USES THEREOF

(71) Applicants: MEMORIAL SLOAN-KETTERING CANCER CENTER, New York, NY (US); THE UNITED STATES OF AMERICA, AS REPRESENTED BY THE SECRETARY, DEPARTMENT OF HEALTH AND HUMAN SERVIC, Bethesda, MD (US)

(72) Inventors: Christopher A. Klebanoff, New York, NY (US); Tori N. Yamamoto, Bethesda, MD (US); Nicholas P. Restifo, Bethesda, MD (US)

(73) Assignees: MEMORIAL SLOAN-KETTERING CANCER CENTER, New York, NY (US); THE UNITED STATES OF AMERICA, AS REPRESENTED BY THE SECRETARY, DEPARTMENT OF HEALTH AND HUMAN SERVIC, Bethesda, MD (US)

(21) Appl. No.: 17/214,436

(22) Filed: Mar. 26, 2021

Related U.S. Application Data
(63) Continuation of application No. PCT/US2019/053825, filed on Sep. 30, 2019.
(60) Provisional application No. 62/738,317, filed on Sep. 28, 2018.

(51) Int. Cl. C07K 14/705; C07K 16/28; A61P 35/00; A61P 37/04; A61K 39/00

(52) U.S. Cl. CPC C07K 14/70578; C07K 16/2809; C07K 14/70521; A61P 35/00; A61P 37/04; C07K 2319/03; A61K 39/001112; C07K 2319/02; A61K 2039/5156; C07K 2319/33; C07K 16/2803

(57) ABSTRACT

The present disclosure provides methods and compositions for enhancing the immune response toward cancers and pathogens. It relates to a cell comprising an antigen-recognizing receptor (e.g., a chimeric antigen receptor (CAR) or a T cell receptor (TCR)) and a dominant negative Fas polypeptide. In certain embodiments, the cells are antigen-directed and exhibit enhanced cell persistence, and enhanced anti-target treatment efficacy.

### CROSS-REFERENCE TO RELATED APPLICATIONS

This application is a Continuation of International Patent Application No. PCT/US19/53825 filed Sep. 30, 2019, which claims priority to U.S. Provisional Application No. 62/738,317, filed on Sep. 28, 2018, the contents of each of which are incorporated by reference in their entirety, and to each of which priority is claimed.

### GRANT INFORMATION

This invention was made with government support under grant numbers ZIA BC011586 and ZIA BC010763 awarded by the Intramural Research Programs of the NCI, Center for Cancer Research of the NIH. The government has certain rights in the invention.

### SEQUENCE LISTING

The specification further incorporates by reference the Sequence Listing submitted herewith via EFS on Mar. 26, 2021. Pursuant to 37 C.F.R. § 1.52(e)(5), the Sequence Listing text file, identified as 0727341227 SL.txt, is 42,979 bytes and was created on Mar. 26, 2021. The Sequence Listing electronically filed herewith, does not extend beyond the scope of the specification and thus does not contain new matter.

### INTRODUCTION

The presently disclosed subject matter provides methods and compositions for enhancing the immune response toward cancers and pathogens. It relates to immunoresponsive cells comprising a dominant negative Fas polypeptide. The immunoresponsive cells can further comprise an antigen-recognizing receptor (e.g., a chimeric antigen receptors (CAR) or a T cell receptors (TCR).

### BACKGROUND OF THE INVENTION

Adoptive cell immunotherapy with genetically engineered autologous or allogeneic T cells has shown evidence of therapeutic efficacy in a range of human cancers, including but not limited to melanoma and various B-cell malignancies. T cells may be modified to target tumor-associated antigens through the introduction of genes encoding artificial T-cell receptors, termed chimeric antigen receptors (CARs) or T cell receptors (TCRs), conveying specificity to antigens expressed by cancers or virally infected cells. Immunotherapy is a targeted therapy that has the potential to provide for the treatment of cancer.

Adoptive cell transfer (ACT) using genetically engineered T cells has entered the standard of care for patients with refractory B cell malignancies, including pediatric acute lymphoblastic leukemia (1) and adult aggressive B cell lymphomas (2). The exceptional efficacy of ACT in hematologic lymphoid malignancies has been consistently observed across clinical trials, regardless of institution, gene vector, or cell composition (3-8). By contrast, responses to adoptive immunotherapy in patients with solid malignancies, collectively the leading cause of adult cancer-related deaths (9), have been comparatively modest (10-13). Thus, there is still a need for new strategies that enhances the potency of transferred T cells.

### SUMMARY OF THE INVENTION

The presently disclosed subject matter provides cells (e.g., T cells, Tumor Infiltrating Lymphocytes, or Natural Killer (NK) cells) that comprise a dominant negative Fas polypeptide. In certain embodiments, the cell comprises: (a) an antigen-recognizing receptor (e.g., a CAR or a TCR) that binds to an antigen, and (b) an exogenous dominant negative Fas polypeptide. In certain embodiments, the dominant negative Fas polypeptide comprises at least one modification in a cytoplasmic death domain. In certain embodiments, the at least one modification is selected from the group consisting of mutations, deletions, or insertions. In certain embodiments, the at least one modification is in the cytoplasmic death domain of human Fas. In certain embodiments, the at least one modification in the cytoplasmic death domain prevents the binding between the dominant negative Fas polypeptide and a FADD polypeptide. In certain embodiments, the dominant negative Fas polypeptide comprises a deletion of the amino acids at positions 230-314 of a human Fas having the amino acid sequence set forth in SEQ ID NO: 10. In certain embodiments, the dominant negative Fas polypeptide comprises an amino acid sequence that is at least about 80% identical to the amino acid sequence set forth in SEQ ID NO: 12. In certain embodiments, the dominant negative Fas polypeptide has the amino acid sequence set forth in SEQ ID NO: 12.

In certain embodiments, the dominant negative Fas polypeptide comprises a point mutation at position 260 of a human Fas having the amino acid sequence set forth in SEQ ID NO: 10. In certain embodiments, the point mutation of the human Fas is D260V. In certain embodiments, the dominant negative Fas polypeptide comprises an amino acid sequence that is at least about 80% identical to the amino acid sequence set forth in SEQ ID NO: 14. In certain embodiments, the dominant negative Fas polypeptide has the amino acid sequence set forth in SEQ ID NO: 14.

In certain embodiments, the exogenous dominant negative Fas polypeptide enhances cell persistence of the immunoresponsive cell. In certain embodiments, the exogenous dominant negative Fas polypeptide reduces apoptosis or anergy of the immunoresponsive cell.

In certain embodiments, the antigen-recognizing receptor is exogenous or endogenous (e.g., native antigen specificity from T cells obtained from the peripheral blood, following in vitro sensitization and/or selection, or tumor infiltrating lymphocytes). In certain embodiments, the antigen-recognizing receptor is recombinantly expressed. In certain embodiments, the antigen-recognizing receptor is expressed from a vector.

In certain embodiments, the exogenous dominant negative Fas polypeptide is expressed from a vector.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, a B cell, a monocyte and a macrophage. In certain embodiments, the cell is a T cell. In certain embodiments, the T cell is a cytotoxic T lymphocyte (CTL), a regulatory T cell, or a Natural Killer T (NKT) cell. In certain embodiments, the immunoresponsive cell is autologous or allogeneic to the intended recipient.

In certain embodiments, the antigen is a tumor antigen or a pathogen antigen. In certain embodiments, the antigen is a tumor antigen. In certain embodiments, the tumor antigen is selected from the group consisting of CD19, MUC16, MUC1, CA1X, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD3, HER-2, hTERT, IL-13R-a2, K-light chain, KDR, mutant KRAS, mutant PIK3CA, mutant IDH, mutant p53, mutant NRAS, LeY, L1 cell adhesion molecule, MAGE-A1, Mesothelin, ERBB2, MAGEA3, CT83 (also known as KK-LC-1), p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, and CD99, CD70, ADGRE2, CCR1, LILRB2, PRAME, HPV E6 oncoprotein, HPV E7 oncoprotein, and ERBB. In certain embodiments, the tumor antigen is CD19.

In certain embodiments, the antigen is a pathogen-associated antigen. In certain embodiments, the pathogen-associated antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

In certain embodiments, the antigen-recognizing receptor is a T cell receptor (TCR) or a chimeric antigen receptor (CAR). In certain embodiments, the antigen-recognizing receptor is an endogenous TCR that recognizes a pathogen-associated antigen, and said cell is a pathogen-specific T cell. In certain embodiments, the antigen-recognizing receptor is an endogenous TCR that recognizes a tumor antigen, and said cell is a tumor-specific T cell. In certain embodiments, the antigen-recognizing receptor is a CAR. In certain embodiments, the CAR comprises an extracellular antigen-binding domain, a transmembrane domain, and an intracellular signaling domain. In certain embodiments, the CAR further comprises a co-stimulatory signaling domain. In certain embodiments, the at least one co-stimulatory signaling domain comprises a CD28 polypeptide.

In certain embodiments, the cell further comprises a suicide gene. In certain embodiments, the suicide gene is a Herpes simplex virus thymidine kinase (hsv-tk), inducible Caspase 9 Suicide gene (iCasp-9) or a truncated human epidermal growth factor receptor (EGFRt) polypeptide.

The presently disclosed subject matter provides compositions (e.g., pharmaceutical compositions) comprising an effective amount of the cells disclosed herein. In certain embodiments, the composition is a pharmaceutical composition that further comprises a pharmaceutically acceptable carrier. In certain embodiments, the composition is for treating and/or preventing a neoplasia and/or a pathogen infection.

The presently disclosed subject matter provides methods of inducing and/or enhancing an immune response to a target antigen. In certain embodiments, the method comprises administering to the subject an effective amount of the cells disclosed herein or a pharmaceutical composition comprising thereof.

The presently disclosed subject matter provides methods of reducing tumor burden in a subject. In certain embodiments, the method comprises administering to the subject an effective amount of the cells disclosed herein or a pharmaceutical composition comprising thereof. In certain embodiments, the method reduces the number of tumor cells. In certain embodiments, the method reduces tumor size. In certain embodiments, the method eradicates the tumor in the subject.

The presently disclosed subject matter provides methods of treating and/or preventing neoplasia, or lengthening survival of a subject having a neoplasia. In certain embodiments, the method comprises administering to the subject an effective amount of the cells or a pharmaceutical composition comprising thereof.

In certain embodiments, the tumor or neoplasm is selected from the group consisting of blood cancer, B cell leukemia, multiple myeloma, lymphoblastic leukemia (ALL), chronic lymphocytic leukemia, non-Hodgkin's lymphoma. myeloid leukemias, and myelodysplastic syndrome (MDS). In certain embodiments, the neoplasm is B cell leukemia, multiple myeloma, lymphoblastic leukemia (ALL), chronic lymphocytic leukemia, or non-Hodgkin's lymphoma, and the antigen is CD19. In certain embodiments, the neoplasia is selected from a solid cancer. Selected solid malignancies could include cancers originating from the brain, breast, lung, gastro-intestinal tract (including esophagus, stomach, small intestine, large intestine, and rectum), pancreas, prostate, soft tissue/bone, uterus, cervix, ovary, kidney, skin, thymus, testis, head and neck, or liver.

The presently disclosed subject matter provides methods of treating blood cancer in a subject. In certain embodiments, the method comprises administering to the subject an effective amount of T cells, wherein the T cell comprises an antigen-recognizing receptor that binds to an antigen and an exogenous dominant negative Fas polypeptide. In certain embodiments, the blood cancer is selected from the group consisting of B cell leukemia, multiple myeloma, acute lymphoblastic leukemia (ALL), chronic lymphocytic leukemia, and non-Hodgkin's lymphoma, myeloid leukemias, and myelodysplastic syndrome (MDS).

The presently disclosed subject matter provides methods of treating a solid tumor in a subject. In certain embodiments, the method comprises administering to the subject an effective amount of T cells, wherein the T cell comprises an antigen-recognizing receptor that binds to an antigen and an exogenous dominant negative Fas polypeptide. In certain embodiments, the solid tumor is selected from the group consisting of tumors originated from the brain, breast, lung, gastro-intestinal tract (including esophagus, stomach, small intestine, large intestine, and rectum), pancreas, prostate, soft tissue/bone, uterus, cervix, ovary, kidney, skin, thymus, testis, head and neck, or liver.

The presently disclosed subject matter provides methods of preventing and/or treating a pathogen infection in a subject. In certain embodiments, the method comprises administering to the subject an effective amount of the cells disclosed herein or a pharmaceutical composition comprising thereof. In certain embodiments, the pathogen is selected from the group consisting of a virus, a bacterium, a fungus, a parasite and a protozoon capable of causing disease.

The presently disclosed subject matter provides methods for producing an antigen-specific cell. In certain embodiments, the method comprises introducing into a cell (a) a first nucleic acid sequence encoding an antigen-recognizing receptor that binds to an antigen; and (b) a second nucleic sequence encoding an exogenous dominant negative Fas polypeptide. In certain embodiments, one or both of the first and second nucleic acid sequence is operably linked to a promoter element. In certain embodiments, one or both of the first and second nucleic acid sequences are comprised in a vector. In certain embodiments, the vector is a retroviral vector.

The presently disclosed subject matter provides a nucleic acid composition comprising (a) a first nucleic acid sequence encoding an antigen-recognizing receptor and (b) a second nucleic acid sequence encoding an exogenous dominant negative Fas polypeptide. In certain embodiments, one or both of (a) and (b) are operably linked to a promoter element. In certain embodiments, one or both of the first and second nucleic acid sequences are comprised in a vector. In certain embodiments, the vector is a retroviral vector.

The presently disclosed subject matter further provides a vector comprising the nucleic acid composition disclosed herein.

The presently disclosed subject matter provides a kit comprising a cell disclosed herein, a nucleic acid composition disclosed herein, or a vector disclosed herein. In certain embodiments, the kit further comprises written instructions for treating and/or preventing a neoplasia and/or or a pathogen infection.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A-1F depict that human tumor microenvironments overexpress the death-inducing ligand FASLG. (A) A pan-cancer analysis of FASLG expression within the microenvironments of 26 different tumor types relative to matched normal tissues of origin. RNA-sequencing (RNA-seq) data from human cancers and matched normal tissues was extracted from the Cancer Genome Atlas (TCGA) and Genotype-Tissue Expression datasets, analyzed using UCSC Xena, and displayed as normalized RNA-Seq by Expectation Maximization (RSEM) values. Statistical comparisons of expression between tumors and normal tissues were made using a Mann-Whitney t test with Bonferroni correction; ***P<0.001, **P<0.01, *P<0.05. (B) Selected, pre-ranked gene set enrichment analyses (GSEAs) against all KEGG pathways of genes positively correlated to FASLG expression averaged across 26 TCGA histologies. Circle diameters reflect the number of genes identified within the GSEA signature sets. The nominal P-value and FDR q value for all displayed GSEAs were <0.001. (C) Pearson's correlation of the top 200 correlated genes to FASLG gene expression across 26 human cancer types in the TCGA database. Selected immune-related genes associated with the GSEA signature sets listed in panel (B) are identified. (D,E) Representative histogram (D) and summary plot of Fas MFI (E) on phenotypically defined CD8a⁺ T cell subsets. Data shown are from peripheral blood T cells from 47 patients and HDs. CD8⁺ T cell subsets in panels (D) and (E) were defined as follows: TN cells, CD8a⁺CD45RA⁺CD45RO⁻ CCR7⁺CD62L⁺CD27⁺CD28⁺Fas⁻; TCM, CD8a⁺CD45RO⁺CD45RA⁻CCR7⁺CD62L⁺; TEM, CD8a⁺CD45RO⁺CD45RA⁻CCRTCD62L⁻; TEMRA, CD8a⁺CD45RA⁺CCRTCD62L⁻. (F) The fraction of TN among all CD8a⁺ T cells in the circulation of age-matched healthy donors (HD; n=39; left), and patients with melanoma (MEL; n=20; middle) and diffuse large B cell lymphoma (DLBCL; n=17; right) at the time of enrollment to an adoptive immunotherapy clinical trial. ***P<0.001, ns=not significant (two-way ANOVA).

FIGS. 2A-2D depict that murine T cells engineered with Fas DNRs prevent FasL-mediated apoptosis. (A) Schematic representation of physiologic Fas signaling and the design of two murine Fas dominant negative receptors (DNRs). Retroviral-encoded Fas DNRs were designed to prevent recruitment of Fas-associated protein with death domain (FADD) either by (i) substitution of an asparagine for an isoleucine residue at position 246 of the death domain (DD; Fas^{I246N}), or (ii) truncation of the majority of the intracellular death domain (Fas^{ΔDD}). Wildtype Fas (Fas^{WT}) and an empty vector were used as controls. Receptors were cloned into a bicistronic vector containing a Thy1.1 reporter. EC, extracellular domain; TM, transmembrane domain; T2A, thosea asigna virus 2A self-cleaving peptide. (B) Experimental timeline for the stimulation, retroviral transduction, expansion, and testing of lz-FasL mediated apoptosis of WT CD8α⁺ T cells modified with Fas^{I246N}, Fas^{ΔDD}, Fas^{WT}, or an empty vector control. (C) Representative FACS plots and (D) summary bar graph showing the frequency of apoptotic Annexin V⁺/PI⁺ transduced T cells at rest and 6h following exposure to lz-FasL (50 ng mL⁻¹). Results are shown after gating on transduced Thy1.1⁺ cells. Data shown is representative of 6 independently performed experiments and is displayed as mean±SEM with n=3 per condition. ***P<0.001, ns=not significant (two-way ANOVA).

FIGS. 3A-3H depict enhanced survivability of Fas DNR-engineered T cells in the tumor microenvironment. (A) Experimental schema for the generation and co-infusion of congenically distinguishable, WT pmel-1 CD8α⁺ T cells engineered with Fas^{ΔDD} DNR (Ly5.1⁺ Thy1.1⁺) or an empty vector control (Ly5.1⁻Thy1.1⁺). Transduced T cells were enriched with an anti-Thy1.1 microbead prior to recombination in a about 1:1 mixture and a total of 8e⁶ T cells were infused i.v. into sublethally irradiated (6 Gy) Thy1.1⁻Ly5.1⁻ mice bearing 10d established B16 melanoma tumors. Recipient mice received IL-2 by daily i.p. injection for 3d and the spleens and tumors were harvested for analysis on d7. (B) Relative persistence of Fas^{ΔDD} DNR-modified to empty vector-modified T cells in the spleens and tumors of recipient mice. Results displayed after gating on live, CD8α⁺ Thy1.1⁺ lymphocytes and are representative of two independent experiments, each with n=5-8 mice. ***P<0.001 (unpaired 2-tailed Student's t test). (C) Representative FACS plots and (D) summary bar graph of T-cell viability following overnight culture in cytokine-free media alone, in the presence of B16 melanoma, or with lz-FasL (50 ng T cells were transduced either with Fas^{ΔDD} DNR or empty vector control without bead enrichment prior to initiation of the overnight culture. Data shown after gating on Thy1.1⁺ and Thy 1.1⁻ lymphocytes. Bar graphs are displayed as mean±SEM and is representative of 4 independent experiments with n=3 replicates per condition. (E) Relative persistence of Fas^{ΔDD} DNR-modified to empty vector-modified T cells in the spleens and tumors of recipient mice. Results after gating on live CD8α⁺ Thy1.1⁺ lymphocytes are representative of 2 independent experiments, each with n=5-8 mice. “****P<0.0001, **P<0.01, paired 2-tailed Student's t test. (F) Total number of live Ly5.1⁺CD8eV1313⁺cells transduced with the empty or Fas^{ΔDD} construct. (G) Relative fold expansion of Fas^{ΔDD} normalized to empty construct found in spleen on the indicated days. (H) Percentage of live Ly5.1⁺CD8α⁺Vβ13⁺ cells expressing Ki-67 for each condition. Representative plots from 2 independent experiments. Data are displayed as mean±SEM with n=3 per condition. *P<0.05, Wilcoxon's rank-sum test.

FIGS. 4A-4E depict that transfer of Fas DNR-modified T cells does not result in acquired autoimmune lymphoproliferative syndrome (ALPS). (A) Representative FACS plots and (B) summary bar graph of the frequency of CD3⁺B220⁺CD4⁻CD8α⁻ double negative T cells in the spleens of WT mice who received 6 Gy sublethal irradiation followed by adoptive transfer of 5e⁵ bead-purified Thy1.1⁺pmel-1 T cells modified with Fas^{ΔDD} DNR or an empty vector control. Recipient mice also received IL-2 daily by i.p. injection for 3d. Age-matched wild type mice and Fas-deficient lpr/lpr mice served as negative and positive controls, respectively. (C) Representative FACS plots (D) and summary scatter plot demonstrating the persistence and surface phenotype of transferred pmel-1 T cells modified with Fas^{ΔDD} DNR or an empty vector control after >6 months. All data shown is representative of 5 independent experiments, each with n=5-8 mice per cohort. ***P<0.001, *P<0.05 (one-way ANOVA). (E) Experimental design to analyze long-term persistence of WT pmel-1 CD8α⁺ T cells modified with Fas^{ΔDD} or empty vector control in B6 mice.

FIGS. 5A-5H depict that adoptive transfer of Fas DNR-modified T cells enhances antitumor efficacy independently of T-cell differentiation status. (A) Experimental design for the generation of WT pmel-1 CD8⁺ T cells modified with Fas^{ΔDD}, Fas^{I246N}, or empty vector control. (B) Tumor regression and (C) survival of mice bearing 10d established B16 melanoma tumors who were left untreated as controls or received 5×10⁵ bead-purified Thy1.1⁺pmel-1 cells modified with Fas^{ΔDD}, Fas^{I246N}, or empty vector control. All treated mice received sublethal irradiation (6 Gy) prior to cell infusion followed by 3d of i.p. IL-2. (D) Representative FACS plots demonstrating the purity of sorted CD62L⁺CD44⁺ Thy1.1⁺ TCM-like pmel-1 T cells modified with Fas DNRs or empty vector control prior to infusion. (E) Tumor regression and (F) survival of mice bearing 10d established B16 melanoma tumors who were untreated or received 5×10⁵ of sort-purified TCM-like Thy1.1⁺ modified cells. (G) Tumor regression and (H) survival of mice bearing 10-day-established B16 melanoma tumors that were untreated or received 5×10⁵ of sort-purified T_{CM}-like Thy1.1⁺ modified cells. All tumor measurements were performed in a blinded fashion by an independent investigator. Representative results from two independent experiments are shown as mean±SEM using n=5-8 mice/cohort. Statistical comparisons performed using Wilcoxon rank sum test (B, E, G) or the Log-rank Mantel Cox test (C, F, H). **P<0.01; *P<0.05.

FIGS. 6A-6D depict that genetic engineering with Fas DNR protects human T cells from FasL-induced apoptosis. (A) Schematic representation of physiologic Fas signaling and the design of two human Fas dominant negative receptors (DNRs). Retroviral-encoded human Fas DNRs were designed to prevent recruitment of Fas-associated protein with death domain (FADD) either by (i) substitution of a valine for an aspartic acid residue at position 260 of the death domain (DD; hFas^{D260V}), or (ii) truncation of the majority of the human intracellular death domain (hFas^{ΔDD}; ΔDD=deletion of aa 230-314 of human Fas). An empty vector was used as a negative control. Receptors were cloned into a bicistronic vector containing a Thy1.1 reporter. EC, extracellular domain; TM, transmembrane domain; T2A, a 2A self-cleaving peptide derived from Thosea asigna virus 2A. (B) Experimental timeline for the stimulation, retroviral transduction, expansion, and testing of lz-FasL mediated apoptosis of human CD8⁺ T cells derived from peripheral blood mononuclear cells (PBMCs) modified with Fas^{D244V}, Fas^{ΔDD}, or an empty vector control. (C) Representative FACS plots and (D) summary graph showing the frequency of apoptotic Annexin V⁺ T cells at rest and 6h following exposure to titrated concentrations of lz-FasL. Results shown after gating on transduced (Thy1.1⁺) or untransduced (Thy1.1⁻) T cells. Data is displayed as mean±SEM with n=3 per condition displayed and is representative of 3 independent experiments. *P<0.05, ns=not significant (Wilcoxon rank sum test).

FIGS. 7A-7D depict design and expression of retrovirally-encoded murine Fas DNR constructs and controls in mouse CD8⁺ T cells. (A) Schematic overview of the designs for retroviral constructs encoding murine wildtype (WT) Fas or mutant versions of Fas impaired in their ability to bind the intracellular adapter molecule Fas-associated via death domain. WT Fas, Fas with an asparagine replacing the isoleucine at position 246 (Fas^{I246N}), or Fas with truncation of the intracellular death domain (Fas^{ΔDD}) were cloned into an MSGV1 expression vector in front of a T2A cleavage site and the Thy1.1 reporter gene. An empty vector containing only the Thy1.1 reporter gene (Empty) was used as a negative control. (B) Representative FACS plots and summary bar graphs of (C) Thy1.1 and (D) Fas expression 4d following retroviral transduction of Fas-deficient lpr/lpr or WT CD8α⁺ T cells. The percentage of gated Thy1.1⁺ or Fas⁺ cells is shown in black, MFI of Thy1.1⁺ or Fas⁺ cells is shown in red on flow plots. Data in (C) and (D) are displayed as mean±SEM with n=3 per condition and is representative of 12 independent experiments.

FIGS. 8A-8D depict that Fas DNRs prevent lz-FasL induced AKT activation and T-cell differentiation. (A, B) Representative FACS histograms (top) and summary plot (bottom) of the dose-response relationship between lz-FasL exposure and (A) phospho-AKT^{S473} and (B) phospho-S6^{S235/236} in CD8α⁺ T cells transduced with Fas^{I246N}, Fas^{ΔDD}, or empty vector control. Results shown 6d after activation, retroviral transduction, and expansion in the continuous presence of indicated concentrations of lz-FasL. (C) Representative FACS plots of T-cell differentiation (top) and intracellular IFNγ/IL-2 production (bottom) 11d after CD8α⁺ T cells were transduced Fas^{I246N}, Fas^{ΔDD}, or empty vector control in the absence of exogenous FasL. Intracellular cytokine staining measured after ˜5 hr incubation with PMA/ionomycin in brefeldin A and monensin. (D) Memory T cell subset composition of CD8α⁺ T cells 11d after activation, transduction, and expansion in culture. Graphs displayed as mean±SEM with n=3 per condition and is representative of 3 (A, B) and 5 (C, D) independent experiments. *P<0.05, (Wilcoxon rank sum test).

FIGS. 9A-9E depict the effects of Fas DNR and anti-CD19 CAR modified T cell treatment in a mouse model of leukemia. (A) Experimental design for the treatment with syngeneic T cells co-transduced with anti-CD19 CAR and either Fas^{ΔDD} or or an empty vector control in a mouse leukemia model. All treated mice received sublethal irradiation (5 Gy) prior to cell infusion followed by 3d of i.p. IL-2. (B) Co-transduction efficiency and (C) Representative FACS plots demonstrating the purity of sorted Thy1.1⁺ T cells modified with anti-CD19 CAR and either Fas^{ΔDD} or empty vector control. (D) Survival of mice bearing 10d established E2a:PBX pre-B ALL tumors who were left untreated as controls or received high CART cell dose (5.5×10⁵) of sort-purified Thy1.1⁺ T cells modified with anti-CD19 CAR and either Fas^{ΔDD} or empty vector control. (E) Survival of mice bearing 10d established E2a:PBX pre-B ALL tumors who were left untreated or received low CAR T cell dose (1.8×10⁵) of sort-purified Thy1.1⁺ T cells modified with anti-CD19 CAR and either Fas^{ΔDD} or empty vector control. All tumor measurements were performed in a blinded fashion by an independent investigator.

FIGS. 10A-10G show that the expression of Fas DNR enhances antiapoptotic functions and in vivo persistence in anti-CD19 CAR model. (A) Representative flow plots and (B) summary data of double transduction of B6 CD8α⁺ T cells with retroviral constructs encoding anti-CD19 CAR and empty or Fas DNR. Analysis performed on day 11 after Thy1.1 bead enrichment on day 6. (C) Summary bar graph of relative T cell viability (to Fas^{ΔDD}) following overnight culture in cytokine-free media alone, with lz-FasL (100 ng ml⁻¹), 2 μg ml⁻¹ each of anti-CD3 and anti-CD28, or E2a-PBX. Data shown after gating on Thy1.1⁺ lymphocytes are representative of 3 independently performed experiments, and displayed as mean±SEM with n=3 per condition. *P<0.05, ****P<0.0001, 2-way ANOVA. (D) Experimental schema for the generation and infusion of WT CD8α⁺ T cells engineered to express anti-CD19 CAR along with Fas^{ΔDD} DNR or an empty vector control. Transduced T cells were Thy1.1 bead enriched prior to injection, and T cells were infused i.v. into sublethally irradiated (5 Gy) mice bearing 4-day-established E2a-PBX leukemia. Spleens and BM were harvested for analysis on day 14. co-Td, cotransduced. (E) Summary data of numbers of live CD8α⁺ Thy1.1⁺ lymphocytes in spleens and BM of recipient mice. (F) Summary data of the frequency of E2a-PBX leukemia in the BM of recipient mice. Results in E and F are representative of 2 independent experiments, each with n=3-5 mice. *P<0.05, **P<0.01, ****P<0.0001, 1-way ANOVA, corrected with Tukey's multiple comparisons. (G) Survival of mice bearing 4-day-established E2a-PBX leukemia that were untreated or received 3×10⁵ (left) or 2×10⁵ (right) anti-CD19 CAR⁺ Thy1.1⁺ modified cells. Representative results from 4 independent experiments are shown as mean±SEM using n=5 mice/cohort. Statistical comparisons were performed using the log-rank Mantel-Cox test; *P<0.05 **P<0.01.

FIG. 11 depicts Fas DNRs can protect non-transduced cells from FasL-mediated apoptosis. Summary bar graph showing the relative frequency of cell viability of non-transduced and transduced T cells after 20h following exposure to lz-FasL (100 ng mL⁻¹). Results shown after gating on live CD8α⁺lymphocytes, and viability shown relative to the media for each transduction condition. Data shown is representative of 3 independently performed experiments and is displayed as mean±SEM with n=3 per condition. ****P<0.0001, ns=not significant (one-way ANOVA, corrected with Tukey's multiple comparisons).

FIGS. 12A-12D illustrate the expression of Fas^{I246N} in T cells does not cause reversion to WT Fas. (A) Experimental timeline for the stimulation, retroviral transduction, and analysis of WT CD8α⁺ T cells modified with Fas^{WT} or Fas^{I246N}. (B) Representative FACS plots of Thy1.1 expression at days 6 and 12 for Fas^{WT} or Fas^{I246N} transduced cells. (C) Experimental timeline for the stimulation, transduction, Thy1.1-enrichment, and sequencing of WT CD8α⁺ T cells modified with Fas^{WT} or Fas^{I246N}. (D) Representative sequencing data showing WT Fas maintains the A-T-C sequence encoding the isoleucine at amino acid position 246, whereas the Fas^{I246N} sequence is A-A-C, encoding an asparagine at amino acid position 246 in the introduced Fas DNR construct.

FIG. 13 depicts IFNγ upregulating FasL on surface of B16 tumor cells. B16 cells were treated with vehicle (PBS) or IFNγ (100 ng mL⁻¹) for 24 hours, then analyzed for surface expression of MHC Class I (H-2Db; left panel) or FasL (right panel) by flow cytometry.

FIG. 14 illustrates T cells engineered with Fas DNRs preventing apoptosis from various stimuli. Summary bar graph showing the relative frequency of cell viability of transduced T cells after 20h following exposure to lz-FasL (100 ng mL⁻¹). Results are shown after gating on Thy1.1⁺ cells, and viability is shown relative to Fas^{ΔDD}. Data shown is representative of 10 independently performed experiments and is displayed as mean±SEM with n=3 per condition. *P<0.05 **P<0.01 ****P<0.0001, ns=not significant (one-way ANOVA, corrected with Tukey's multiple comparisons).

FIG. 15A-15H show that Fas DNR expression does not induce lymphoproliferation in the ALPS-susceptible MRL strain. (A) Schematic comparing the onset of lymphoproliferation in C57BL/6 B6-lpr mice at 6-9 months (top) to the MRL-lpr strain at 3-4 months. (B) Experimental design to analyze long-term persistence of WT anti-CD19 CAR expressing CD8α⁺ T cells modified with FasADD or empty vector control in WT MRL-Mp mice. A total of 3×10⁶ of anti-CD19 CAR′ CD8α⁺ T cells were infused i.v. into sublethally irradiated (6 Gy XRT) mice. Recipient mice received IL-2 by daily i.p. injection for 3d and the spleens were harvested for analysis after 93d. (C) Summary numbers of spleen weight in recipient mice, compared to age-matched wild type mice and Fas-deficient B6-lpr mice (negative and positive controls, respectively). (D, E) Representative FACS plots (D) and (E) summary bar graph of the frequency of CD3⁺B220⁺ double negative lymphocytes in the spleens of recipient and control mice. (F) Summary bar graphs of levels of anti-nuclear antibody (ANA) Ig (top) and anti-dsDNA Ig (bottom) as measured by ELISA. (G, H) Summary bar graphs demonstrating the persistence (G) and surface phenotype (H) of transferred Thy1.1⁺ T cells modified with Fas^{ΔDD} DNR or an empty vector control. n=27 mice per cohort. ****P<0.0001, ***P<0.001, **P<0.01, *P<0.05, ns=not significant (one-way ANOVA, corrected with Tukey's multiple comparisons).

FIGS. 16A-16B depict adoptively transferred T cell modified with Fas DNR do not induce an inflammatory infiltrate in the lungs of ALPS-susceptible MRL host mice. (A) Representative H&E stained micrographs and (B) summary graph demonstrating the intensity of inflammatory mononuclear cell infiltrates in the lungs of treated mice. The arrow and star point to areas of dense peri-vascular and peri-bronchiolar mononuclear inflammatory infiltrates, respectively. Scale bar=300 μm. All images were scored in a blinded fashion by an interpreting pathologist. ***P<0.001, ns=not significant (one-way ANOVA, corrected with Tukey's multiple comparisons).

FIGS. 17A-17E show genetic co-engineering of primary human T cells with a Fas dominant negative receptor (ADD), antigen-specific TCR (NY-ESO-1, 1G4) and a trackable suicide switch (truncated EGFR). (A) Design of human retroviral constructs used in these experiments. (B) Schematic diagram of primary human T cell co-modified with a TCR and Fas^{DNR}. (C) Co-expression of the human Fas^{DNR} and the tEGFR suicide switch. (D) Antigen-specific cytokine production and (E) response to lz-FasL.

FIGS. 18A-18D depict genetic co-engineering of primary human T cells with a Fas dominant negative receptor (ADD), antigen-specific CAR (anti-CD19, 28z) and a trackable suicide switch (truncated EGFR). (A) Design of human retroviral constructs used in these experiments. (B) Schematic diagram of primary human T cell co-modified with a CAR and Fas^{DNR}. (C) Time-dependent induction of apoptosis in human T cells modified with tEGFR alone or combination with the hFas^{DNR} following lz-FasL exposure. (D) Antigen-specific cytokine release and degranulation in human T cells modified with an anti-CD19 CAR alone or in combination with the hFas^{DNR}.

### DETAILED DESCRIPTION OF THE INVENTION

The presently disclosed subject matter provides cells, including genetically modified immunoresponsive cells (e.g., T cells or NK cells) comprising a dominant negative Fas polypeptide. In certain embodiments, the immunoresponsive cell further comprises an antigen-recognizing receptor (e.g., a TCR or a CAR). The presently disclosed subject matter also provides methods of using such cells for inducing and/or enhancing an immune response to a target antigen, and/or treating and/or preventing a neoplasm, a pathogen infection, or other diseases/disorders (e.g., a disease/disorder where an increase in an antigen-specific immune response is desired). The presently disclosed subject matter is based, at least in part, on the discovery that a dominant negative Fas polypeptide enhances the cell persistence, prevents activation induced cell death, prevents FasL-induced cell death, and/or improves the anti-tumor effect of an immunoresponsive cell.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

By “immunoresponsive cell” is meant a cell that functions in an immune response or a progenitor, or progeny thereof.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen. Non-limiting examples of antigen-recognizing receptors include native or endogenous T cell receptors (“TCRs”), and chimeric antigen receptors (“CARs”).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S.

Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (VH) and light chains (VL) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich i glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imuno12009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immuno11997 17(5-6): 427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” include any nucleic acid molecule that encodes a polypeptide of interest (e.g., a dominant negative Fas polypeptide) or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, conservative substitutions include substitutions within the following groups: glycine, alanine; valine, isoleucine, leucine; aspartic acid, glutamic acid, asparagine, glutamine; serine, threonine; lysine, arginine; and phenylalanine, tyrosine. In certain embodiments, one or more amino acid residues within or outside a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence outside a CDR region or a CDR region are altered.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and NBLAST) can be used.

Furthermore, sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasia, and pathogen infection of cell.

An “effective amount” (or, “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

By “enforcing tolerance” is meant preventing the activity of self-reactive cells or immunoresponsive cells that target transplanted organs or tissues.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “isolated cell” is meant a cell that is separated from the molecular and/or cellular components that naturally accompany the cell.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple VH and VL domains). In certain embodiments, the linker comprises a sequence set forth in GGGGSGGGGSGGGGS [SEQ ID NO: 1].

By “neoplasm” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVTNS [SEQ ID NO: 2] (human), MYSMQLASCVTLTLVLLVNS [SEQ ID NO: 3] (mouse); the kappa leader sequence: METPAQLLFLLLLWLPDTTG [SEQ ID NO: 4] (human), METDTLLLWVLLLWVPGSTG [SEQ ID NO: 5] (mouse); the CD8 leader sequence: MALPVTALLLPLALLLHAARP [SEQ ID NO: 6] (human); the truncated human CD8 signal peptide: MALPVTALLLPLALLLHA [SEQ ID NO: 7] (human); the albumin signal sequence: MKWVTFISLLFSSAYS [SEQ ID NO: 8] (human); and the prolactin signal sequence: MDSKGSSQKGSRLLLLLVVSNLLLCQGVVS [SEQ ID NO: 9] (human). By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-IS neoplastic cell. In certain embodiments, a tumor antigen includes any polypeptide expressed by a tumor that is capable of activating or inducing an immune response via an antigen-recognizing receptor (e.g., CD19, MUC-16) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 2. Dominant Negative Fas Polypeptide

Fas cell surface death receptor (Fas) is also known as APT1; CD95; FAS1; APO-1; FASTM; ALPS1A; TNFRSF6. GenBank ID: 355 (human), 14102 (mouse), 246097 (rat), 282488 (cattle), 486469 (dog). The protein product of Fas includes, but is not limited to, NCBI Reference Sequences NP_000034.1, NP_001307548.1, NP_690610.1 and NP_690611.1.

Fas is a member of the TNF-receptor superfamily and contains a death domain. It is involved in the regulation of programmed cell death, and has been implicated in the pathogenesis of various malignancies and diseases of the immune system. The interaction of Fas with its ligand allows the formation of a death-inducing signaling complex with other components, e.g., Fas-associated protein with death domain (FADD), which can induce programmed cell death.

In certain embodiments, a Fas polypeptide is a human Fas polypeptide. In certain embodiments, a human Fas polypeptide comprises or has the amino acid sequence of NCBI Reference No.: NP_000034.1 (SEQ ID NO: 10), which is provided below. In certain embodiments, a human Fas polypeptide comprises or has an amino acid sequence that is at least about 80%, at least about 85%, at least about 90%, at least about 95%, at least about 99%, or at least about 100% homologous or identical to the sequence set forth in SEQ ID NO: 10.

| (SEQ ID NO: 10) | |
|---|---|
| 1 | MLGIWTLLPL VLTSVARLSS KSVNAQVTDI NSKGLELRKT VTTVETQNLE GLHHDGQFCH |
| 61 | KPCPPGERKA RDCTVNGDEP DCVPCQEGKE YTDKAHFSSK CRRCRLCDEG HGLEVEINCT |
| 121 | RTQNTKCRCK PNFFCNSTVC EHCDPCTKCE HGIIKECTLT SNTKCKEEGS RSNLGWLCLL |
| 181 | LLPIPLIVWV KRKEVQKTCR KHRKENQGSH ESPTLNPETV AINLSDVDLS KYITTIAGVM |
| 241 | TLSQVKGFVR KNGVNEAKID EIKNDNVQDT AEQKVQLLRN WHQLHGKKEA YDTLIKDLKK |
| 301 | ANLCTLAEKI QTIILKDITS DSENSNFRNE IQSLV |

An exemplary nucleotide sequence encoding the amino acid sequence of SEQ ID NO: 10 is set forth in SEQ ID NO: 11, which is provided below.

(SEQ ID NO: 11)
ATGCTGGGCATCTGGACCCTCCTACCTCTGGTTCTTACGTCTGTTGCTA
GATTATCGTCCAAAAGTGTTAATGCCCAAGTGACTGACATCAACTCCAA
GGGATTGGAATTGAGGAAGACTGTTACTACAGTTGAGACTCAGAACTTG
GAAGGCCTGCATCATGATGGCCAATTCTGCCATAAGCCCTGTCCTCCAG
GTGAAAGGAAAGCTAGGGACTGCACAGTCAATGGGGATGAACCAGACTG
CGTGCCCTGCCAAGAAGGGAAGGAGTACACAGACAAAGCCCATTTTTCT
TCCAAATGCAGAAGATGTAGATTGTGTGATGAAGGACATGGCTTAGAAG
TGGAAATAAACTGCACCCGGACCCAGAATACCAAGTGCAGATGTAAACC
AAACTTTTTTTGTAACTCTACTGTATGTGAACACTGTGACCCTTGCACC
AAATGTGAACATGGAATCATCAAGGAATGCACACTCACCAGCAACACCA
AGTGCAAAGAGGAAGGATCCAGATCTAACTTGGGGTGGCTTTGTCTTCT
TCTTTTGCCAATTCCACTAATTGTTTGGGTGAAGAGAAAGGAAGTACAG
AAAACATGCAGAAAGCACAGAAAGGAAAACCAAGGTTCTCATGAATCTC
CAACCTTAAATCCTGAAACAGTGGCAATAAATTTATCTGATGTTGACTT
GAGTAAATATATCACCACTATTGCTGGAGTCATGACACTAAGTCAAGTT
AAAGGCTTTGTTCGAAAGAATGGTGTCAATGAAGCCAAAATAGATGAGA
TCAAGAATGACAATGTCCAAGACACAGCAGAACAGAAAGTTCAACTGCT
TCGTAATTGGCATCAACTTCATGGAAAGAAAGAAGCGTATGACACATTG
ATTAAAGATCTCAAAAAAGCCAATCTTTGTACTCTTGCAGAGAAAATTC
AGACTATCATCCTCAAGGACATTACTAGTGACTCAGAAAATTCAAACTT
CAGAAATGAAATCCAAAGCTTGGTC

In certain embodiments, the term “dominant negative Fas polypeptide” refers to the dominant negative form of a Fas polypeptide, which is a gene product of a dominant negative mutation of a Fas gene. In certain embodiments, a dominant negative mutation (also called “antimorphic mutations”) has an altered gene product that acts antagonistically to the wild-type allele. In certain embodiments, a dominant negative Fas polypeptide adversely affects the normal, wild-type Fas polypeptide within the same cell. In certain embodiments, the dominant negative Fas polypeptide interacts with a wild-type Fas polypeptide, but blocks its signal transduction to downstream molecules, e.g., FADD.

In certain non-limiting embodiments, the dominant negative Fas polypeptide comprises a heterologous signal peptide, for example, an IL-2 signal peptide, a kappa leader sequence, a CD8 leader sequence or a peptide with essentially equivalent activity.

In certain embodiments, the dominant negative Fas polypeptide comprises at least one modification in the intracellular domain. In certain embodiments, the at least one modification prevents the binding of Fas to a FADD polypeptide. In certain embodiments, the at least one modification is within the death domain. In certain embodiments, the at least one modification is within amino acids about 200 to about 320 of SEQ ID NO: 10. In certain embodiments, the at least one modification is within amino acids about 200 to about 319 of SEQ ID NO: 10. In certain embodiments, the at least one modification is within amino acids about 202 to about 319 of SEQ ID NO: 10. In certain embodiments, the at least one modification is within amino acids about 226 to about 319 of SEQ ID NO: 10. Death domains of Fas protein are disclosed in Tartaglia L A et al. Cell. (1993); 74(5):845-53; Itoh and Nagata. J Biol Chem. (1993); 268(15):10932; Boldin M P et al. J Biol Chem. (1995); 270(14):7795-8; and Huang B et al. Nature (1996); 384(6610):638-41, all of which are incorporated by reference herein.

In certain embodiments, the modification is selected from the group consisting of mutations, deletions, and insertions. In certain embodiments, the mutation is a point mutation.

In certain embodiments, the modification is a deletion. In certain embodiments, the dominant negative Fas polypeptide comprises a partial or complete deletion of the death domain. In certain embodiments, the dominant negative Fas polypeptide comprises or has a deletion of amino acid residues 230-314 of a human wild-type Fas polypeptide (e.g., one having the amino acid sequence set forth in SEQ ID NO: 10). In certain embodiments, the dominant negative Fas polypeptide having the deletion of amino acid residues 230-314 of a human wild-type Fas polypeptide having the amino acid sequence set forth in SEQ ID NO: 10 is designated as “hFas^{ΔDD}.” hFas^{ΔDD} has the amino acid sequence set forth in SEQ ID NO: 12. SEQ ID NO: 12 is provided below.

(SEQ ID NO: 12)
MLGIWTLLPLVLTSVARLSSKSVNAQVTDINSKGLELRKTVTTVETQNL
EGLHHDGQFCHKPCPPGERKARDCTVNGDEPDCVPCQEGKEYTDKAHES
SKCRRCRLCDEGHGLEVEINCTRTQNTKCRCKPNFECNSTVCEHCDPCT
KCEHGIIKECTLTSNTKCKEEGSRSNLGWLCLLLLPIPLIVWVKRKEVQ
KTCRKHRKENQGSHESPTLNPETVAINLSDVDLLKDITSDSENSNFRNE
ICSLV

An exemplary nucleotide sequence encoding the amino acid sequence of SEQ ID NO: 12 is set forth in SEQ ID NO: 13, which is provided below.

(SEQ ID NO: 13)
ATGCTGGGCATCTGGACCCTCCTACCTCTGGTTCTTACGTCTGTTGCTA
GATTATCGTCCAAAAGTGTTAATGCCCAAGTGACTGACATCAACTCCAA
GGGATTGGAATTGAGGAAGACTGTTACTACAGTTGAGACTCAGAACTTG
GAAGGCCTGCATCATGATGGCCAATTCTGCCATAAGCCCTGTCCTCCAG
GTGAAAGGAAAGCTAGGGACTGCACAGTCAATGGGGATGAACCAGACTG
CGTGCCCTGCCAAGAAGGGAAGGAGTACACAGACAAAGCCCATTTTTCT
TCCAAATGCAGAAGATGTAGATTGTGTGATGAAGGACATGGCTTAGAAG
TGGAAATAAACTGCACCCGGACCCAGAATACCAAGTGCAGATGTAAACC
AAACTTTTTTTGTAACTCTACTGTATGTGAACACTGTGACCCTTGCACC
AAATGTGAACATGGAATCATCAAGGAATGCACACTCACCAGCAACACCA
AGTGCAAAGAGGAAGGTTCCAGATCTAACTTGGGGTGGCTTTGTCTTCT
TCTTTTGCCAATTCCACTAATTGTTTGGGTGAAGAGAAAGGAAGTACAG
AAAACATGCAGAAAGCACAGAAAGGAAAACCAAGGTTCTCATGAATCTC
CAACCTTAAATCCTGAAACAGTGGCAATAAATTTATCTGATGTTGACTT
GCTCAAGGACATTACTAGTGACTCAGAAAATTCAAACTTCAGAAATGAA
ATCCAAAGCTTGGTC

In certain embodiments, the dominant negative Fas polypeptide comprises or has an amino acid sequence that is at least about 80%, at least about 85%, at least about 90%, at least about 95%, at least about 99%, or at least about 100% homologous or identical to the amino acid sequence set forth in SEQ ID NO: 12. In certain embodiments, the dominant negative Fas polypeptide having an amino acid sequence that is at least about 80%, at least about 85%, at least about 90%, at least about 95%, at least about 99%, or at least about 100% homologous or identical to the amino acid sequence set forth in SEQ ID NO: 12 comprises or has deletion of amino acid residues 230-314 of a human Fas polypeptide (e.g., one having the amino acid sequence set forth in SEQ ID NO: 10).

In certain embodiments, the modification is a point mutation. In certain embodiments, the dominant negative Fas polypeptide comprises or has a point mutation at position 260 of a human Fas polypeptide (e.g., one having the amino acid sequence set forth in SEQ ID NO: 10). In certain embodiments, the point mutation is D260V. In certain embodiments, the dominant negative Fas polypeptide having the point mutation D260V of a human wild-type Fas polypeptide is designated as “hFas^{D260V}” hFas^{D260V} has the amino acid sequence set forth in SEQ ID NO: 14. SEQ ID NO: 14 is provided below.

(SEQ ID NO: 14)
MLGIWTLLPLVLTSVARLSSKSVNAQVTDINSKGLELRKTVTTVETQNL
EGLHHDGQFCHKPCPPGERKARDCTVNGDEPDCVPCQEGKEYTDKAHFS
SKCRRCRLCDEGHGLEVEINCTRTQNTKCRCKPNFFCNSTVCEHCDPCT
KCEHGIIKECTLTSNTKCKEEGSRSNLGWLCLLLLPIPLIVWVKRKEVQ
KTCRKHRKENQGSHESPTLNPETVAINLSDVDLSKYITTIAGVMTLSQV
KGFVRKNGVNEAKIVEIKNDNVQDTAEQKVQLLRNWHQLHGKKEAYDTL
IKDLKKANLCTLAEKIQTIILKDITSDSENSNFRNEIOSLV

An exemplary nucleotide sequence encoding the amino acid sequence of SEQ ID NO: 14 is set forth in SEQ ID NO: 15, which is provided below.

(SEQ ID NO: 15)
ATGCTGGGCATCTGGACCCTCCTACCTCTGGTTCTTACGTCTGTTGCTA
GATTATCGTCCAAAAGTGTTAATGCCCAAGTGACTGACATCAACTCCAA
GGGATTGGAATTGAGGAAGACTGTTACTACAGTTGAGACTCAGAACTTG
GAAGGCCTGCATCATGATGGCCAATTCTGCCATAAGCCCTGTCCTCCAG
GTGAAAGGAAAGCTAGGGACTGCACAGTCAATGGGGATGAACCAGACTG
CGTGCCCTGCCAAGAAGGGAAGGAGTACACAGACAAAGCCCATTTTTCT
TCCAAATGCAGAAGATGTAGATTGTGTGATGAAGGACATGGCTTAGAAG
TGGAAATAAACTGCACCCGGACCCAGAATACCAAGTGCAGATGTAAACC
AAACTTTTTTTGTAACTCTACTGTATGTGAACACTGTGACCCTTGCACC
AAATGTGAACATGGAATCATCAAGGAATGCACACTCACCAGCAACACCA
AGTGCAAAGAGGAAGGATCCAGATCTAACTTGGGGTGGCTTTGTCTTCT
TCTTTTGCCAATTCCACTAATTGTTTGGGTGAAGAGAAAGGAAGTACAG
AAAACATGCAGAAAGCACAGAAAGGAAAACCAAGGTTCTCATGAATCTC
CAACCTTAAATCCTGAAACAGTGGCAATAAATTTATCTGATGTTGACTT
GAGTAAATATATCACCACTATTGCTGGAGTCATGACACTAAGTCAAGTT
AAAGGCTTTGTTCGAAAGAATGGTGTCAATGAAGCCAAAATAGTTGAGA
TCAAGAATGACAATGTCCAAGACACAGCAGAACAGAAAGTTCAACTGCT
TCGTAATTGGCATCAACTTCATGGAAAGAAAGAAGCGTATGACACATTG
ATTAAAGATCTCAAAAAAGCCAATCTTTGTACTCTTGCAGAGAAAATTC
AGACTATCATCCTCAAGGACATTACTAGTGACTCAGAAAATTCAAACTT
CAGAAATGAAATCCAAAGCTTGGTC

In certain embodiments, the dominant negative Fas polypeptide comprises or has an amino acid sequence that is at least about 80%, at least about 85%, at least about 90%, at least about 95%, at least about 99%, or at least about 100% homologous or identical to the amino acid sequence set forth in SEQ ID NO: 14. In certain embodiments, the dominant negative Fas polypeptide having an amino acid sequence that is at least about 80%, at least about 85%, at least about 90%, at least about 95%, at least about 99%, or at least about 100% homologous or identical to the amino acid sequence set forth in SEQ ID NO: 14 comprises or has the point mutation D260V of a human Fas polypeptide (e.g., one having the amino acid sequence set forth in SEQ ID NO: 10).

In certain non-limiting embodiments, the dominant negative Fas polypeptide comprises a heterologous signal peptide, for example, an IL-2 signal peptide, a kappa leader sequence, a CD8 leader sequence or a peptide with essentially equivalent activity.

### 3. Antigen-Recognizing Receptors

The present disclosure provides antigen-recognizing receptors that bind to an antigen. In certain embodiments, the antigen-recognizing receptor is a chimeric antigen receptor (CAR). In certain embodiments, the antigen-recognizing receptor is a T-cell receptor (TCR). The antigen-recognizing receptor can bind to a tumor antigen or a pathogen antigen.

### 3.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include

CD19, MUC16, MUC1, CA1X, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD3, HER-2, hTERT, IL-13R-a2, K-light chain, KDR, mutant KRAS (including, but not limited to, G12V, G12D, G12C), mutant PIK3CA (including, but not limited to, E52K, E545K, H1047R, H1047L), mutant IDH (including, but not limited to, R132H), mutant p53 (including, but not limited to, R175H, Y220C, G245D, G245S, R248L, R248Q, R248W, R249S, R273C, R273L, R273H and R282W), mutant NRAS (including, but not limited to, Q61K), LeY, L1 cell adhesion molecule, MAGE-A1, Mesothelin, ERBB2, MAGEA3, CT83 (also known as KK-LC-1), p53, MART1,GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, and CD99, CD70, ADGRE2, CCR1, LILRB2, PRAME, HPV E6 oncoprotein, HPV E7 oncoprotein, and ERBB. In certain embodiments, the tumor antigen is CD19.

In certain embodiments, the antigen-recognizing receptor binds to a human CD19 polypeptide. In certain embodiments, the human CD19 polypeptide comprises the amino acid sequence set forth in SEQ ID NO: 16, which is provided below.

[SEQ ID NO: 16]
PEEPLVVKVEEGDNAVLQCLKGTSDGPTQQLTWSRESPLKPFLKLSLGL
PGLGIHMRPLAIWLFIFNVSQQMGGFYLCQPGPPSEKAWQPGWTVNVEG
SGELFRWNVSDLGGLGCGLKNRSSEGPSSPSGKLMSPKLYVWAKDRPEI
WEGEPPCLPPRDSLNQSLSQDLTMAPGSTLWLSCGVPPDSVSRGPLSWT
HVHPKGPKSLLSLELKDDRPARDMWVMETGLLLPRATAQDAGKYYCHRG
NLTMSFHLEITARPVLWHWLLRTGGWK

In certain embodiments, the antigen-recognizing receptor binds to the extracellular domain of a human CD19 protein.

In certain embodiments, the antigen-recognizing receptor binds to a pathogen antigen, e.g., for use in treating and/or preventing a pathogen infection, for example, in an immunocompromised subject. Non-limiting examples of pathogens include a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses), human papilloma virus (i.e. HPV), JC virus, Epstein Bar Virus, Merkel cell polyoma virus.

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtherias, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Clostridium difficile, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 3.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247ζ/ζ or ζ/η. When a TCR complex engages with its antigen and WIC (peptide/WIC), the T cell expressing the TCR complex is activated.

In certain embodiments, the TCR is an endogenous TCR. In certain embodiments, the TCR recognizes a viral antigen. In certain embodiments, the TCR is expressed in a virus-specific T cell. In certain embodiments, the virus-specific T cell is derived from an individual immune to a viral infection, e.g., BK virus, human herpesvirus 6, Epstein-Barr virus(EBV), cytomegalovirus or adenovirus. In certain embodiments, the virus-specific T cell is a T cell disclosed in Leen et al., Blood, Vol. 121, No. 26, 2013; Barker et al., Blood, Vol. 116, No. 23, 2010; Tzannou et al., Journal of Clinical Oncology, Vol. 35, No. 31, 2017; or Bollard et al., Blood, Vol. 32, No. 8, 2014, each of which is incorporated by reference in its entirety. In certain embodiments, the TCR recognizes a tumor antigen. In certain embodiments, the TCR is expressed in a tumor-specific T cell. In certain embodiments, the tumor-specific T cell is a tumor-infiltrating T cell generated by culturing T cells with explants of a tumor, e.g., melanoma or an ephithelial cancer. In certain embodiments, the tumor-specific T cell is a T cell disclosed in Stevanovic et al, Science, 356, 200-205, 2017; Dudley et al. Journal of Immunotherapy, 26(4): 332-342, 2003; or Goff et al, Journal of Clinical Oncology, Vol. 34, No. 20, 2016, each of which is incorporated by reference in its entirety.

In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 3.3. Chimeric Antigen Receptor (CAR)

In certain embodiments, the antigen-recognizing receptor is a CAR. CARs are engineered receptors, which graft or confer a specificity of interest onto an immune effector cell or immunoresponsive cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3t chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3). “Third generation” CARs comprise those that provide multiple co-stimulation (e.g., CD28 and 4-1BB) and activation (CD3). In certain embodiments, the antigen-recognizing receptor is a first generation CAR.

In certain non-limiting embodiments, the extracellular antigen-binding domain of the CAR (embodied, for example, an scFv or an analog thereof) binds to an antigen with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸M or less, about 1×10⁻⁸ M or less, about 9×10⁻⁹ M or less, about 5×10⁻⁹ M or less, about 4×10⁻⁹M or less, about 3×10⁻⁹ or less, about 2×10⁻⁹ M or less, or about 1×10⁻⁹ M or less. In certain non-limiting embodiments, the K_{d} is about 3×10⁻⁹M or less. In certain non-limiting embodiments, the K_{d} is from about 1×10⁻⁹ M to about 3×10⁻⁷ M. In certain non-limiting embodiments, the K_{d} is from about 1.5×10⁻⁹M to about 3×10⁻⁷ M. In certain non-limiting embodiments, the K_{d} is from about 1.5×10⁻⁹ M to about 2.7×10⁻⁷ M.

Binding of the extracellular antigen-binding domain (for example, in an scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet).

In accordance with the presently disclosed subject matter, a CAR comprises an extracellular antigen-binding domain, a transmembrane domain and an intracellular signaling domain, wherein the extracellular antigen-binding domain specifically binds to an antigen, which can be a tumor antigen or a pathogen antigen.

In certain embodiments, the CAR comprises an extracellular antigen-binding domain that binds to CD19. In certain embodiments, the CAR is one described in Kochenderder, J N et al. Blood. 2010 Nov. 11; 116(19):3875-86, which is incorporated by reference in its entirety.

3.3.1. Extracellular Antigen Binding Domain of A CAR

In certain embodiments, the extracellular antigen-binding domain specifically binds to an antigen. In certain embodiments, the antigen is a tumor antigen. In certain embodiments, the tumor antigen is CD19. In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the extracellular antigen-binding domain is a Fab, which is optionally crosslinked. In certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen.

3.3.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3t polypeptide, a CD4 polypeptide, a 4-1BB polypeptide, an OX40 polypeptide, an ICOS polypeptide, a synthetic peptide (not based on a protein associated with the immune response), or a combination thereof.

In certain embodiments, the transmembrane domain comprises a CD8 polypeptide. In certain embodiments, the CD8 polypeptide comprises or has an amino acid sequence that is at least about 85%, about 90%, about 95%, about 96%, about 97%, about 98%, about 99% or about 100% homologous or identical to the sequence having a NCBI Reference No: NP_001139345.1 (SEQ ID NO: 17) (homology herein may be determined using standard software such as BLAST or FASTA), or fragments thereof, and/or may optionally comprise up to one or up to two or up to three conservative amino acid substitutions. In certain embodiments, the CD8 polypeptide comprises or has an amino acid sequence that is a consecutive portion of SEQ ID NO: 17 which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 137 to 209 150 to 200, or 200 to 235 of SEQ ID NO: 17. In certain embodiments, the CAR comprises a transmembrane domain of CD8 (e.g., human CD8) or a portion thereof. In certain embodiments, the CAR of the presently disclosed comprises a transmembrane domain comprising a CD8 polypeptide comprising or having an amino acid sequence of amino acids 137 to 209 of SEQ ID NO: 17. SEQ ID NO: 17 is provided below.

[SEQ ID NO: 17]
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV

In certain embodiments, the CD8 polypeptide comprises or has an amino acid sequence that is at least about 85%, about 90%, about 95%, about 96%, about 97%, about 98%, about 99% or about 100% homologous or identical to the sequence having a NCBI Reference No: AAA92533.1 (SEQ ID NO: 18) (homology herein may be determined using standard software such as BLAST or FASTA), or fragments thereof, and/or may optionally comprise up to one or up to two or up to three conservative amino acid substitutions. In certain embodiments, the CD8 polypeptide comprises or has an amino acid sequence that is a consecutive portion of SEQ ID NO: 18 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 18. In certain embodiments, the CAR comprises a transmembrane domain of CD8 (e.g., mouse CD8) or a portion thereof. In certain embodiments, the CAR of the presently disclosed comprises a transmembrane domain comprising a CD8 polypeptide comprising or having an amino acid sequence of amino acids 151 to 219 of SEQ ID NO: 18. SEQ ID NO: 18 is provided below.

| [SEQ ID NO: 18] | |
|---|---|
| 1 | MASPLTRELS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In accordance with the presently disclosed subject matter, a “CD8 nucleic acid molecule” refers to a polynucleotide encoding a CD8 polypeptide.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous or identical to the sequence having a NCBI Reference No: NP_006130 (SEQ ID No: 19), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 19 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, 153 to 179, or 200 to 220 of SEQ ID NO: 19. In certain embodiments, the CD28 polypeptide comprises or has an amino acid sequence of amino acids 114 to 220 of SEQ ID NO: 19. In certain embodiments, the CAR comprises a transmembrane domain of CD28 (e.g., human CD28) or a portion thereof. In certain embodiments, the CAR comprises a CD28 polypeptide comprising or having amino acids 153 to 179 of SEQ ID NO: 19. SEQ ID NO: 19 is provided below:

| [SEQ ID NO: 19] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

An exemplary nucleic acid sequence encoding amino acids 153 to 179 of SEQ ID NO: 19 is set forth in SEQ ID NO: 20, which is provided below.

[SEQ ID NO: 20]
TTTTGGGTGCTGGTGGTGGTTGGTGGAGTCCTGGCTTGCTATAGCTTGC
TAGTAACAGTGGCCTTTATTATTTTCTGGGTG

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous or identical to the sequence having a NCBI Reference No: NP_031668.3 (SEQ ID No: 21), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 21 which is at least 20, or at least 30, or at least 40, or at least 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 114 to 220, 150 to 200, 151 to 177, or 200 to 220 of SEQ ID NO: 21. In certain embodiments, the CD28 polypeptide comprises or has an amino acid sequence of amino acids 114 to 220 of SEQ ID NO: 21. In certain embodiments, the CAR comprises a transmembrane domain of CD28 (e.g., mouse CD28) or a portion thereof. In certain embodiments, the CAR comprises a CD28 polypeptide comprising or having amino acids 151 to 177 of SEQ ID NO: 21. SEQ ID NO: 21 is provided below:

| [SEQ ID NO: 21] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In accordance with the presently disclosed subject matter, a “CD28 nucleic acid molecule” refers to a polynucleotide encoding a CD28 polypeptide.

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3, a portion of a CD28 polypeptide (e.g., a portion of SEQ ID NO: 19 or SEQ ID NO: 21), a portion of a CD8 polypeptide (e.g., a portion of SEQ ID NO: 17, or a portion of SEQ ID NO: 18), a variation of any of the foregoing which is at least about 80%, at least about 85%, at least about 90%, or at least about 95% homologous or identical thereto, or a synthetic spacer sequence.

3.3.3. Intracellular Signaling Domain of a CAR

In certain non-limiting embodiments, the intracellular signaling domain of the CAR comprises a CD3ζ polypeptide, which can activate or stimulate a cell (e.g., a cell of the lymphoid lineage, e.g., a T cell). Wild type (“native”) CD3 comprises three immunoreceptor tyrosine-based activation motifs (“ITAMs”) (e.g., ITAM1, ITAM2 and ITAM3), and transmits an activation signal to the cell (e.g., a cell of the lymphoid lineage, e.g., a T cell) after antigen is bound. The intracellular signaling domain of the native CD3-chain is the primary transmitter of signals from endogenous TCRs.

In certain embodiments, the intracellular signaling domain of the CAR comprises a native CD3ζ polypeptide. In certain embodiments, the CD3ζ polypeptide comprises or has an amino acid sequence that is at least about 85%, about 90%, about 95%, about 96%, about 97%, about 98%, about 99% or about 100% homologous or identical to the sequence having a NCBI Reference No: NP_932170 (SEQ ID No: 22), or fragments thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 22, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 100 to 150, 52 or 164, or 150 to 164 of SEQ ID NO: 22. In certain non-limiting embodiments, the intracellular signaling domain of the CAR comprises a CD3ζ polypeptide having amino acids 52 to 164 of SEQ ID NO: 22. SEQ ID NO: 22 is provided below:

| [SEQ ID NO: 22] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the CD3ζ polypeptide comprises or has an amino acid sequence that is at least about 85%, about 90%, about 95%, about 96%, about 97%, about 98%, about 99% or about 100% homologous or identical to the sequence having a NCBI Reference No: NP_001106864.2 (SEQ ID No: 23), or fragments thereof, and/or may optionally comprise up to one or up to two or up to three conservative amino acid substitutions. In certain non-limiting embodiments, the CD3 polypeptide comprises or has an amino acid sequence that is a consecutive portion of SEQ ID NO: 23, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 52 to 142, 100 to 150, or 150 to 188 of SEQ ID NO: 23. SEQ ID NO: 23 is provided below:

| [SEQ ID NO: 23] | |
|---|---|
| 1 | MKWKVSVLAC ILHVRFPGAE AQSFGLLDPK LCYLLDGILF IYGVIITALY LRAKFSRSAE |
| 61 | TAANLQDPNQ LYNELNLGRR EEYDVLEKKR ARDPEMGGKQ RRRNPQEGVY NALQKDKMAE |
| 121 | AYSEIGTKGE RRRGKGHDGL YQDSHFQAVQ FGNRREREGS ELTRTLGLRA RPKACRHKKP |
| 181 | LSLPAAVS |

In certain non-limiting embodiments, the intracellular signaling domain of the CAR comprises a CD3ζ polypeptide comprising or having the amino acid sequence set forth in SEQ ID NO: 24. SEQ ID NO: 24 is provided below.

[SEQ ID NO: 24]
RVKFSRSAEPPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the intracellular signaling domain of the CAR comprises a murine CD3ζ polypeptide. In certain embodiments, the intracellular signaling domain of the CAR comprises a human CD3ζ polypeptide.

In certain non-limiting embodiments, an intracellular signaling domain of the CAR does not comprise a co-stimulatory signaling region, i.e., the CAR is a first generation CAR.

In certain non-limiting embodiments, an intracellular signaling domain of the CAR further comprises at least a co-stimulatory signaling region. In certain embodiments, the co-stimulatory region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR′ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide (e.g., an intracellular domain of CD28 or a portion thereof). In certain embodiments, the intracellular signaling domain of the CAR comprises a co-stimulatory signaling region that comprises a CD28 polypeptide (e.g., an intracellular domain of human CD28 or a portion thereof). In certain embodiments, the CD28 polypeptide comprises or has an amino acid sequence that is at least about 85%, about 90%, about 95%, about 96%, about 97%, about 98%, about 99% or 100% homologous or identical to the amino acid sequence set forth in SEQ ID NO: 19, or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 19 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, 181 to 220, or 200 to 220 of SEQ ID NO: 19. In certain embodiments, the CD28 polypeptide comprises or has an amino acid sequence of amino acids 181 to 220 of SEQ ID NO: 19.

In certain embodiments, the intracellular signaling domain of the CAR comprises a co-stimulatory signaling region that comprises a CD28 polypeptide (e.g., an intracellular domain of mouse CD28 or a portion thereof). In certain embodiments, the CD28 polypeptide comprises or has an amino acid sequence that is at least about 85%, about 90%, about 95%, about 96%, about 97%, about 98%, about 99% or about 100% homologous or identical to the amino acid sequence set forth in SEQ ID NO: 21), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 21 which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 114 to 218, 115 to 218, 150 to 200, 178 to 218, or 200 to 218 of SEQ ID NO: 21. In certain embodiments, the CD28 polypeptide comprises or has an amino acid sequence of amino acids 115 to 218 of SEQ ID NO: 21.

In accordance with the presently disclosed subject matter, a “CD28 nucleic acid molecule” refers to a polynucleotide encoding a CD28 polypeptide.

In certain embodiments, the intracellular signaling domain of the CAR comprises a murine intracellular signaling domain of CD28. In certain embodiments, the intracellular signaling domain of the CAR comprises a human intracellular signaling domain of CD28.

In certain embodiments, the intracellular signaling domain of the CAR comprises a co-stimulatory signaling region that comprises two co-stimulatory molecules: CD28 and 4-1BB or CD28 and OX40.

In certain embodiments, the intracellular signaling domain of the CAR comprises a co-stimulatory signaling region that comprises a 4-1BB polypeptide. In certain embodiments, the intracellular signaling domain of the CAR comprises a co-stimulatory signaling region that comprises an intracellular domain of 4-1BB or a portion thereof. In certain embodiments, the intracellular signaling domain of the CAR comprises a co-stimulatory signaling region that comprises an intracellular domain of human 4-1BB or a portion thereof 4-1BB can act as a tumor necrosis factor (TNF) ligand and have stimulatory activity. In certain embodiments, the 4-1BB polypeptide comprises or has an amino acid sequence that is at least about 85%, about 90%, about 95%, about 96%, about 97%, about 98%, about 99% or about 100% homologous or identical to the sequence having a NCBI Reference No: NP_001552 (SEQ ID NO: 25) or fragments thereof, and/or may optionally comprise up to one or up to two or up to three conservative amino acid substitutions. In non-limiting certain embodiments, the 4-1BB polypeptide comprises or has an amino acid sequence that is a consecutive portion of SEQ ID NO: 25 which is at least about 20, or at least about 30, or at least about 40, or at least about 50, and up to 255 amino acids in length. Alternatively or additionally, in non-limiting various embodiments, the 4-1BB polypeptide comprises or has an amino acid sequence of amino acids 1 to 255, 1 to 50, 50 to 100, 100 to 150, 150 to 200, 214-255 or 200 to 255 of SEQ ID NO: 25. In certain embodiments, the 4-1BB polypeptide comprises or has an amino acid sequence of amino acids 214-255 of SEQ ID NO: 24. SEQ ID NO: 25 is provided below:

| [SEQ ID NO: 25] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide.

In certain embodiments, the intracellular signaling domain of the CAR comprises an intracellular signaling domain of human 4-1BB or a portion thereof. In certain embodiments, the intracellular signaling domain of the CAR comprises an intracellular signaling domain of mouse 4-1BB or a portion thereof.

In certain embodiments, the intracellular signaling domain of the CAR comprises a co-stimulatory signaling region that comprises an OX40 polypeptide. In certain embodiments, the intracellular signaling domain of the CAR comprises a co-stimulatory signaling region that comprises an intracellular domain of OX40 or a portion thereof. In certain embodiments, the intracellular signaling domain of the CAR comprises a co-stimulatory signaling region that comprises an intracellular domain of human OX40 or a portion thereof. In certain embodiments, the OX40 polypeptide comprises or has an amino acid sequence that is at least about 85%, about 90%, about 95%, about 96%, about 97%, about 98%, about 99% or about 100% homologous or identical to the sequence having a NCBI Reference No: NP_003318 (SEQ ID NO: 26), or fragments thereof, and/or may optionally comprise up to one or up to two or up to three conservative amino acid substitutions. In non-limiting certain embodiments, the OX40 polypeptide comprises or has an amino acid sequence that is a consecutive portion of SEQ ID NO: 26 which is at least about 20, or at least about 30, or at least about 40, or at least about 50, and up to 277 amino acids in length. Alternatively or additionally, in non-limiting various embodiments, the 4-1BB polypeptide comprises or has an amino acid sequence of amino acids 1 to 277, 1 to 50, 50 to 100, 100 to 150, 150 to 200, or 200 to 277 of SEQ ID NO: 26. SEQ ID NO: 26 is provided below:

| [SEQ ID NO: 26] | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

In certain embodiments, the intracellular signaling domain of the CAR comprises a co-stimulatory signaling region that comprises an ICOS polypeptide. In certain embodiments, the intracellular signaling domain of the CAR comprises a co-stimulatory signaling region that comprises an intracellular domain of ICOS or a portion thereof. In certain embodiments, the intracellular signaling domain of the CAR comprises a co-stimulatory signaling region that comprises an intracellular domain of human ICOS or a portion thereof. In certain embodiments, the ICOS polypeptide comprises or has an amino acid sequence that is at least about 85%, about 90%, about 95%, about 96%, about 97%, about 98%, about 99% or about 100% homologous or identical to the sequence having a NCBI Reference No: NP_036224 (SEQ ID NO: 27) or fragments thereof, and/or may optionally comprise up to one or up to two or up to three conservative amino acid substitutions. In non-limiting certain embodiments, the ICOS polypeptide comprises or has an amino acid sequence that is a consecutive portion of SEQ ID NO: 27 which is at least about 20, or at least about 30, or at least about 40, or at least about 50, and up to 199 amino acids in length. Alternatively or additionally, in non-limiting various embodiments, the ICOS polypeptide comprises or has an amino acid sequence of amino acids 1 to 277, 1 to 50, 50 to 100, 100 to 150, or 150 to 199 of SEQ ID NO: 27. SEQ ID NO: 27 is provided below:

| [SEQ ID NO: 27] | |
|---|---|
| 1 | MKSGLWYFFL FCLRIKVLTG EINGSANYEM FIFHNGGVQI LCKYPDIVQQ FKMQLLKGGQ |
| 61 | ILCDLIKTKG SGNTVSIKSL KFCHSQLSNN SVSFFLYNLD HSHANYYFCN LSIFDPPPFK |
| 121 | VTLIGGYLHI YESQLCCQLK FWLPIGCAAF VVVCILGCIL ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

3.3.4. Exemplary CARs

In certain embodiments, a presently disclosed CAR comprises an extracellular antigen-binding domain that binds to a CD19 polypeptide (e.g., a human CD19 polypeptide), a transmembrane domain comprising a CD28 polypeptide (e.g., a transmembrane domain of human CD28 or a portion thereof), an intracellular signaling domain comprising a CD3ζ polypeptide and a co-stimulatory signaling domain comprising a CD28 polypeptide (e.g., an intracellular domain of human CD28 or a portion thereof). In certain embodiments, the CAR is designated as “CD1928ζ”. In certain embodiments, the CAR (e.g., CD1928ζ) comprises the amino acid sequence is set forth in SEQ ID NO: 28. SEQ ID NO: 28 is provided below.

[SEQ ID NO: 28]
ALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYM
QLSGLTSEDSAVYFCARKTISSVVDFYFDYWGQGTTVTVSSGGGGSGGG
GSGGGGSDIELTQSPKFMSTSVGDRVSVTCKASQNVGTNVAWYQQKPGQ
SPKPLIYSATYRNSGVPDRFTGSGSGTDFTLTITNVQSKDLADYFCQQY
NRYPYTSGGGTKLEIKRAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSRVKFSRSAEPPAYQQGQNQL
YNELNLGRREEYDVLDKRRGRDPEMGGKPRRKNPQEGLYNELQKDKMAE
AYSEIGMKGERRRGKGHDGLYQGLSTATKDTYDALHMQALPPR

An exemplary nucleic acid sequence encoding the amino acid sequence of SEQ ID NO: 28 is set forth in SEQ ID NO: 29. SEQ ID NO: 29 is provided below is provided below.

[SEQ ID NO: 29]
gctctcccagtgactgccctactgcttcccctagcgcttctcctgcatg
cagaggtgaagctgcagcagtctggggctgagctggtgaggcctgggtc
ctcagtgaagatttcctgcaaggcttctggctatgcattcagtagctac
tggatgaactgggtgaagcagaggcctggacagggtcttgagtggattg
gacagatttatcctggagatggtgatactaactacaatggaaagttcaa
gggtcaagccacactgactgcagacaaatcctccagcacagcctacatg
cagctcagcggcctaacatctgaggactctgcggtctatttctgtgcaa
gaaagaccattagttcggtagtagatttctactttgactactggggcca
agggaccacggtcaccgtctcctcaggtggaggtggatcaggtggaggt
ggatctggtggaggtggatctgacattgagctcacccagtctccaaaat
tcatgtccacatcagtaggagacagggtcagcgtcacctgcaaggccag
tcagaatgtgggtactaatgtagcctggtatcaacagaaaccaggacaa
tctcctaaaccactgatttactcggcaacctaccggaacagtggagtcc
ctgatcgcttcacaggcagtggatctgggacagatttcactctcaccat
cactaacgtgcagtctaaagacttggcagactatttctgtcaacaatat
aacaggtatccgtacacgtccggaggggggaccaagctggagatcaaac
gggcggccgcaattgaagttatgtatcctcctccttacctagacaatga
gaagagcaatggaaccattatccatgtgaaagggaaacacctttgtcca
agtcccctatttcccggaccttctaagcccttttgggtgctggtggtgg
ttggtggagtcctggcttgctatagcttgctagtaacagtggcctttat
tattttctgggtgaggagtaagaggagcaggctcctgcacagtgactac
atgaacatgactccccgccgccccgggcccacccgcaagcattaccagc
cctatgccccaccacgcgacttcgcagcctatcgctccagagtgaagtt
cagcaggagcgcagagccccccgcgtaccagcagggccagaaccagctc
tataacgagctcaatctaggacgaagagaggagtacgatgttttggaca
agagacgtggccgggaccctgagatggggggaaagccgagaaggaagaa
ccctcaggaaggcctgtacaatgaactgcagaaagataagatggcggag
gcctacagtgagattgggatgaaaggcgagcgccggaggggcaaggggc
acgatggcctttaccagggtctcagtacagccaccaaggacacctacga
cgcccttcacatgcaggccctgccccctcgc

### 4. Cells

The presently disclosed subject matter provides cells comprising a dominant negative Fas polypeptide disclosed herein. In certain embodiments, the cell further comprises an antigen-recognizing receptor (e.g., a CAR or a TCR) that binds to an antigen. In certain embodiments, the dominant negative Fas polypeptide is an exogenous dominant negative Fas polypeptide. In certain embodiments, the antigen-recognizing receptor is capable of activating the cell. In certain embodiments, the dominant negative Fas polypeptide (e.g., an exogenous dominant negative Fas polypeptide) is capable of promoting an anti-tumor effect of the cell. The cells can be transduced with an antigen-recognizing receptor and an exogenous dominant negative Fas polypeptide such that the cells co-express the antigen-recognizing receptor and the exogenous dominant negative Fas polypeptide.

In certain embodiments, the cell is an immunoresponsive cell. In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, and stem cells from which lymphoid cells may be differentiated. In certain embodiments, the stem cell is a pluripotent stem cell (e.g., embryonic stem cell or induced pluripotent stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTLs or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. In certain embodiments, the cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a virus-specific T cell. In certain embodiments, the virus-specific T cell comprises an endogenous TCR that recognizes a viral antigen. In certain embodiments, the cell is a tumor-specific T cell. In certain embodiments, the tumor-specific T cell comprises an endogenous TCR that recognizes a tumor antigen.

In certain embodiments, the cell is an NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, the cell is a cell of the myeloid lineage. Non-limiting examples of cells of the myeloid lineage include monocytes, macrophages, basophils, neutrophils, eosinophils, mast cell, erythrocytes, megakaryocytes, thrombocytes, and stem cells from which myeloid cells may be differentiated. In certain embodiments, the stem cell is a pluripotent stem cell (e.g., embryonic stem cell or induced pluripotent stem cell).

The presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the presently disclosed cells have increased cell persistence. In certain embodiments, the presently disclosed cells have decreased apoptosis and/or anergy.

### 5. Compositions and Vectors

The presently disclosed subject matter provides compositions comprising a dominant negative Fas polypeptide disclosed herein (e.g., disclosed in Section 2) and an antigen-recognizing receptor disclosed herein (e.g., disclosed in Section 3). Also provided are cells (e.g., immunoresponsive cells) comprising such compositions.

In certain embodiments, the dominant negative Fas polypeptide is operably linked to a first promoter. In certain embodiments, the antigen-recognizing receptor is operably linked to a second promoter.

Furthermore, the presently disclosed subject matter provides nucleic acid compositions comprising a first polynucleotide encoding a dominant negative Fas polypeptide disclosed herein (e.g., disclosed in Section 2) and a second polynucleotide encoding an antigen-recognizing receptor disclosed herein (e.g., disclosed in Section 3). Also provided are cells comprising such nucleic acid compositions.

In certain embodiments, the nucleic acid composition further comprises a first promoter that is operably linked to the dominant negative Fas polypeptide. In certain embodiments, the nucleic acid composition further comprises a second promoter that is operably linked to the antigen-recognizing receptor.

In certain embodiments, one or both of the first and second promoters are endogenous or exogenous. In certain embodiments, the exogenous promoter is selected from the group consisting of an elongation factor (EF)-1 promoter, a CMV promoter, a SV40 promoter, a PGK promoter, a long terminal repeat (LTR) promoter and a metallothionein promoter. In certain embodiments, one or both of the first and second promoters are inducible promoters. In certain embodiments, the inducible promoter is selected from the group consisting of a NFAT transcriptional response element (TRE) promoter, a CD69 promoter, a CD25 promoter, an IL-2 promoter, an IL-12 promoter, a p40 promoter, and a Bcl-xL promoter.

The compositions and nucleic acid compositions can be administered to subjects or and/delivered into cells by art-known methods or as described herein. Genetic modification of a cell (e.g., a T cell) can be accomplished by transducing a substantially homogeneous cell composition with a recombinant DNA construct. In certain embodiments, a retroviral vector (either a gamma-retroviral vector or a lentiviral vector) is employed for the introduction of the DNA construct into the cell. For example, a first polynucleotide encoding an antigen-recognizing receptor and the second polynucleotide encoding the dominant negative Fas polypeptide can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a dominant negative Fas polypeptide and an antigen-recognizing receptor (e.g., a CAR or a TCR), a retroviral vector is generally employed for transduction, however any other suitable viral vector or non-viral delivery system can be used. The antigen-recognizing receptor and the dominant negative Fas polypeptide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-x13 IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver the dominant negative Fas polypeptide and/or the antigen-recognizing receptor disclosed herein to a cell or a subject. In certain embodiments, a CRISPR system is used to deliver the dominant negative Fas polypeptide and/or the antigen-recognizing receptor disclosed herein. In certain embodiments, zinc-finger nucleases are used to deliver the dominant negative Fas polypeptide and/or the antigen-recognizing receptor disclosed herein. In certain embodiments, a TALEN system is used to deliver the dominant negative Fas polypeptide and/or the antigen-recognizing receptor disclosed herein.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind a desired DNA sequence, and thereby guide the nuclease to cut at specific locations in genomic DNA sequences.

Polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

The resulting cells can be grown under conditions similar to those for unmodified cells, whereby the modified cells can be expanded and used for a variety of purposes.

### 6. Polypeptides and Analogs

Also included in the presently disclosed subject matter are a CD19, CD28, 4-1BB, CD8, CD3ζ, and Fas polypeptides or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, CD19, CD8, 4-1BB, CD28, CD3ζ, and Fas). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹° ° indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein (e.g., dominant negative Fas polypeptide). Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 7. Administration

The presently disclosed cells or compositions comprising thereof can be provided systemically or directly to a subject for inducing and/or enhancing an immune response to an antigen and/or treating and/or preventing a neoplasia and/or a pathogen infection. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells or NK cells in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., the thymus). Usually, at least about 1×10⁵ cells will be administered, eventually reaching about 1×10¹⁰ or more. The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed cells or their progenitors and a pharmaceutically acceptable carrier. Administration can be autologous or heterologous. For example, cells or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a presently disclosed therapeutic composition, it can be formulated in a unit dosage injectable form (solution, suspension, emulsion).

### 8. Formulations

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10⁴ and about 10¹⁰, between about 10⁵ and about 10⁹, or between about 10⁶ and about 10⁸ of the presently disclosed cells are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 9. Methods of Treatment

The presently disclosed subject matter provides methods for inducing and/or increasing an immune response in a subject in need thereof. The presently disclosed cells and compositions comprising thereof can be used for treating and/or preventing a neoplasia in a subject. The presently disclosed cells and compositions comprising thereof can be used for prolonging the survival of a subject suffering from a neoplasia. The presently disclosed cells and compositions comprising thereof can also be used for treating and/or preventing a neoplasia in a subject. The presently disclosed cells and compositions comprising thereof can also be used for reducing tumor burden in a subject. The presently disclosed cells and compositions comprising thereof can also be used for treating and/or preventing a pathogen infection or other infectious disease in a subject, such as an immunocompromised human subject. Such methods comprise administering the presently disclosed cells in an amount effective or a composition (e.g., pharmaceutical composition) comprising thereof to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

For adoptive immunotherapy using antigen-specific T cells, cell doses in the range of about 10⁶-10¹¹ (e.g., about 10) are typically infused. Upon administration of the presently disclosed cells into the host and subsequent differentiation, T cells are induced that are specifically directed against the specific antigen. The modified cells can be administered by any method known in the art including, but not limited to, intravenous, subcutaneous, intranodal, intratumoral, intrathecal, intrapleural, intraperitoneal, intra-medullary and directly to the thymus.

The presently disclosed subject matter provides methods for treating and/or preventing a neoplasm in a subject. In certain embodiments, the method comprises administering an effective amount of the presently disclosed cells or a composition comprising thereof to a subject having a neoplasia.

In certain embodiments, the neoplasia or tumors are cancers that have increased FASLG RNA expression relative to matched normal tissues of origin. See Yamamoto et al., J Clin Invest. (2019); 129(4):1551-1565, which is incorporated by reference herein.

Non-limiting examples of neoplasia include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed immunoresponsive cells and compositions comprising thereof can be used for treating and/or preventing blood cancers (e.g., leukemias, lymphomas, and myelomas) or ovarian cancer, which are not amenable to conventional therapeutic interventions.

In certain embodiments, the neoplasm is a solid cancer or a solid tumor. In certain embodiments, the solid tumor or solid cancer is selected from the group consisting of glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, lung adenocarcinoma, stomach adenocarcinoma, cervical and endocervical cancer, kidney clear cell carcinoma, testicular germ cell tumors, and aggressive B-cell lymphomas.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of a neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the immunoresponsive cells described herein in treatment prophylactically to prevent the occurrence of neoplasia until it is suitable to perform preventive surgery.

As a consequence of surface expression of an antigen-recognizing receptor that binds to a tumor antigen and a dominant negative Fas polypeptide (e.g., an exogenous dominant negative Fas polypeptide) that enhances the anti-tumor effect of the cells comprising the antigen-recognizing receptor and the dominant negative Fas polypeptide, adoptively transferred T or NK cells are endowed with augmented and selective cytolytic activity at the tumor site. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed cells or a composition comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated receptor-expressing (e.g., CAR-expressing) T cells. The incorporation of a suicide gene into the a presently disclosed antigen-recognizing receptor (e.g., CAR) gives an added level of safety with the ability to eliminate the majority of receptor-expressing (e.g., CAR-expressing) T cells within a very short time period. A presently disclosed cell (e.g., a T cell) incorporated with a suicide gene can be pre-emptively eliminated at a given timepoint post T cell infusion, or eradicated at the earliest signs of toxicity.

### 10. Kits

The presently disclosed subject matter provides kits for inducing and/or enhancing an immune response and/or treating and/or preventing a neoplasm or a pathogen infection in a subject. In certain embodiments, the kit comprises an effective amount of presently disclosed cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding a dominant negative Fas polypeptide in expressible form, which may optionally be comprised in the same or different vectors.

If desired, the cells and/or nucleic acid molecules are provided together with instructions for administering the cells or nucleic acid molecules to a subject having or at risk of developing a neoplasm or pathogen or immune disorder. The instructions generally include information about the use of the composition for the treatment and/or prevention of neoplasia or a pathogen infection. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—T Cells Engineered to Overcome Death Signaling within the Tumor Microenvironment Enhance Adoptive Cancer Immunotherapy

Introduction

Multiple variables may influence the success or failure of transferred T cells to mediate cancer regression (15). These can include the state of T cell differentiation (16) and local immune-suppressive factors present within the tumor-bearing host (17). Despite these complexities, one of the single most consistent correlates of response observed in both hematologic (2-5, 7) and solid cancers (10, 18, 19) has been the expansion and/or persistence of transferred T cells following infusion.

It was hypothesized that disruption of factors which negatively regulate T cell proliferation and survival could represent potentially actionable pathways to enhance adoptive immunotherapies. Several clinical trials tested whether cell-extrinsic approaches can improve the persistence of adoptively transferred T cells, including co-administration of an immune-checkpoint inhibitor (20, 21). However, these agents may not always efficiently enter the solid tumor microenvironment (22) and can cause non-specific immune activation resulting in systemic toxicities that do not contribute to efficacy (23). A cell-intrinsic strategy was therefore pursued to enhance function exclusively within tumor-specific T cells, thereby containing the risk of systemic toxicities and taking full advantage of the ability to reliably genetically engineer human T cells for clinical applications.

Using a pan-cancer analysis to identify candidate ligands which can limit the ability of T cells to expand and persist within the tumor-bearing host, the canonical apoptosis-inducing ligand FASLG was discovered as being preferentially expressed in the majority of human tumor-microenvironments. Further, most therapeutic T cells used for adoptive immunotherapy constitutively were found as expressing Fas, the cognate receptor for FasL. Based on these findings, a series of Fas dominant negative receptors (DNRs) were developed, which function in both primary mouse and human T cells to prevent FasL-induced apoptosis. Adoptively transferred, Fas DNR-engineered T cells showed enhanced T cell persistence and antitumor immunity without resulting in uncontrolled lymphoproliferation. Collectively, these results provide a potentially universal strategy to enhance the durability and survivability of adoptively transferred T cells in a wide range of human malignancies following ACT.

Methods and Materials

Human Specimens: Peripheral blood mononuclear cells (PBMC) were obtained from age- and sex-matched healthy donors, or melanoma patients and diffuse large B cell lymphoma (DLBCL) patients enrolled on an adoptive immunotherapy clinical protocol. All anonymous NIH Blood Bank donors and cancer patients providing PBMC samples were enrolled in clinical trials approved by the NIH Clinical Center and NCI institutional review boards. Each patient signed an informed consent form and received a patient information form prior to participation.

The Cancer Genome Atlas (TCGA) pan-cancer bioinformatics analysis: RNA-sequencing (RNA-seq) data from 26 human cancers from the TCGA dataset and matched normal tissues from the GTEx dataset was collected and analyzed by UCSC Xena in the form of normalized RNA-seq by Expectation-Maximization (RSEM) values. FASLG gene expression as normalized RSEM counts was analyzed in each. Statistics were corrected by Mann-Whitney U test. To identify genes positively correlated to FASLG expression, a pre-ranked gene set enrichment was run against all KEGG pathways in the mSigDB database. Pearson's correlation was performed on the top 1000 genes positively correlated to FASLG expression averaged across 26 TCGA histology.

Mice: Adult 6-12 week old male or female C57BL/6 NCR (B6; Ly5.2⁺) were purchased from Charles River Laboratories at NCI Frederick. B6. SJL-Ptprc^{a} Pepc^{b}/BoyJ (Ly5.1⁺), B6.129S7-Rag1Lm/Moma (Rag), B6.MRL-Fas^{j}p^{r}/J (lpr), B6.Cg-Thy1^{a}/Cy Tg(TcraTcrb)8Rest/J (pmel-1(67)), MRL/MpJ (MRL-Mp), and MRL/MpJ-Faslpr/J (MRL-lpr) mice were purchased from Jackson Laboratory. Where indicated, pmel-1 mice were crossed to Ly5.1, Rag, or Rag×lpr backgrounds. All mice were maintained under specific pathogen-free conditions. Animal experiments were approved by the Institutional Animal Care and Use Committees of the NCI and performed in accordance with NIH guidelines.

Retroviral vectors and transduction of murine and human CD8⁺ T cells: Murine and human Fas cDNA sequences were synthesized and separately cloned (Genscript) into the MSGV retroviral plasmid preceding a T2A skip sequence and selectable marker Thy1.1. Murine T cell transductions were performed as previously described(68). Briefly, Platinum-E ecotropic packaging cells (Cell BioLabs) were plated on BioCoat 10 cm dishes (Corning) overnight before transfection. The following day, 24 ug of retroviral plasmid DNA encoding MSGV-Thy1.1 (Empty), MSGV-WT-mFas-Thy1.1 (mWT), MSGV-I246N-mFas-Thy1.1 (Fas^{I246N}) or MSGV-ADD-mFas-Thy1.1 (Fas^{ΔDD}), or MSGV-1D3-28Z (anti-CD19 CAR) (71) were separately mixed with 6 ug of pCL-Eco plasmid DNA along with 60 uL of Lipofectamine 2000 (ThermoFisher) in OptiMEM the applied to the Platinum-E cells for 7h in antibiotic-free 10% medium.

Plasmids encoding human Fas mutant genes were subcloned into murine leukemia virus based SFG retroviral vector, described in Maher et al., Nat Biotechnol (2002); 20:70-75.

Medium was replaced after 7h; viral supernatant was collected from the cells after 48 hours and centrifuged to remove debris. Retroviral supernatants were spun for 2h at 2000×g 32C° on non-tissue culture treated 24-well plates that had been coated overnight in 20 ug/mL Retronectin (Takara Bio). CD8α⁺ T cells activated for 24 hours were added to plates that had all but 100 uL of viral supernatant removed, spun for 5 minutes at 1500 rpm at 32° C., then incubated overnight. The transduction was repeated a second time the next day in the manner described above. For human T cell transduction, 293T cells (69) and RD114 were used in place of Platinum-E cells and transfection and virus harvest proceeded as during the murine virus production described above.

T cell culture and Fas death assay: Human PBMC from healthy donors or patients were obtained either by leukapheresis or venipuncture and centrifuged over Ficoll-Hypaque (Lonza) gradient to remove red blood cells and isolate lymphocytes. Cells were washed twice with PBS containing 1 mM EDTA, stained with fixable cell viability dye (Thermo Fisher) in PBS, then washed twice with PBS supplemented with 2% FBS and 1 mM EDTA (FACS buffer). Untouched human CD8α⁺ T cells were isolated using a human CD8 Isolation kit (Stem Cell Technologies). Murine and human T cells and E2a-PBX leukemia cells (72) were maintained in RPMI 1640 (Gibco) with 10% heat-inactivated fetal bovine serum (FBS), 1% penicillin/streptomycin (100 U/mL and 100 ug/mL, respectively; Gibco), gentamicin (10 ug/mL), MEM non-essential amino acids (Gibco), sodium pyruvate (1 nM), GlutaMAX (2 mM), 0.011 mM 2-mercaptoethanol and amphotericin B (250 ng/mL). B16-mhgp100 tumor cells, Platinum-E cells, and 293T cells were maintained in DMEM (Gibco) supplemented with 10% FBS and the above-mentioned additives.

Untouched murine CD8α⁺ T cells were isolated from splenocytes using a MACS CD8⁺ negative selection kit (Miltenyi Biotec) and stimulated in tissue-culture treated 24-well plates with plate-bound anti-CD3 (2 ug/mL, clone 145-2C11, BD Biosciences), soluble anti-CD28 (1 ug/mL, clone 37-51, BD Biosciences) and IL-2 (5 ng/mL). Pmel-1 T cells were stimulated in whole splenocyte cultures with 1 ug/mL human gp100(25-33) peptide and IL-2 (5 ng/mL, Prometheus). Human PBMC or CD8α+ T cells were stimulated with plate-bound anti-CD3 (1 ug/mL, clone OKT3, BD Biosciences), soluble anti-CD28 (1 ug/mL, clone CD28.2, BD Biosciences) for 2 days, then given IL-2 (20 ng/mL) during the remainder of culture. Cells were stimulated for 24 hours before transduction with viral supernatant on days 1 and 2 of culture. On day 3 cells were removed from Retronectin coated plates and returned to tissue-culture treated 24-well plates or flasks. Where noted cells were grown either with vehicle or the indicated concentrations of lz-FasL, a recombinant form of oligomerized FasL(43, 52). Five to six days after stimulation, T cells were washed 2× in PBS and plated at 1-2×10⁵ cells/well in a 24-well plate with the indicated concentrations of lz-FasL and incubated at 37 C with 5% CO2 for 6 or 24 hours. Cells were then washed twice and stained for either Annexin V and PI positivity or with Live/Dead Fixable Dye (Thermo Fisher) as well as CD8a (clone 53-6.7, BD Biosciences) and Thy1.1 (clone HIS51, eBioscience).

Flow cytometry, intracellular cytokine staining and phosphoflow: Cells were stained with fixable cell viability dye (Thermo Fisher) in PBS, then washed twice with PBS supplemented with 2% FBS and 1 mM EDTA (FACS buffer). Cells were stained with the following fluorochrome-conjugated antibodies: CD3 (UCHT1), CCR7 (3D12), CD45RA (HI100), CD45RO (UCHL1), CD28 (CD28.2), CD95 (DX2) (BD Biosciences); and CD27 (M-T271), CD62L (DREG-56), CD8a (SK1), CD4 (OKT4) (BioLegend).

Murine T cells, BM, and splenocytes were stained with fixable live/dead dye followed by the following antibodies: CD3 (145-2C11), CD8a (53-6.7), V1313 (MR12-3), Ly5.1 (A20), Ly5.2 (104), CD62L (MEL-14), CD95 (Jo2), B220 (RA3-6B2) (BD Biosciences); CD44 (IM7), CD19 (6D5), CD93 (AA4.1) (BioLegend); Thy1.1 (HIS51, eBioscience). For anti-CD19 CAR detection (67) Biotin-Protein L (Genscript) was utilized.

For phosphoflow, cells were fixed and permeabilized using the BD Phosflow reagents and following the manufacturer's protocol. After permeabilization cells were stained with pAkt (S473) (D9E) and pS6 (S235/236) (D57.2.2E) from Cell Signaling. For intracellular cytokine staining, cells were stained with fixable live/dead dye in PBS, then stained for surface antibodies in FACS buffer, then fixed and permeabilized (BD Biosciences) and stained for IFNγ (XMG1.2, BD Biosciences) and IL-2 (JES6-5H4, BioLegend). For FasL staining, tumor cells were incubated with vehicle (PBS) or murine IFN-γ (100 ng ml⁻¹, Bio-Legend) for 24 hours, then stained with FasL (Kay-10) and H-2Db (KH95) (BD Biosciences). All flow cytometric data were acquired using a BD Fortessa flow cytometer (Becton Dickinson) and analyzed using FlowJo v. 9.9 software (TreeStar).

Sanger sequencing analysis: Genomic DNA from Thy1.1-enriched empty vector- or Fas^{I246N}-transduced cells was extracted using the AllPrep DNR/RNA Mini Kit (QIAGEN). Primers (IDT) were designed such that the forward primer was located in Fas upstream of the Fas^{I246N} point mutation and the reverse primer in the Thy1.1 reporter. After PCR amplification (Invitrogen) Sanger sequencing was performed.

Adoptive cell transfer, T cell enumeration, and tumor treatment: For analysis of in vivo persistence, male or female B6 mice aged 6-12 weeks received 6 Gy total body irradiation. One day later, they were injected by tail vein injection with 5×10⁵ congenically marked pmel-1 T cells transduced with a Thy1.1-containing reporter construct. Mice were sacrificed on the indicated days, and splenocytes were analyzed for homeostatic expansion of pmel-1 T cells.

For tumor treatment experiments, male or female B6 mice aged 6-12 weeks were injected with 5×10⁵ cells of a previously described B16 melanoma line (57) which overexpresses chimeric human/mouse gp100 antigen KVPRNQDWL (SEQ ID NO: 30) (a.a. 25-33) or 1×10⁶ CD19⁺E2a-PBX leukemia cells. On the indicated days, tumor-bearing mice received 6 Gy total body irradiation. Mice were left untreated as controls or received by tail vein injection indicated doses of congenically marked pmel-1 or anti-CD19 CAR-transduced T cells modified with a Thy1.1 containing reporter construct. To analyze anti-CD19 CAR-transduced T cell persistence and leukemia burden, mice were sacrificed after 14 days and cellular analysis on the spleen and BM were performed.

For experiments with MRL-Mp mice, female mice aged 8 weeks received 6 Gy total body irradiation. One day later, mice were injected with 3×10⁶ anti-CD19 CAR-transduced CD8α⁺ T cells also transduced with a Thy1.1-containing reporter construct. Age-matched MRL-lpr female mice were left unmanipulated as an ALPS positive control. All transduced T cells were bead-enriched to >92% purity using anti-Thy1.1 magnetic microbeads immediately prior to infusion (Miltenyi Biotec). All treated mice received once daily injections of 12 μg of IL-2 i.p. for 3 days. All tumor measurements were performed in a blinded fashion by an independent investigator.

T cell and tumor cell co-culture assay: After approximately 6d in culture, pmel-1 T cells were washed twice in PBS and plated in IL-2-free T cell media at 5×10⁴ cells per well in a 96-well round bottom plate. T cells were incubated either alone, with plate-bound anti-CD3/CD28 (2 μg m1⁻¹, each), with 1.5×10⁵ B16-mhgp100 cells per well for an E:T of 1:3, or with 100 ng/mL of lz-FasL. Cells were cultured together for 6 or 24 hours before being washed and stained for cell viability.

ELISA assay: Analysis of serum anti-nuclear and anti-dsDNA antibodies was performed on serum diluted 1:5; ELISA was performed according to the manufacturer's instructions (Alpha Diagnostic International).

Histopathology: Lung tissues were fixed in buffered 10% formalin and stained with H&E. Tissue sections were scored in a blinded manner by an interpreting pathologist. Scoring was as follows: 0, no specific findings; 1, mild infiltrates; 2, minimal infiltrates; 3, moderate infiltrates; 4, severe infiltrates.

Statistical Analysis: The products of perpendicular tumor diameters were plotted as the mean±SEM for each data point, and tumor treatment graphs were compared by using the Wilcoxon rank sum test and analysis of animal survival assessed using a Log-rank Mantel Cox test. For all other experiments, data were compared using either an unpaired 2-tailed Student's t test corrected for multiple comparisons by a Bonferroni adjustment or repeated measures using a 1- or 2-way ANOVA, as indicated. In all cases, P values of less than 0.05 were considered significant. Statistics were calculated using Prism 7 GraphPad software (GraphPad Software Inc.).

Results

Human Tumor Microenvironments Overexpress the Death-Inducing Ligand FASLG

Across human ACT clinical trials for both hematologic and solid cancers, in vivo T cell expansion and persistence have positively correlated with clinical responses (3-5, 10, 19). These observations led to the hypothesis that disruption of pathways that impair T cell proliferation and survival might represent potentially actionable targets for improving outcomes following adoptive transfer. To determine whether ligands that negatively modulate T cell proliferation and survival are enriched within human tumor microenvironments, RNA-sequencing data were compared using tumor-containing samples from the TCGA database relative to matched normal tissues of origin. Given recent evidence that tissues adjacent to resected tumors possess an inflamed transcriptomic profile reflective of an intermediate state between transformed and non-transformed tissues (24), expression data from the Genotype-Tissue Expression (GTEx) database (25) were used as a normal control. In total, 9,330 samples obtained from 26 different cancer types for which an appropriate matched tissue of origin was available were analyzed (Table 1). Raw data from each dataset was extracted and normalized in an identical fashion using the RNA-Seq by Expectation Maximization (RSEM) method (26).

TABLE 1

| | | TCGA |
|---|---|---|
| GTEx Tissue Type | TCGA Cancer Subtype | Abbr |
| Adrenal Gland | Adrenocortical Cancer | ACC |
| Bladder | Bladder Urothelial Carcinoma | BLCA |
| Whole Blood | Acute Lymphoblastic Leukemia | ALL |
| Brain - Amygdala | Brain Lower Grade Glioma | LGG |
| Brain - Anterior Cingulate | | |
| Cortex (Ba24) | | |
| Brain - Caudate | | |
| (Basal Ganglia) | | |
| Brain - Cerebellar | | |
| Hemisphere | | |
| Brain - Cerebellum | | |
| Brain - Cortex | | |
| Brain - Frontal Cortex (Ba9) | | |
| Brain - Hippocampus | | |
| Brain - Hypothalamus | | |
| Brain - Nucleus Accumbens | | |
| (Basal Ganglia) | | |
| Brain - Putamen | | |
| (Basal Ganglia) | | |
| Brain - Spinal Cord | | |
| (Cervical C-1) | | |
| Brain - Substantia Nigra | | |
| Brain - Amygdala | Glioblastoma Multiforme | GBM |
| Brain - Anterior Cingulate | | |
| Cortex (Ba24) | | |
| Brain - Caudate | | |
| (Basal Ganglia) | | |
| Brain - Cerebellar | | |
| Hemisphere | | |
| Brain - Cerebellum | | |
| Brain - Cortex | | |
| Brain - Frontal Cortex (Ba9) | | |
| Brain - Hippocampus | | |
| Brain - Hypothalamus | | |
| Brain - Nucleus Accumbens | | |
| (Basal Ganglia) | | |
| Brain - Putamen | | |
| (Basal Ganglia) | | |
| Brain - Spinal Cord | | |
| (Cervical C-1) | | |
| Brain - Substantia Nigra | | |
| Breast - Mammary Tissue | Breast Invasive Carcinoma | BRCA |
| Cervix - Ectocervix | Cervical & Endocervical Cancer | CESC |
| Cervix - Endocervix | | |
| Kidney - Cortex | Kidney Clear Cell Carcinoma | KIRC |
| Kidney - Cortex | Kidney Renal Papillary Cell | KIRP |
| | Carcinoma | |
| Colon - Sigmoid | | |
| Colon - Transverse | Colon Adenocarcinoma | COAD |
| Colon - Sigmoid | Rectum Adenocarcinoma | READ |
| Esophagus - | Esophageal Carcinoma | ESCA |
| Gastroesophageal Junction | | |
| Esophagus - Mucosa | | |
| Liver | Liver Hepatocellular Carcinoma | LIHC |
| Lung | Lung Adenocarcinoma | LUAD |
| Lung | Lung Squamous Cell Carcinoma | LUSC |
| Spleen | Diffuse Large B-Cell Lymphoma | DLBC |
| Adipose - Subcutaneous | Sarcoma | SARC |
| Artery - Tibial | | |
| Nerve - Tibial | | |
| Muscle - Skeletal | | |
| Fallopian Tube | Ovarian Serous | OV |
| Ovary | Cystadenocarcinoma | |
| Prostate | Prostate Adenocarcinoma | PRAD |
| Pancreas | Pancreatic Adenocarcinoma | PAAD |
| Skin - Not Sun Exposed | Skin Cutaneous Melanoma | SKCM |
| (Suprapubic) | | |
| Skin - Sun Exposed | | |
| (Lower Leg) | | |
| Stomach | Stomach Adenocarcinoma | STAD |
| Testis | Testicular Germ Cell Tumor | TGCT |
| Thyroid | Thyroid Carcinoma | THCA |
| Uterus | Uterine Carcinosarcoma | UCS |
| Uterus | Uterine Corpus Endometrioid | UCEC |
| | Carcinoma | |

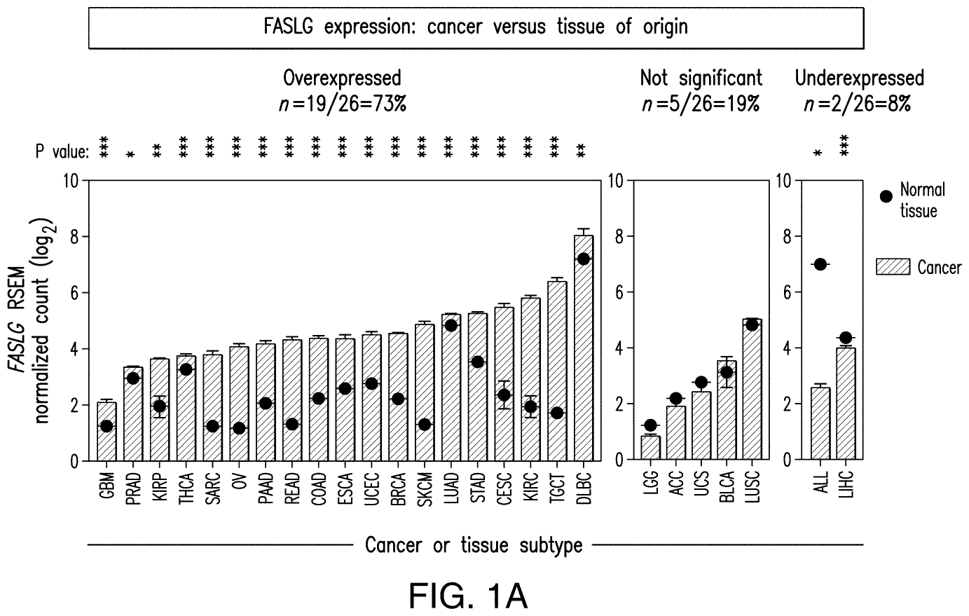

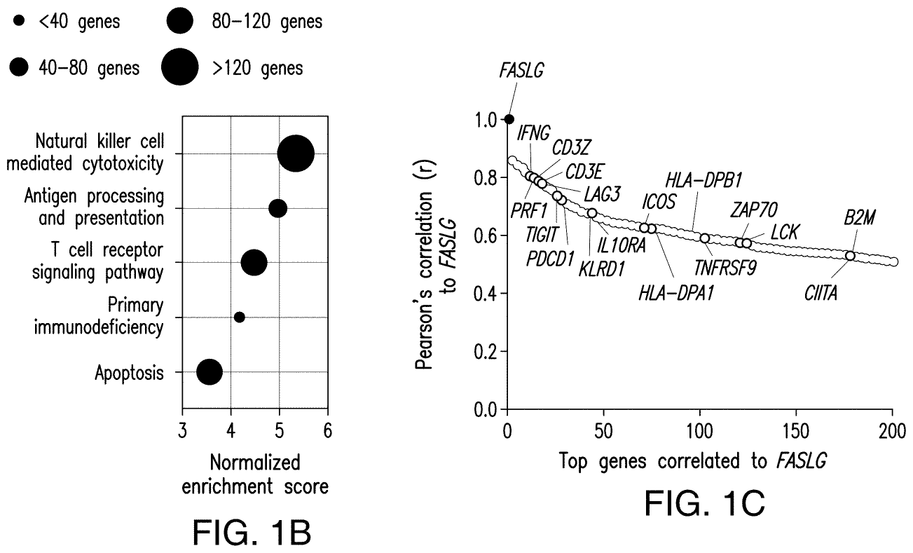

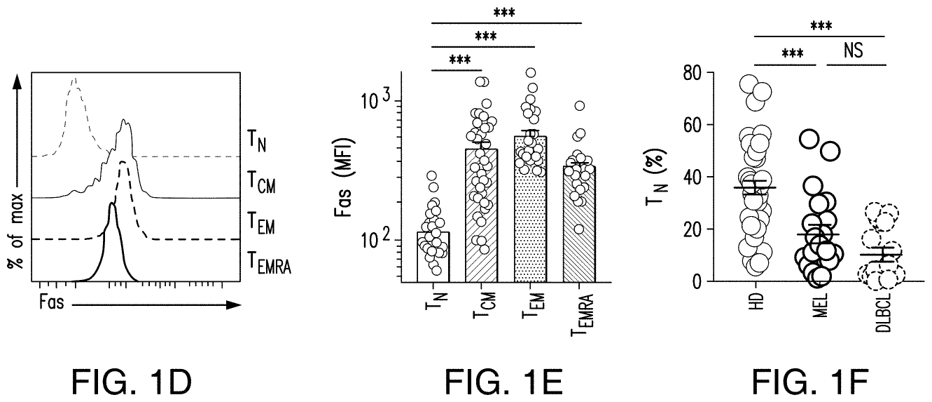

It was discovered that expression of FASLG, the gene encoding the canonical inducer of cellular apoptosis FasL (CD178), was overexpressed in the majority of evaluated cancer types relative to normal tissues (FIG. 1A). This included both immunotherapy responsive cancers, such as cutaneous melanoma (SKCM), renal clear cell carcinoma (KIRC), lung adenocarcinoma (LUAD), and gastro-esophageal carcinomas (STAD/ESCA), as well as cancers relatively recalcitrant to current immunotherapies, such as breast cancer (BRCA), colorectal adenocarcinoma (READ/COAD), glioblastoma multiforme (GMB), ovarian cancer (OV), pancreatic adenocarcinoma (PAAD), and prostate adenocarcinoma (PRAD). In total, 73% (19/26) of the human tumor types evaluated exhibited significant differential expression of FASLG within the tumor mass relative to a normal tissue control (P<0.05 to P<0.001; Mann-Whitney U test, Bonferroni-corrected). By contrast, only 19% (5/26) of cancer types did not exhibit significant differential expression and only a minority (8%; 2/26) showed evidence of reduced FASLG-expression in tumor samples vs. normal tissue.

To gain greater insight into the nature of FASLG expression within human tumor microenvironments, gene-set enrichment analysis (GSEA) (27) using genes positively correlated with FASLG across all 26 evaluated cancer types was performed (FIG. 1B). Expression profiles for many immune-related pathways, including NK cell cytotoxicity, antigen processing and presentation, TCR signaling, primary immune deficiency, and apoptosis, were each significantly enriched (nominal P-value<0.001, FDR q value<0.001). Consistent with these findings, examination of the top 200 genes positively correlated with FASLG revealed a predominance of markers associated both with lymphocyte activation, such as IFNG, PRF 1, 41BB, and LCOS, and immune counter-regulation, such as PDCD1, LAGS, and IL10RA (FIG. 1C and Table 2). Taken together, these data indicated that a death-inducing ligand which might compromise T cell survival is significantly overexpressed in the majority of human cancer microenvironments and is highly correlated to expression signatures of immune activation and regulation.

TABLE 2

| Gene | r | Gene | r | Gene | r | Gene | r | Gene | r |
|---|---|---|---|---|---|---|---|---|---|
| SLA2 | 0.8580 | CCL5 | 0.6757 | NCKAP1L | 0.6194 | ZAP70 | 0.5731 | PTPRCAP | 0.5407 |
| CD8A | 0.8539 | ARHGAP9 | 0.6747 | LILRB1 | 0.6157 | NCR1 | 0.5723 | CD96 | 0.5387 |
| CCR5 | 0.8510 | KLRD1 | 0.6742 | SIT1 | 0.6144 | MS4A6A | 0.5721 | STAT1 | 0.5375 |
| CD2 | 0.8440 | SLFN12L | 0.6739 | GNGT2 | 0.6142 | LCK | 0.5720 | FCER1G | 0.5374 |
| NKG7 | 0.8394 | ARHGAP30 | 0.6597 | C1QA | 0.6137 | ARHGAP15 | 0.5680 | CST7 | 0.5370 |
| GZMA | 0.8383 | ZNF683 | 0.6593 | TNFAIP8L2 | 0.6107 | CD86 | 0.5667 | IGFLR1 | 0.5366 |
| KLRK1 | 0.8337 | IL10RA | 0.6583 | APOL3 | 0.6092 | LAIR1 | 0.5656 | TRAF31P3 | 0.5364 |
| CRTAM | 0.8205 | IL18BP | 0.6576 | FCGR1B | 0.6073 | GBP1 | 0.5605 | HLA-DMA | 0.5362 |
| CXCR6 | 0.8097 | TRAT1 | 0.6512 | TLR8 | 0.6069 | CD200R1 | 0.5605 | CYBB | 0.5360 |
| SIRPG | 0.8039 | ABCD2 | 0.6506 | DOCK2 | 0.6052 | CD4 | 0.5594 | LAT | 0.5342 |
| IFNG | 0.8036 | GPR65 | 0.6502 | IKZF1 | 0.6031 | GBP2 | 0.5590 | TNFRSF1B | 0.5342 |
| UBASH3A | 0.8005 | SASH3 | 0.6493 | LTA | 0.6025 | ABI3 | 0.5580 | ITGB2 | 0.5334 |
| EOMES | 0.8000 | CD6 | 0.6478 | ARHGAP25 | 0.6021 | HLA-E | 0.5577 | CD3G | 0.5329 |
| PRF1 | 0.7911 | SLAMF7 | 0.6440 | HLA-DPB1 | 0.6015 | PLEK | 0.5570 | TBC1D10C | 0.5304 |
| CD247 | 0.7882 | CYTH4 | 0.6436 | TTC24 | 0.6014 | LAPTM5 | 0.5557 | TRIM22 | 0.5290 |
| PYHIN1 | 0.7822 | FAM78A | 0.6416 | C1QC | 0.5971 | SAMHD1 | 0.5553 | JAK3 | 0.5289 |
| CD3E | 0.7818 | PTPRC | 0.6414 | GIM4P2 | 0.5962 | ZNF80 | 0.5547 | CIITA | 0.5288 |
| CD3D | 0.7780 | XCL2 | 0.6397 | BTN3A2 | 0.5936 | CORO1A | 0.5542 | B2M | 0.5280 |
| LAG3 | 0.7708 | RASAL3 | 0.6395 | WIPF1 | 0.5896 | IL16 | 0.5531 | GAB 3 | 0.5265 |
| GZMH | 0.7693 | CD74 | 0.6386 | TIFAB | 0.5893 | CLEC2D | 0.5506 | SIGLEC10 | 0.5264 |
| CCL4 | 0.7582 | IRF1 | 0.6385 | GIM4P4 | 0.5892 | C5orf56 | 0.5503 | VAV1 | 0.5263 |
| CXCR3 | 0.7568 | SEPT1 | 0.6366 | TNFRSF9 | 0.5889 | GBP5 | 0.5494 | TAP1 | 0.5254 |
| GPR174 | 0.7559 | ITK | 0.6359 | APOBEC3H | 0.5887 | LILRB2 | 0.5467 | CXorf21 | 0.5239 |
| GZMK | 0.7358 | CD53 | 0.6358 | FCGR3A | 0.5885 | CARD16 | 0.5464 | CD160 | 0.5232 |
| TIGIT | 0.7333 | BIN2 | 0.6340 | IL18RAP | 0.5873 | HLA-DQA1 | 0.5463 | NCF1 | 0.5203 |
| ITGAL | 0.7237 | GRAP2 | 0.6332 | CCR2 | 0.5869 | P2RY13 | 0.5462 | GIMAP5 | 0.5200 |
| IL12RB1 | 0.7188 | AC008964.1 | 0.6305 | CD48 | 0.5851 | HLA-DOA | 0.5458 | PTPN7 | 0.5199 |
| LCP2 | 0.7133 | MYO1F | 0.6296 | CD72 | 0.5851 | FMNL1 | 0.5452 | FERMT3 | 0.5190 |
| PDCD1 | 0.7126 | IL21R | 0.6285 | LAP3 | 0.5846 | SCIMP | 0.5446 | LST1 | 0.5189 |
| SAMD3 | 0.7113 | BTN3A3 | 0.6265 | APOBEC3D | 0.5836 | C1orf162 | 0.5446 | ITGAE | 0.5188 |
| FAM26F | 0.7050 | ICOS | 0.6242 | SELPLG | 0.5808 | IGSF6 | 0.5438 | IL2RB | 0.5180 |
| SNX20 | 0.6990 | PVRIG | 0.6240 | DOK2 | 0.5803 | PSMB9 | 0.5435 | SAMSN1 | 0.5160 |
| CTSW | 0.6979 | SLAMF8 | 0.6235 | AD000671.6 | 0.5802 | EV12A | 0.5434 | BTN2A2 | 0.5152 |
| FCRL6 | 0.6945 | C1QB | 0.6235 | AIF1 | 0.5797 | HLA-DRB1 | 0.5421 | GMFG | 0.5139 |
| PSTPIP1 | 0.6945 | HLA-DPA1 | 0.6229 | SLA | 0.5780 | FYB | 0.5420 | GIMAP7 | 0.5119 |
| HCST | 0.6833 | HLA-DRA | 0.6224 | APOBEC3G | 0.5778 | PARVG | 0.5419 | APOL6 | 0.5112 |
| SLAMF6 | 0.6826 | TBX21 | 0.6219 | GBP4 | 0.5776 | P2RY10 | 0.5414 | NLRC5 | 0.5099 |
| SPN | 0.6803 | BTN3A1 | 0.6217 | ACAP1 | 0.5762 | LILRB4 | 0.5412 | LY9 | 0.5092 |
| CXCL9 | 0.6795 | FCGR1A | 0.6216 | SP140 | 0.5752 | WAS | 0.5410 | GPR31 | 0.5087 |
| KLRC4- | 0.6773 | KLRC4 | 0.6200 | EVI2B | 0.5744 | C15orf 53 | 0.5408 | AKNA | 0.5087 |
| KLRK1 | | | | | | | | | |

Next, whether Fas (CD95), the cognate receptor for FasL, is expressed on the surface of T cells used for clinical adoptive immunotherapy was determined. Fas was previously found as being expressed on all non-naïve human T cell subsets from healthy donors (HD), including central memory (TCM), effector memory (TEM), and effector memory T cells co-expressing CD45RA (TEMRA) (28, 29). The frequency of CD8α⁺ T cell subsets and each subset's Fas expression in patients with melanoma and aggressive B cell lymphomas from apheresis products used to generate therapeutic T cells for ACT was analyzed. In these patients. It was found that there was high expression of Fas on the TCM, TEM, and TEMRA subsets (FIGS. 1D and 1E). Additionally, the frequency of naïve CD8α⁺ T cells (TN) in these patients relative to a group of age-matched HDs was compared. It was found that HDs had a significantly higher percentage of Fas⁻ TN cells compared to melanoma and lymphoma patients (FIG. 1F), a finding likely reflecting the influence of prior immune-stimulating and lymphodepleting therapies in the cancer patients analyzed (5, 30, 31). Thus, a significant proportion of human T cells used for ACT expressed a known death receptor and these cells were transferred into tumor microenvironments enriched in expression of its cognate ligand.

T Cells Engineered with Fas Dominant Negative Receptors Prevent FasL-Mediated Apoptosis

The findings indicated that patient-derived T cells used for adoptive immunotherapy were skewed towards Fas-expressing subsets, which were subsequently transferred into FASLG-enriched tumor microenvironments. Based on these data, whether disruption of Fas signaling within adoptively transferred T cells might prevent their apoptosis and improve in vivo persistence was next investigated. In addition to triggering T cell apoptosis, FasL is also an essential effector molecule for T cell-mediated tumor killing (32). Further, systemic administration of either an anti-FasL antibody or Fas-Fc fusion protein can induce toxicities, including development of a lymphoproliferative syndrome and accumulation of an abnormal population of double-negative (DN) CD3⁺B220⁺CD4⁻CD8⁻TCRα/β⁺ lymphocytes (33, 34). For these reasons, a cell-intrinsic genetic engineering strategy was pursued to disable Fas signaling only within tumor-reactive T cells to maintain antitumor potency and minimize the risk of systemic toxicity.

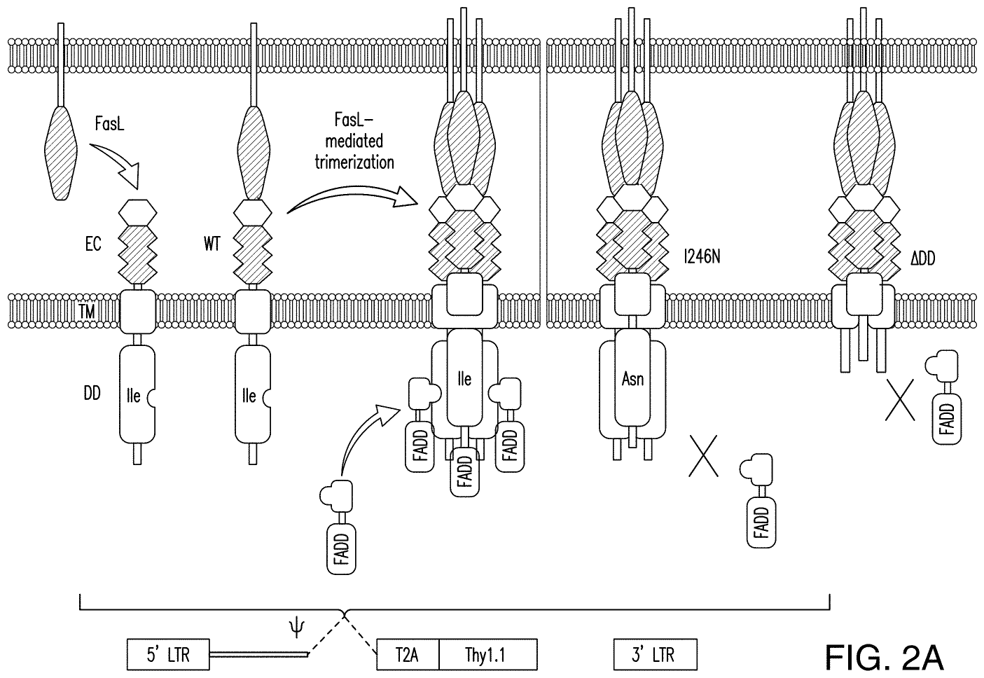

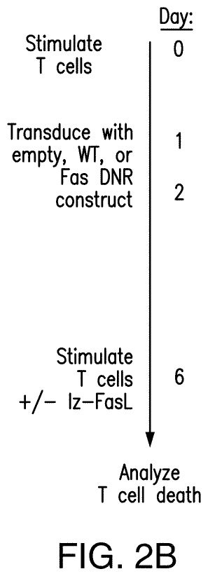

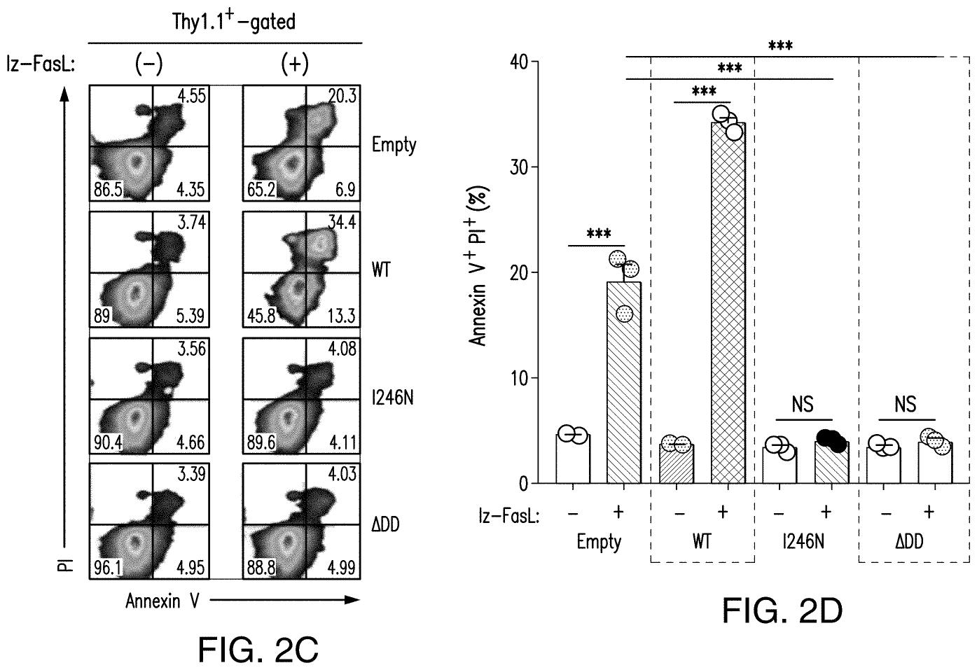

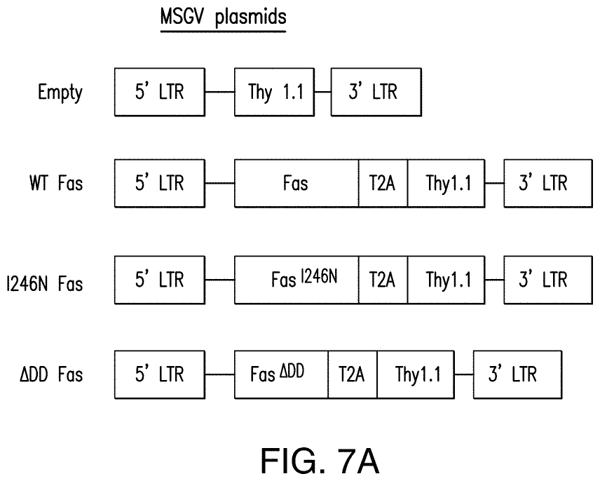

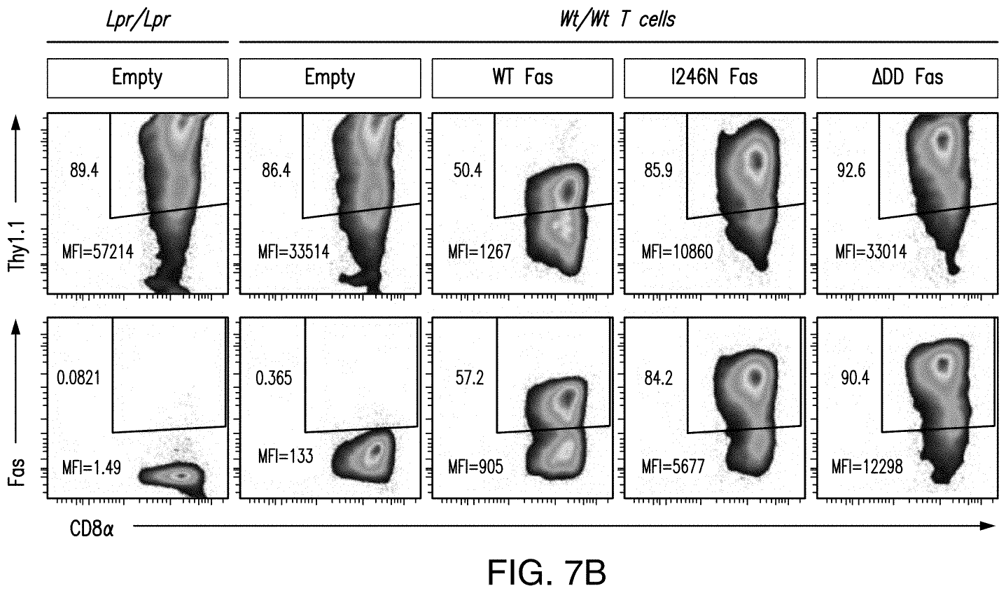

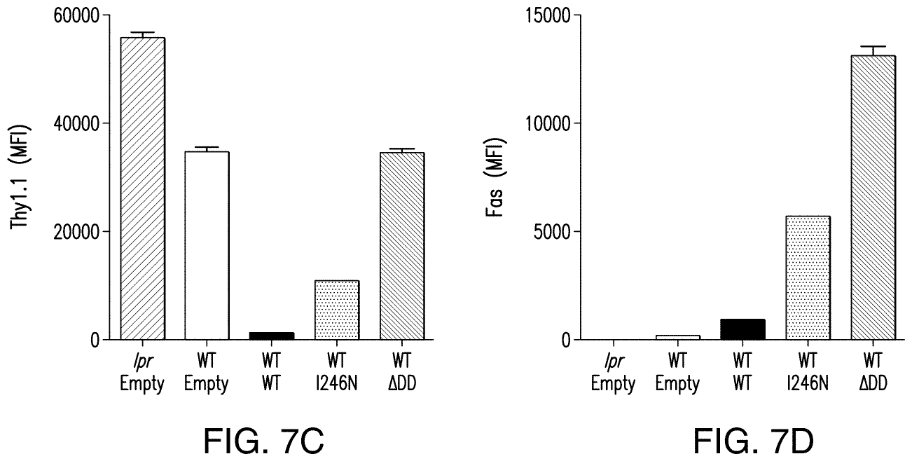

Physiologically, FasL initiates apoptotic signaling by first inducing oligomerization of Fas receptors into trimers or larger oligomers at the cell membrane (FIG. 2A) (35). Fas oligomers recruit the intracellular adapter molecule Fas-associated via death domain (FADD) through homotypic death domains (DD) present in each molecule (36, 37). Aggregation of FADD recruits the cysteine-aspartic acid protease pro-Caspase 8 (38) through homologous death effector domains in each molecule, forming the death inducing signaling complex (DISC) that can initiate the apoptotic signaling cascade (39). Based on this mechanism of action, it was hypothesized that overexpression of mutated Fas variants genetically altered to prevent FADD binding would function as a dominant negative receptor (DNR) when expressed in Fas-competent wild type (WT) T cells used for adoptive immunotherapy. Presently, virus-based constructs are the most commonly used methods to stably modify human T cells for clinical application (40). Therefore, a series of retroviral constructs were created encoding the murine Fas sequence in which either an asparagine residue was substituted for an isoleucine at position 246 of the DD (Fas^{I246N}), a naturally occurring mutant of murine Fas which is unable to bind FADD (41, 42), or a Fas mutant in which the majority of the intracellular DD was truncated (del aa222-306; Fas^{ΔDD}) to prevent FADD binding (FIGS. 2A and 7A). As controls, both an empty vector construct as well as a construct encoding the complete WT sequence of Fas (Fas^{WT}) were generated. To identify transduced cells, all vectors contained a Thy1.1 reporter separated from Fas using a T2A “self-cleavage” sequence.

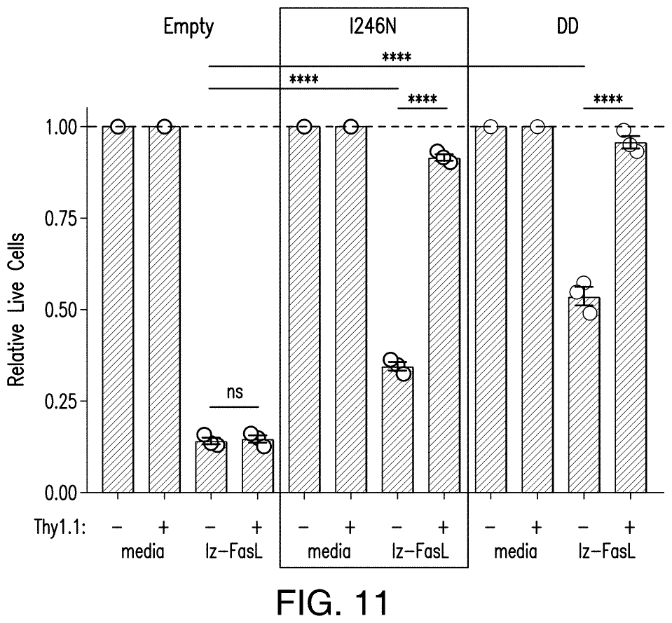

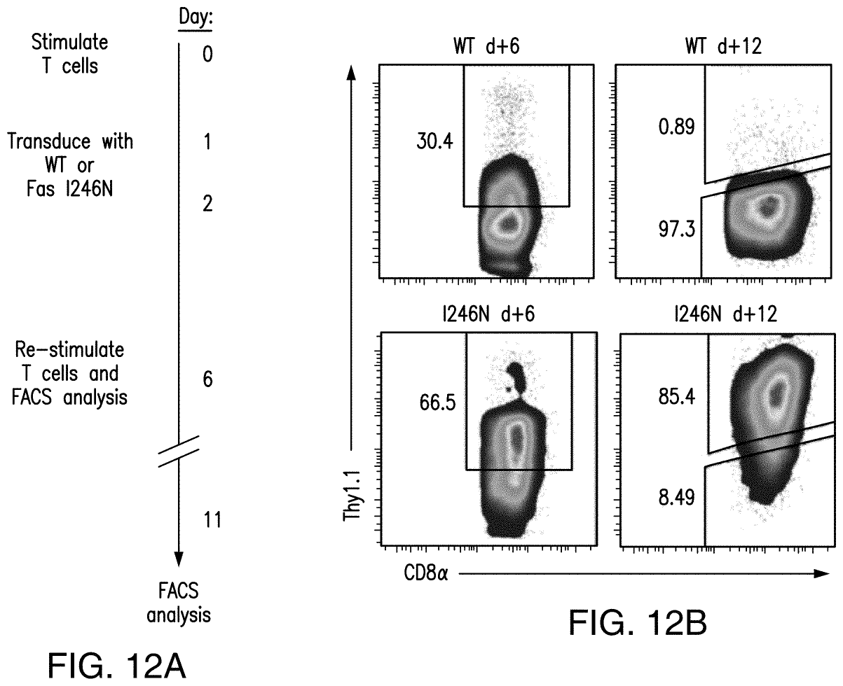

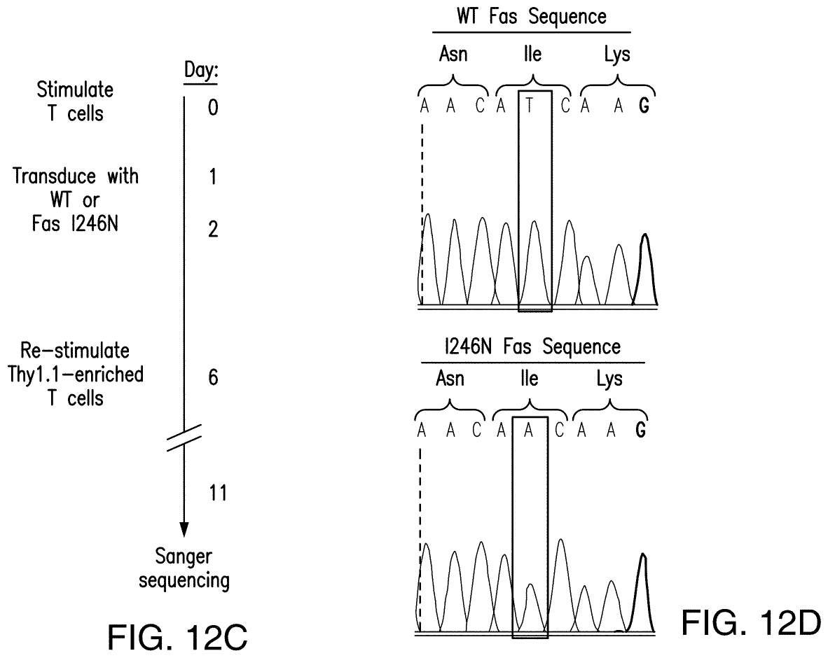

T cells were isolated from Fas-competent WT mice, activated in the presence of IL-2, and transduced with the empty, Fas^{WT}, Fas^{I246N}, or Fas^{ΔDD} constructs (FIG. 2B). Phenotypic analysis 6d following activation and transduction revealed high transduction efficiencies for all constructs as measured by Thy1.1 expression (FIGS. 7B and 7C). Notably, ectopic Fas expression was measurably higher than endogenous levels of Fas expression for constructs containing either the WT (6.8-fold higher Fas MFI) or mutant Fas variants (43-fold and 98-fold higher Fas MFI for Fas^{I246N} and Fas^{ΔDD}, respectively) (FIGS. 7B and 7D). After 6 days in culture, transduced T cells were stimulated with recombinant FasL molecules oligomerized through a leucine zipper domain (1z-FasL) to mimic the function of membrane-bound FasL (43), or left untreated as controls. In the absence of lz-FasL, T cells transduced with each of the constructs remained similarly viable (FIG. 2C). However, following exposure to lz-FasL, a significant proportion of Thy1.1⁺ T cells transduced either with the empty vector control or Fas^{WT} converted to an apoptotic Annexin V⁺PI⁺ population (FIGS. 2C and 2D; P<0.001). Interestingly, overexpression of Fas^{WT} consistently resulted in higher levels of apoptosis relative to empty vector-transduced T cells, indicating that expression of Fas above physiologic levels sensitized T cells to FasL-mediated cell death. By contrast, T cells transduced either with the Fas^{I246N} or Fas^{ΔDD} vectors were almost completely protected from lz-FasL-induced apoptosis. Among pools of T cells transduced with Fas^{I246N} or Fas^{ΔDD}, protection from apoptosis was confined to the Thy1.1⁺ populations, indicating a cell-intrinsic function of the Fas DNRs (FIG. 11). This showed that Fas^{I246N} and Fas^{ΔDD} may also protect neighboring T cells from apoptosis, likely by functioning as a “sink” for local FasL. In T cells modified with Fas^{I246N}, neither functional nor genetic evidence of reversion to the WT sequence was found. Selective enrichment for T cells modified with Fas^{I246N} compared with Fas^{WT} following serial in vitro restimulations was measured, indicating that the DNR remained functionally intact over time (FIGS. 12A and 12B). Further, Sanger sequencing of serially restimulated, Fas^{I246N}-transduced T cells showed no evidence of reversion of the I246N point mutation to the WT Fas sequence (FIGS. 12C and 12D). Thus, overexpression of Fas variants disabled their ability to bind FADD function in a dominant negative manner to prevent FasL-mediated apoptosis in WT T cells.

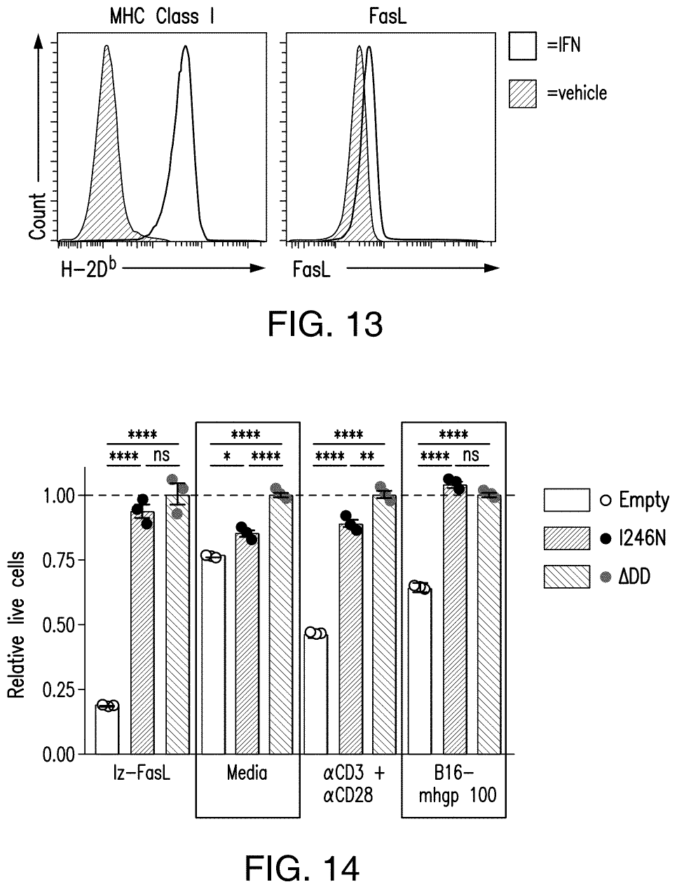

Finally, it was sought to ascertain whether the Fas DNRs afforded protection from other apoptosis-inducing stimuli that adoptively transferred T cells might encounter in vivo. These include activation-induced cell death (AICD), cytokine withdrawal, and proximity to tumor cells. For these assays, pmel-1 T cells specific for the cancer antigen gp100 and B16 melanoma engineered to express human gp100 (B16 cells) were utilized. Although B16 cells did not express FasL at rest, FasL expression was measurably upregulated following incubation with IFN-γ (FIG. 13). pmel-1 T cells transduced with Fas^{I246N} or Fas^{ΔDD} were equally protected from apoptosis triggered by either lz-FasL or tumor coculture (FIG. 14). By contrast, transduction of T cells with Fas^{ΔDD} resulted in significantly greater cell viability following AICD induction through anti-CD3/CD28 restimulation or acute cytokine withdrawal relative to cells modified with Fas^{I246N}. These findings were potentially attributable to the ability of the Fas^{I246N} variant to bind to FADD with reduced efficiency under certain conditions (73). Therefore, the present disclosure subsequently focused exclusively on the Fas^{ΔDD} DNR for all in vivo experiments given its superior functional attributes. This permitted to more clearly determine the influence of removing Fas signaling on the in vivo function of adoptively transferred T cells.

Adoptive Transfer of T Cells Engineered with Fas DNR Results in Superior Persistence

Whether expression of a Fas DNR in T cells would result in superior in vivo persistence following adoptive transfer into a tumor-bearing host was determined next.

Congenically marked, gene-modified pmel-1 T cells were adoptively transferred into sublethally irradiated Thy1.1⁻C57BL/6 (B6) mice to induce homeostatic proliferation, and the expansion and persistence of transferred cells over time was measured. T cells transduced with Fas^{ΔDD} or empty vector control were identified by expression of the Thy1.1 reporter gene. To measure T cell proliferation, T cells were co-stained for the cellular proliferation marker Ki-67.

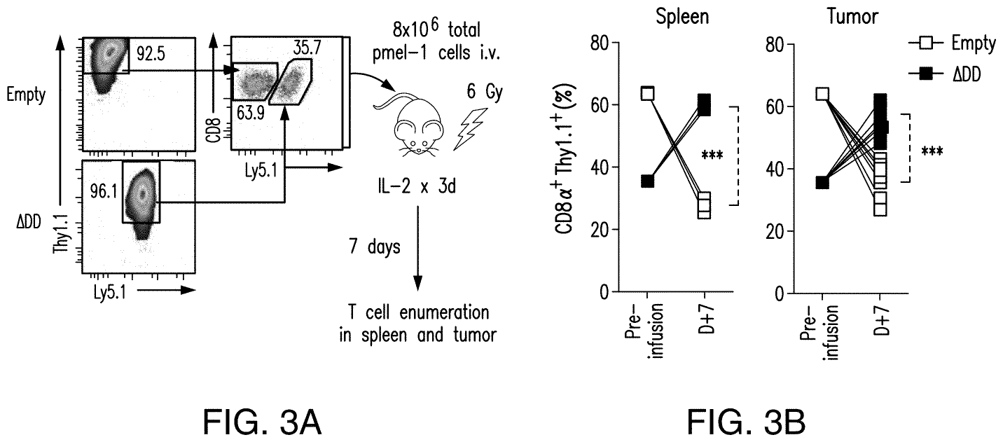

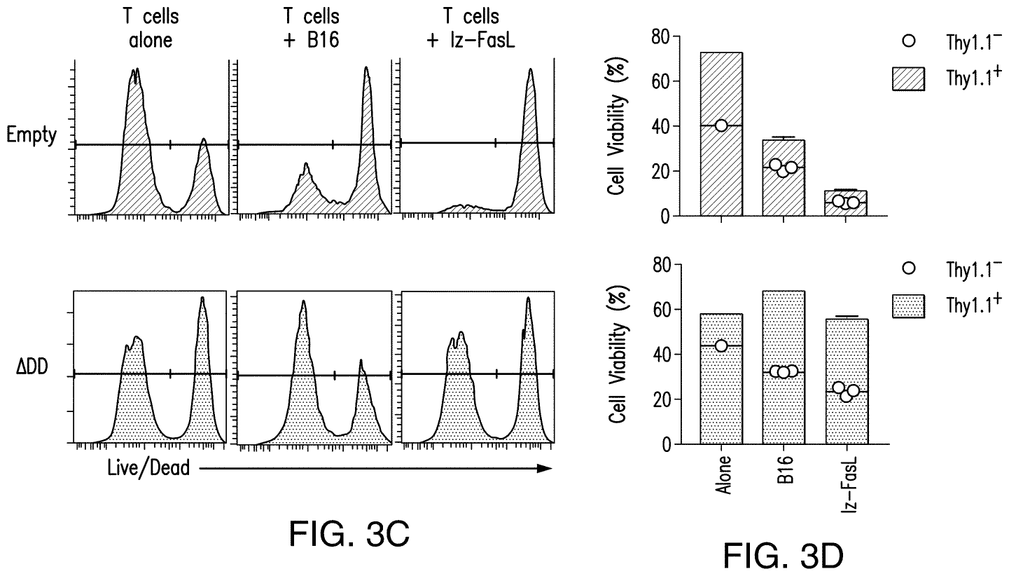

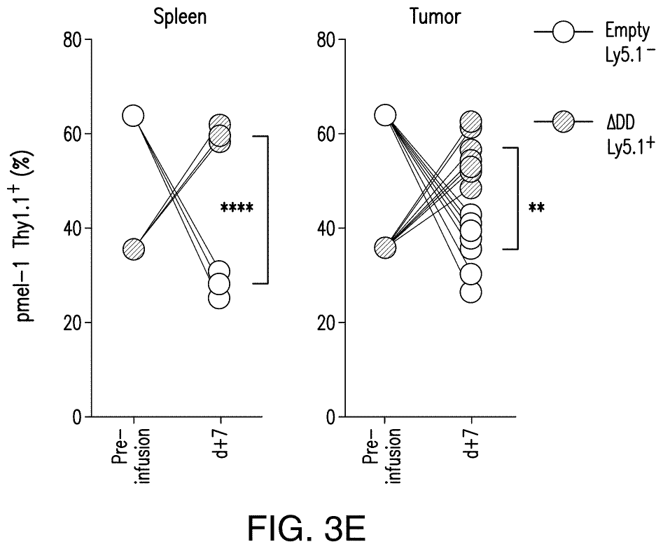

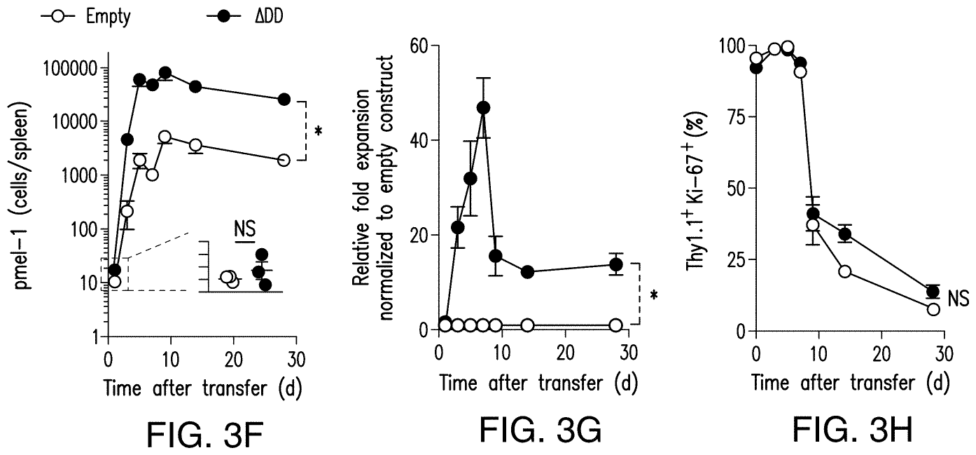

One day after transfer, Fas^{ΔDD}- and empty vector-modified pmel-1 T cells engrafted at similar levels and almost uniformly expressed Ki-67 (FIGS. 3F-3H). Beginning within 3 days of transfer, a multi-log expansion of both populations of modified cells was measured. However, at the peak of expansion, an approximately 50-fold greater increase in the numbers of Fas^{ΔDD}-modified T cells relative to control-modified cells was observed. This in turn led to a more than 10-fold-higher level of persistence of Fas DNR-modified T cells on day 30 (FIGS. 3F and 3G). Over time, a comparable reduction in Ki-67 expression on both engineered T cell populations (FIG. 3H) was observed, which correlated with reconstitution of the host's endogenous T cell compartment. These data suggested that the in vivo proliferation was comparable between the two engineered T cell populations. However, Fas DNR-modified T cells demonstrated superior overall expansion and intermediate-term persistence, likely through a reduction in apoptosis.

Next, it was sought to ascertain whether genetic modification with the Fas DNR resulted in superior T cell persistence within the TME. To ensure that modified T cells were exposed to the same microenvironmental factors within any given tumor, a coinfusion experiment was performed.

Congenically distinguishable pmel-1 CD8D⁺ T cells specific for the cancer antigen gp100 were obtained from either a Ly5.1⁻/Thy1.1⁻ or Ly5.1⁺/Thy1.1⁻ background. Cells were transduced with the Fas^{ΔDD} DNR or a Thy1.1-expressing empty vector control, respectively. Thy1.1-expressing, transduced T cells were subsequently purified using anti-Thy1.1 microbeads, recombined in a roughly 1:1 ratio, and then co-infused into sublethally irradiated Ly5.1⁻/Thy1.1⁻ mice bearing 10d established B16 melanoma tumors (FIG. 3A). As is currently done in many ACT clinical trials for solid tumors, treated mice received a limited course of IL-2 following transfer (13, 18, 44-46). Seven days following infusion, both spleens and tumors of recipient mice were harvested and analyzed for the presence of adoptively transferred, genetically modified, Thy1.1⁺pmel-1 T cells. Significant enrichment of Ly5.1_Thy1.1⁺Fas^{ΔDD}-modified T cells relative to Ly5.1_Thy1.1⁺empty vector-modified T cells in both the spleen and tumor of recipient mice was consistently found (FIGS. 3B and 3E; P<0.01, P<0.001). To test whether T cells engineered with the Fas^{ΔDD} DNR could enhance T cell survival in a microenvironment enriched in tumor cells, an in vitro co-culture assay was performed. Pmel-1 T cells expressing either the Fas^{ΔDD} or an empty vector control were plated alone in the absence of IL-2 overnight or co-cultured with B16 melanoma tumors. As a positive control for cell death, T cells were cultured in the presence of lz-FasL. In this experiment, T cells were not bead-enriched for Thy1.1 to enable an additional internal control. After 24h, T cell viability was accessed by FACS analysis. While substantial cell death was induced in empty vector-transduced pmel-1 T cells by either co-culturing with B16 or addition of lz-FasL, this was not observed in Fas^{ΔDD}-transduced counterparts (FIG. 3C). Moreover, non-transduced cells in both groups showed comparable cell viability in response to B16 co-culture or lz-FasL (FIG. 3D). Together, these results indicated that genetic engineering with a Fas DNR enhanced engraftment and survivability of tumor-reactive T cells following adoptive cell transfer and exposure to a tumor-enriched microenvironment.

ACT of Fas DNR-Modified T Cells does not Result in an ALPS Phenotype

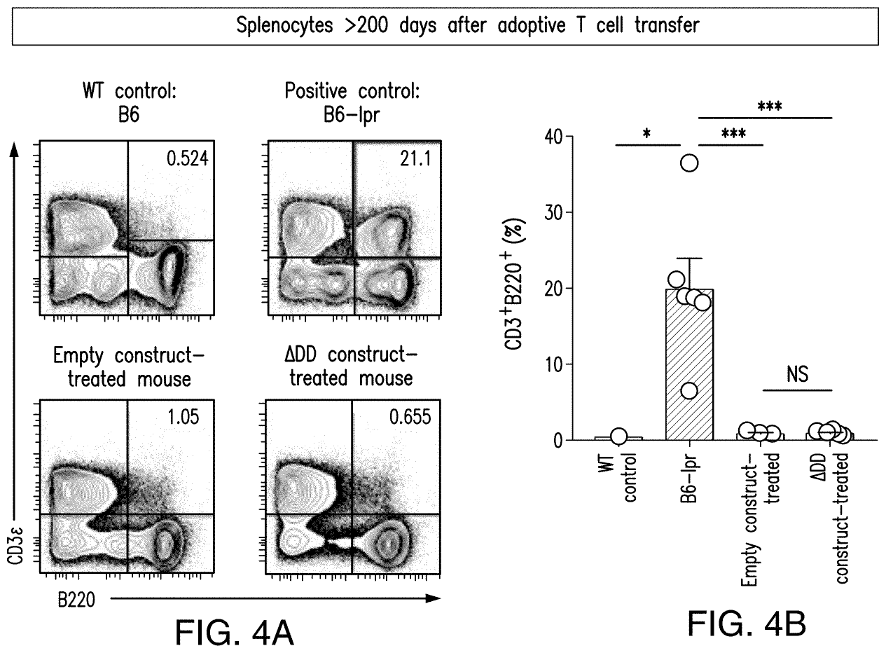

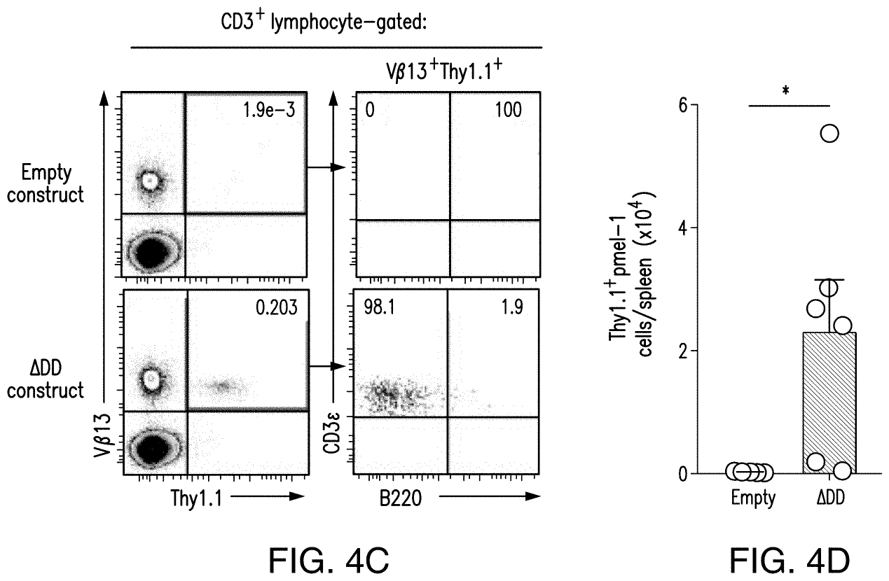

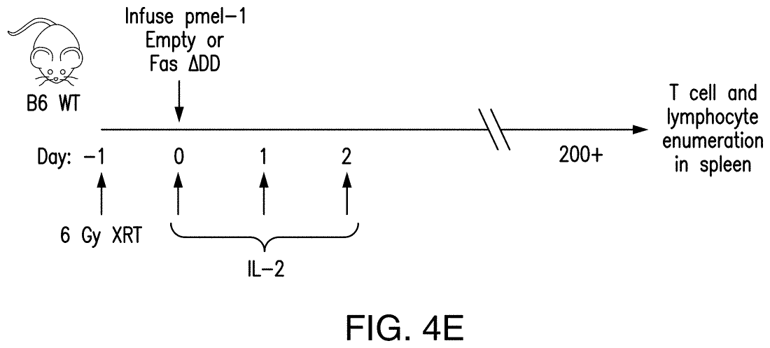

Mice and humans with germline defects in components of normal apoptotic signaling, such as Fas, can develop profound alterations in normal lymphocyte homeostasis and development. These abnormalities, collectively referred to as autoimmune lymphoproliferative syndrome (ALPS), include the accumulation of an aberrant CD3⁺B220⁺CD4⁻CD8⁻ lymphocyte population and development of auto-antibodies resulting in impaired survival (47, 48). Given the potential safety concerns related to disabling normal Fas signaling in mature T cells, detailed, long-term, immune-monitoring of animals that received Fas^{ΔDD} DNR-modified T cells more than 6 months prior was performed (FIG. 4E). This time point was chosen as mice with germline defects in Fas typically develop overt clinical manifestations within the first 3.5-5 months of life, depending on the background strain (49, 50). Using unmanipulated WT and Fas-deficient lpr/lpr mice as respective negative and positive controls for the ALPS phenotype, the frequency of CD3⁺B220⁺lymphocytes in the spleens of mice who had previously received ACT of Vβ3 13⁺ pmel-1 T cells modified with the Fas^{ΔDD} DNR or an empty vector control was assessed. As expected, the spleens of lpr/lpr mice exhibited a significant accumulation of abnormal CD3^{+B}220⁺lymphocytes relative to WT controls (FIGS. 4A and 4B; P<0.05, P<0.001). By contrast, neither mice receiving T cells modified with the empty vector control or Fas DNR exhibited a significant increase in this population. To exclude the transformation of our modified T cell population, we assessed the long-term persistence and phenotype of the transferred Vβ3 13⁺ Thy 1.1⁺ engineered T cells. At more than 200 days, T cells engineered with Fas^{ΔDD} DNR persisted at higher numbers than to cells modified with the empty vector control (FIGS. 4C and 4D; P<0.05). Long-term-persisting Fas DNR-modified T cells maintained a conventional CD3^{+B}220⁻phenotype. These data showed that adoptively transferred pmel-1 T cells expressing the Fas DNR did not undergo abnormal lymphoproliferation in B6 hosts.

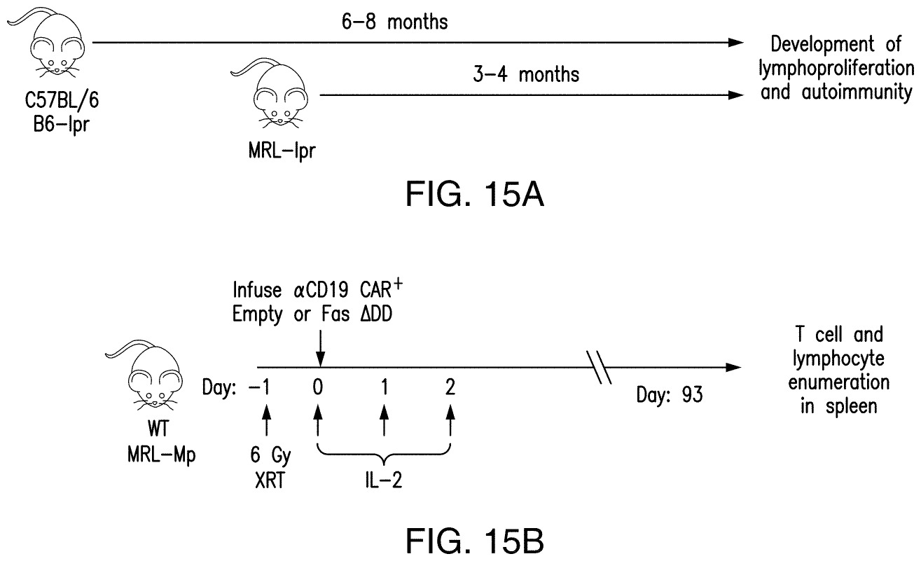

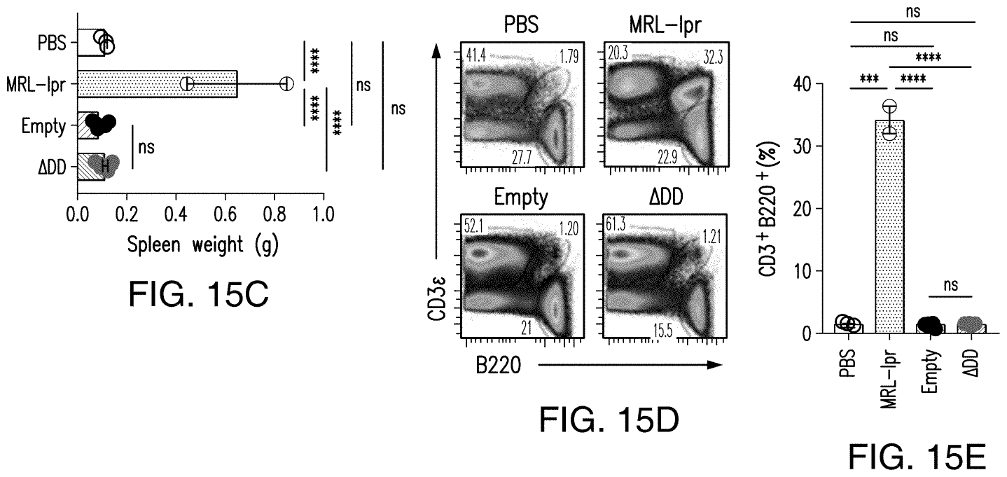

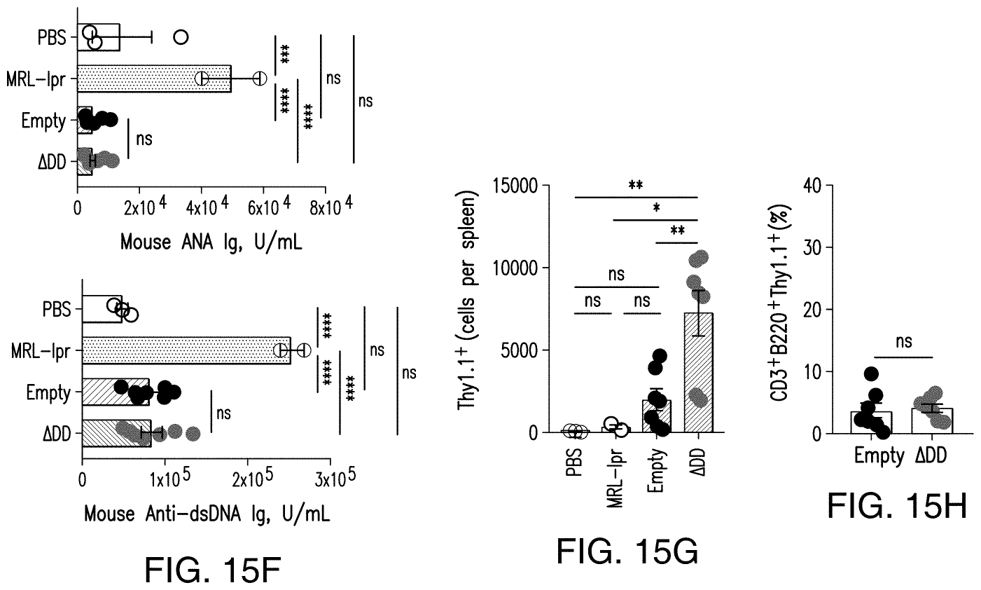

It was previously shown that expression of a transgenic TCR crossed to a Fas-deficient lpr background can limit the development of ALPS (74). Additionally, the B6 strain manifests lymphoproliferative symptoms at a slower rate compared with other strains (49, 50, 75). Therefore. additional experiments to assess the safety of the Fas^{ΔDD} DNR modification were performed by adoptively transferring an open T cell repertoire genetically engineered with either Fas DNR or empty control into the ALPS-susceptible MRL-Mp strain. Fas-deficient mice on an MRL background (MRL-lpr mice) developed auto-antibodies, nephritis, and splenomegaly more severely and many months earlier than B6-lpr mice (FIG. 15A) (49, 50, 75). To induce activation and expansion of adoptively transferred T cells in this model, open-repertoire T cells from the MRL-Mp mouse were co-transduced with a previously described second-generation anti-CD19 2K CAR (71) and the Fas^{ΔDD} or control vector. Use of the anti-CD19 CAR in these experiments promoted strong in vivo proliferation of T cells through recognition of host CD19⁺B cells. Of note, recently published data indicate that T cells modified with a CAR are still able to undergo stimulation through their TCR (72, 76).

The spleens of MRL-Mp mice that received no cells (PBS), or anti-CD19 CAR+ T cells transduced with FasADD or empty control were analyzed and compared with the spleens of age-matched Fas-deficient MRL-lpr mice (FIG. 15C). Spleens from age-matched MRL-lpr mice weighed significantly more when compared with spleens from all other treatment groups. Importantly, no difference was observed in spleen sizes between PBS-treated mice and mice that received anti-CD19 CAR-transduced cells modified either with the Fas^{ΔDD} or control. Flow cytometry analysis of splenocytes demonstrated a robust expansion of unusual DN CD3^{+B}220⁺ lymphocytes in the spleens of MLR-lpr mice that collectively accounted for more than 30% of all lymphocytes (FIGS. 15D and 15E). By contrast, the frequency of CD3^{+B}220⁺lymphocytes in the empty vector and Fas^{ΔDD} T cell-treated mice was similar to levels observed in the PBS control mice.

To assess the development of autoimmunity, serum analysis of all treated animals was performed using samples from MRL-lpr mice as a positive control. Mice that received anti-CD19 CAR′ T cells modified with Fas^{ΔDD} or empty vector had low antinuclear and anti-dsDNA antibody titers comparable to the PBS control (FIG. 15F). In contrast, serum from the MRL-lpr positive control mice demonstrated high titers of both types of autoantibodies. In the absence of uncontrolled lymphoproliferation and the formation of autoantibodies, anti-CD19 CAR′ T cells co-transduced with Fas DNR persisted at significantly higher levels in the spleens of recipient MRL-Mp mice compared with control-modified anti-CD19 CAR′ T cells (FIG. 15G). Further, the persistent Fas DNR-modified CAR′ T cells did not acquire a greater proportion of aberrant CD3^{+B}220⁺ cells compared with control-modified CAR′ cells (FIG. 15H). These results directly mirrored the findings using Fas^{ΔDD}-modified pmel-1 T cells transferred into B6 hosts (FIGS. 4C and 4D).

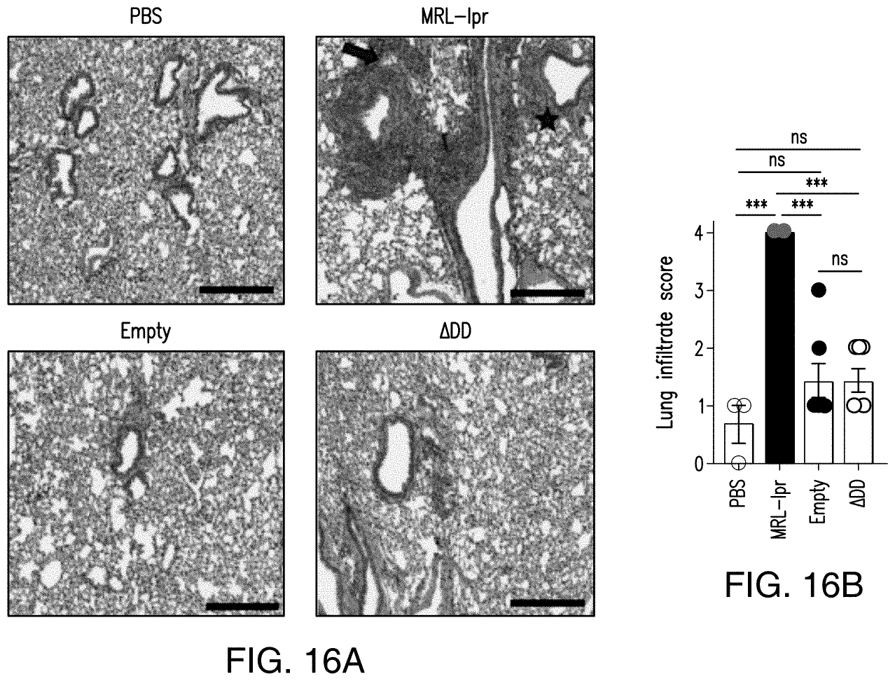

Finally, to assess whether the ALPS-susceptible MRL-Mp recipient mice developed lung pathology following adoptive transfer of Fas DNR-modified T cells, a blinded pathologic assessment of H&E-stained lung specimens was performed. Consistent with previous reports (77), the Fas-deficient MRL-lpr mice developed a dense mononuclear cell inflammatory lung infiltrate in the perivascular and peribronchiolar regions (FIGS. 16A and 16B). By contrast, mice treated with FasΔDD- or control-modified T cells did not display evidence of an increased inflammatory infiltrate relative to PBS-treated control injection. Further, no evidence of pulmonary fibrosis was observed.

Together, these data in both the B6 and MRL-Mp strains demonstrate that despite the augmented relative survival of the Fas^{ΔDD} DNR T cells, no evidence of uncontrolled lymphoaccumulation, formation of a Thy1.1⁺CD3^{+B}220⁺ population, or clinical evidence of autoimmunity was detected. Based on these data, infusion of mature T cells impaired in Fas signaling does not result in an acquired lymphoproliferative phenotype.

T Cell-Intrinsic Disruption of Fas Signaling Enhances Antitumor Efficacy Following ACT

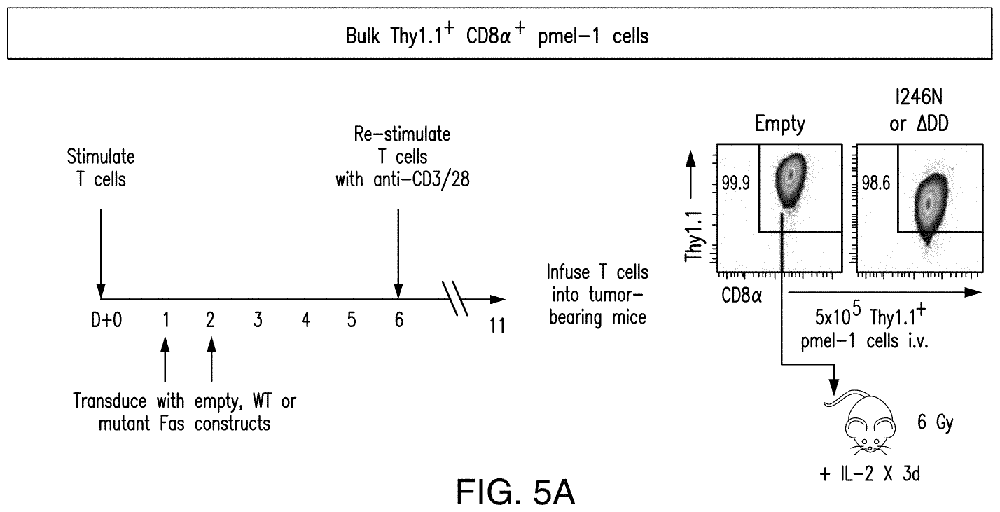

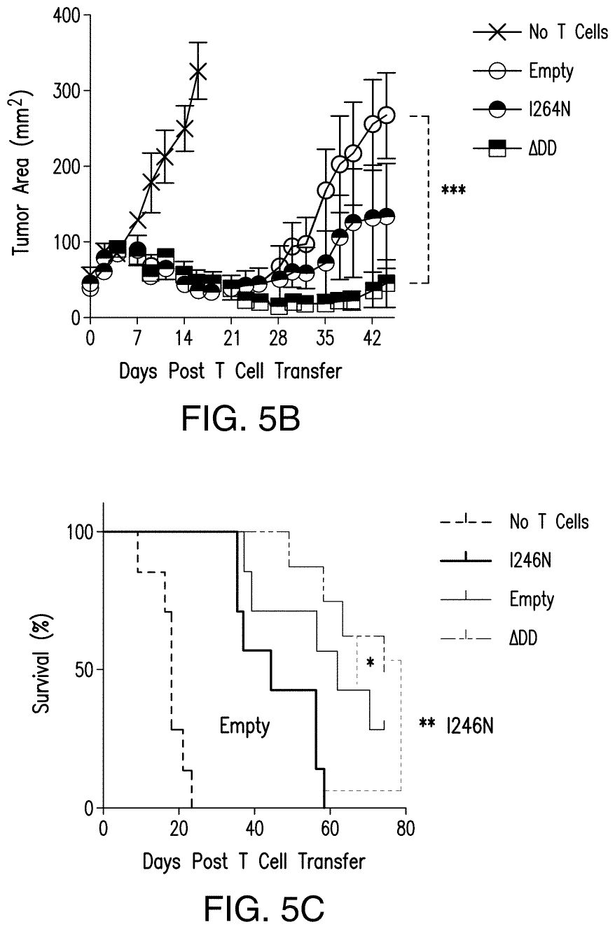

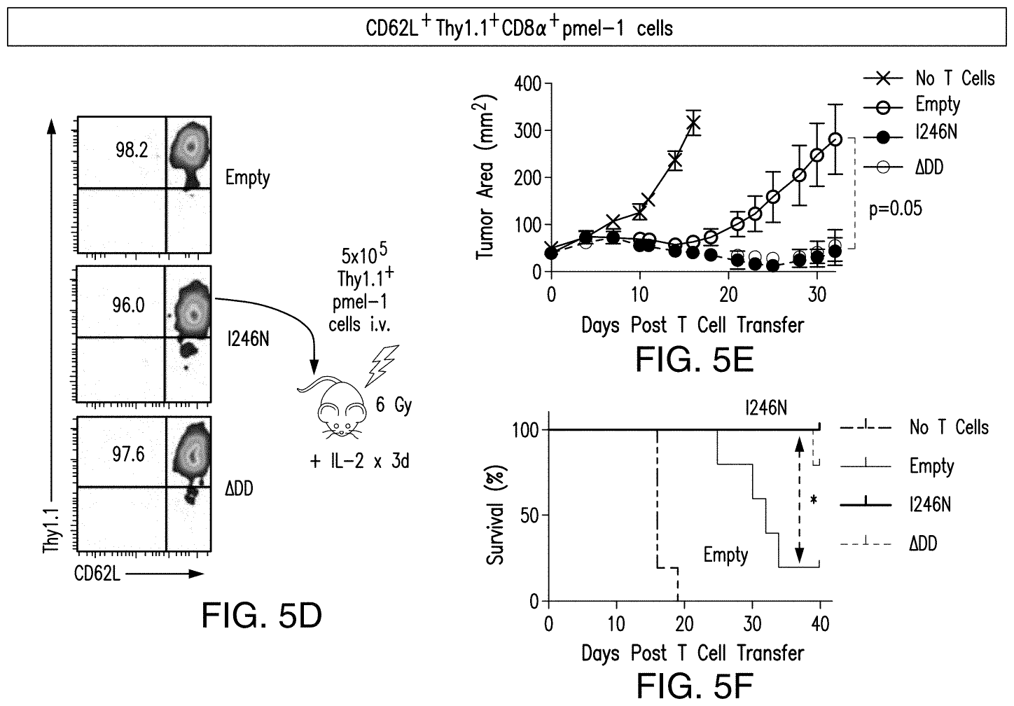

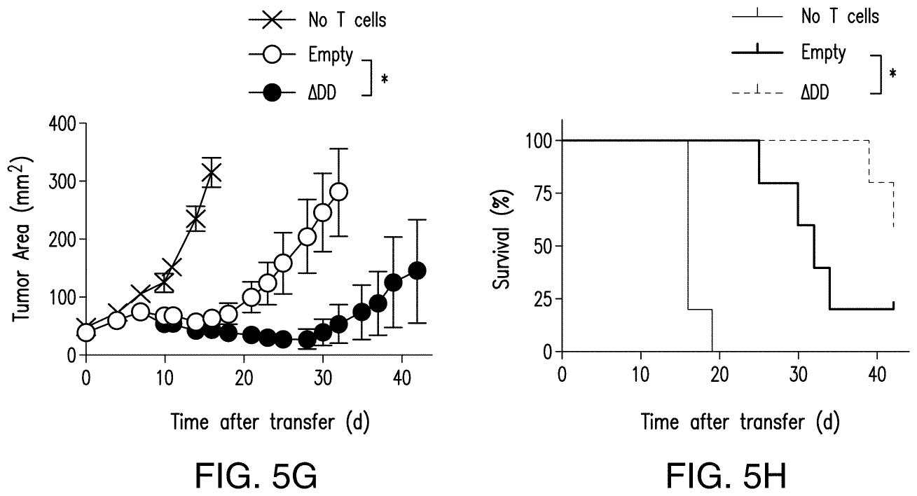

Having established that adoptively transferred T cells engineered with a Fas DNR results in enhanced persistence without long-term toxicity, the antitumor efficacy of these cells was next evaluated. Pmel-1 T cells underwent stimulation and retroviral transduction either with Fas^{I246N}, Fas^{ΔDD}, or an empty vector control. This was followed by re-stimulation and further expansion to mimic the more differentiated T cell populations present in the circulation of cancer patients (5, 31) (FIGS. 1D and 5A). After 11d, transduced T cells from each condition were isolated to >98% purity using anti-Thy1.1 microbeads, then separately injected into sublethally irradiated mice bearing established B16 melanoma tumors. Treated mice also received IL-2 by i.p. injection. Relative to untreated controls, all mice who received adoptively transferred pmel-1 T cells experienced a significant delay in tumor growth (FIG. 5B). However, those mice who received T cells engineered either with the Fas^{I246N} or Fas^{ΔDD} DNRs exhibited enhanced tumor control (FIG. 5B; P<0.001) and significantly improved animal survival relative to control-modified pmel-1 cells (FIG. 5C; P<0.05 and P<0.01).

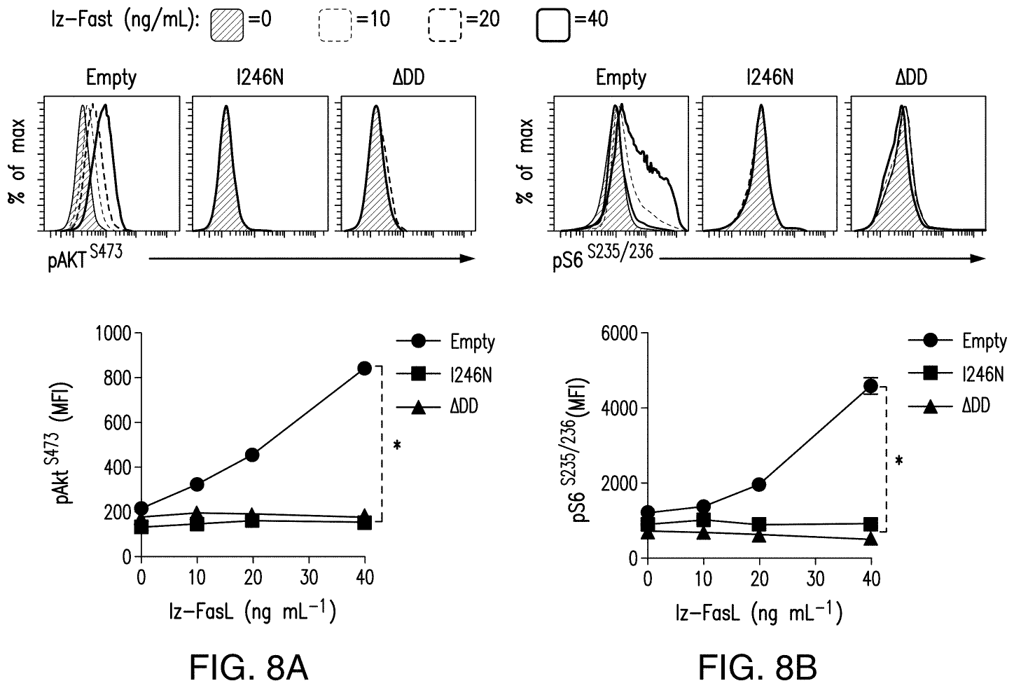

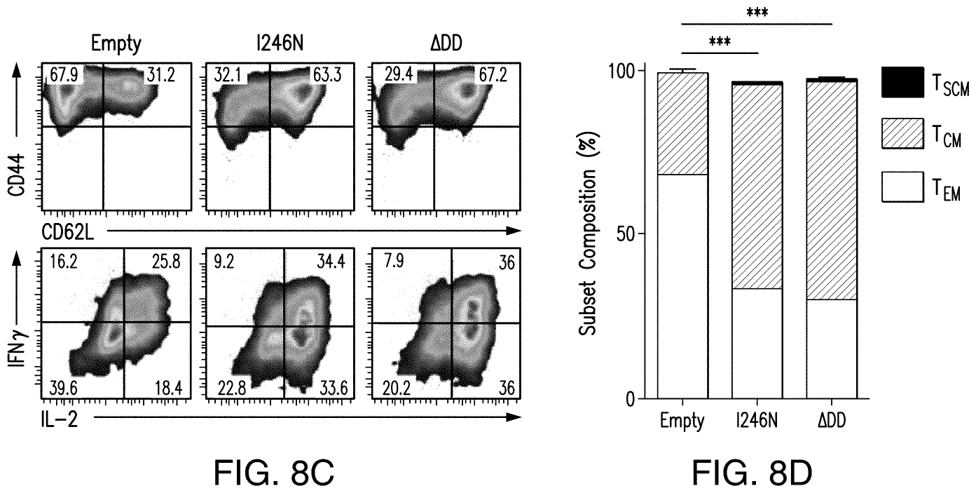

It was recently discovered that Fas stimulation can induce non-apoptotic Akt/mTOR-signaling, resulting in augmented T cell differentiation (51, 52). Consistent with the previous results, it was found that exposure to lz-FasL caused a dose-dependent increase in phosphorylated (p) Akt^{S473} and ^{pS6S235,S236} in T cells transduced with an empty vector control (FIGS. 8A and 8B).

Expansion of control modified cells resulted in an accumulation of TEM-like cells with a reduced capacity to produce IL-2 (FIGS. 8C and 8D). By contrast, T cells transduced with either FasI246N or Fas^{ΔDD} failed to show Akt or S6 phosphorylation and were protected from augmented Akt-mediated T cell differentiation. These cells retained a predominantly TCM-like phenotype and the capacity to produce IL-2. In several different animal models (29, 53, 54) and clinical trials (10, 55), transfer of TCM-like cells was associated with superior tumor regression compared to transfer of TEM-like cells. These findings raised the possibility that the superior tumor regression observed with Fas DNR-modified cells might be attributable to differences in cell differentiation rather than protection from Fas-mediated T cell death. To test this possibility, T cell differentiation status was normalized at the time of cell infusion by isolating to >96% purity transduced, TCM-like phenotype cells (Thy1.1⁺CD44^{hi}g^{h}CD62L⁺) by FACS sorting (FIG. 5D). Central memory-like sorted T cells were subsequently transferred into sublethally irradiated, B16 tumor-bearing mice as described in FIG. 5A. It was found that even when normalized for TCM-like differentiation status, adoptive transfer of T cells modified with the Fas DNRs resulted in superior tumor regression and animal survival compared with control-modified T cells (FIGS. 5E-5H; P<0.05).

Taken together, prevention of Fas-mediated cell death in adoptively transferred, tumor-reactive T cells engineered with a Fas DNR results in superior tumor regression and animal survival.

Genetic Engineering with Fas DNR Protects Human T Cells from Fas-Mediated Apoptosis

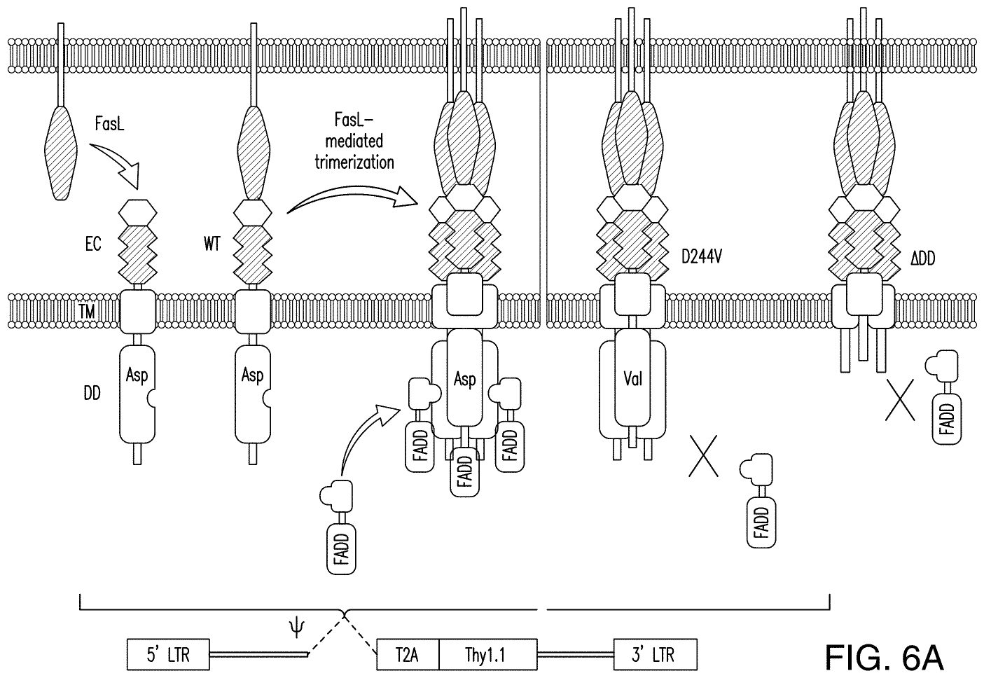

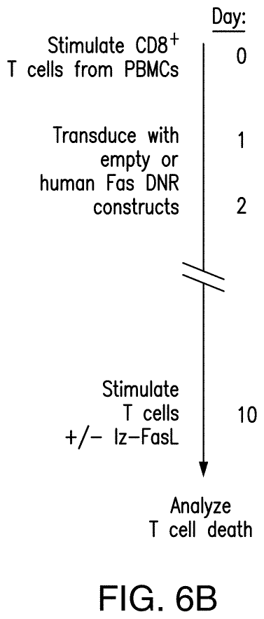

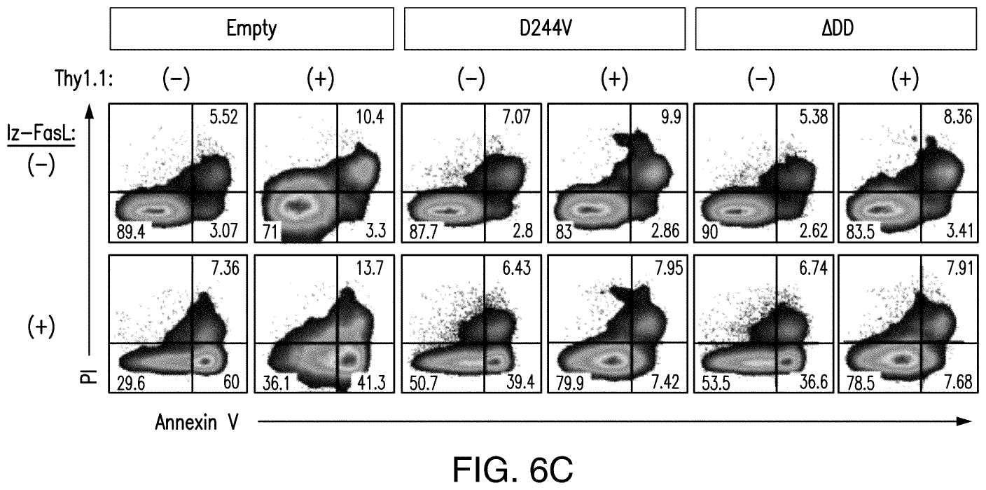

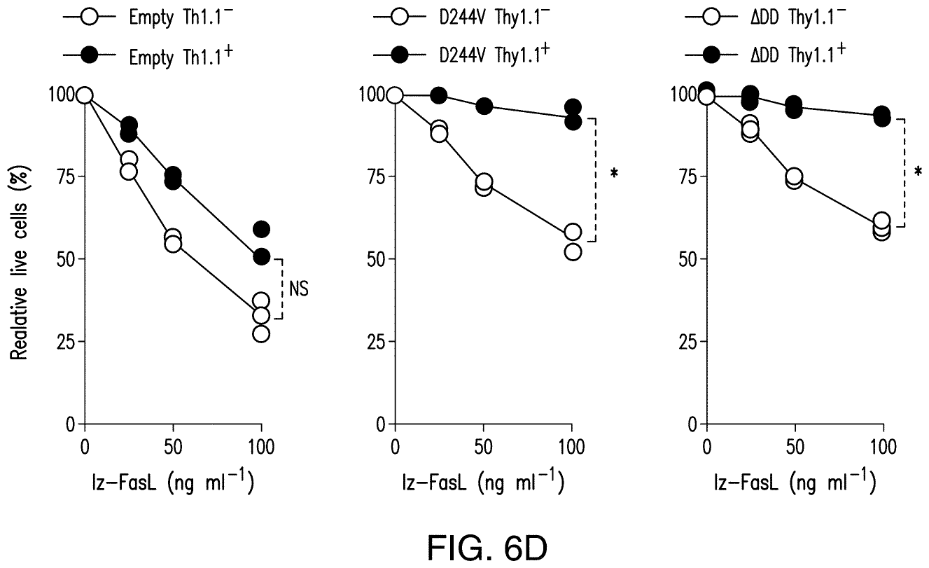

To determine the clinical feasibility of engineering human T cells with Fas DNRs, retroviral constructs encoding the human Fas sequence mutated to prevent FADD binding were designed. This included a human Fas variant containing a point mutation substituting a valine for an aspartate residue at position 244 (hFas^{D244V}) (56, 57), and human Fas with the majority of the intracellular death domain truncated (del aa 230-314; hFas^{ΔDD}) (FIG. 6A) (56, 57).

CD8⁺ T cells were isolated from HD PBMC and stimulated with anti-CD3/CD28 and IL-2, followed by transduction with hFas^{D244V}, hFas^{ΔDD}, or an empty vector control (FIG. 6B). In the absence of additional stimulation, both untransduced Thy1.1⁻ and transduced Thy1.1⁺ T cells remained similarly viable as measured by Annexin V and PI staining (FIG. 6C). However, when these cells were cultured in the presence of increasing doses of lz-FasL, T cells transduced with the empty vector exhibited a significant and dose-dependent increase in the frequency of Annexin apoptotic and necrotic cells (FIGS. 6C and 6D). By contrast, T cells modified with either hFas^{D244V} or hFas^{ΔDD} were significantly protected from lz-FasL-mediated apoptosis. This protection was predominantly T cell-intrinsic, as non-transduced Thy 1. F cells exhibited significantly higher frequency of Annexin V⁺ cells relative to Thy1.1⁺ T cells transduced with hFas^{D244V} or hFas^{ΔDD}. Thus, genetic engineering with a Fas DNR protects primary human T cells from FasL-induced cell death, providing a new method to protect adoptively transferred T cells within the human tumor microenvironment.

Discussion

The results of a pan-cancer analysis here reported strongly suggested that a canonical death-inducing ligand, FASLG, is overexpressed within the majority of human cancer microenvironments. A significant proportion of human T cells used for adoptive immunotherapy co-expressed Fas, the cognate receptor for FasL. Based on these findings, a cell-intrinsic strategy to ‘insulate’ Fas-competent mouse and human T cells from FasL-induced apoptosis using genetic engineering with a series of Fas DNRs was tested. Functionally, adoptively transferred Fas DNR-modified T cells exhibited superior persistence in both the periphery and tumors of tumor-bearing animals, resulting in superior tumor regression and overall survival. Importantly, while T cells modified with Fas DNR exhibited enhanced survival relative to control-modified T cells as late as 6 months following transfer, no evidence of uncontrolled lymphoproliferation or autoimmunity was detected. These findings therefore provide a novel, potentially universal gene engineering strategy to enhance the function of adoptively transferred T cells against a broad range of human malignancies, including advanced solid cancers.

It was previously reported that in addition to its canonical apoptosis-inducing functions, Fas can also promote mouse and human T cell differentiation in an AKT-dependent manner (51, 52). Consistent with these findings, T cells transduced with Fas DNRs were protected from lz-FasL mediated induction of pAKT^{s473} and ^{pS6S235,S236}. Consequently, this block in AKT/mTOR signaling minimized T cell differentiation, promoting the accumulation of TCM-like cells which retained expression of the lymphoid homing marker CD62L and the capacity to produce IL-2. In multiple pre-clinical models (29, 53, 54) and in retrospective analyses of human clinical trials (10, 55), infusion of TCM-like cells was associated with superior antitumor outcomes compared with TEM-like cells. These findings raised the possibility that the superior treatment outcomes using Fas DNR-modified cells might have resulted from the infusion of less differentiated T cells, rather than prevention of apoptosis. To address this possibility, the antitumor efficacy of phenotypically matched, FACS-sorted, Tc_{M}-like cells modified with a Fas DNR or an empty vector control were compared. Even when normalized for surface phenotype, Fas DNR-modified T_{CM} exhibited superior treatment efficacy compared with control-modified T_{CM}. Mechanistically, the dominant contributor of the enhanced in vivo antitumor efficacy using Fas DNR-modified T cells was attributable to the disruption of cell death and not the infusion of less differentiated cells. These findings are also consistent with recent papers from Zhu et al., Horton et al., and Lakins et al. demonstrating that FasL-induced apoptosis of tumor infiltrating lymphocytes limits the efficacy of immune checkpoint inhibitors (17, 58, 59).

While the analyses indicated that FASLG expression is enriched within the microenvironments of many human tumors, they do not define which specific cell type is expressing the ligand. Using immunohistochemical protein staining, previous studies have identified that FasL can be expressed directly on the surface of many of the solid cancers identified in our pan-cancer analysis. This includes cancers of the breast, colon, brain, kidney, and cervix (60, 61). Additionally, recent studies have identified that FasL is expressed along the luminal surface of the neovasculature surrounding human ovarian and brain cancers, creating a tumor endothelial death barrier limiting T cell infiltration (60, 62). Finally, it is possible that FasL can be expressed within the tumor microenvironment by cells of both the innate and adaptive immune system. This possibility has previously been shown by others (17) and is further suggested by our own analysis demonstrating a high degree of correlation between FASLG and many immune-related genes. Finally, the functional data demonstrate that Fas DNR modification also affords protection from other apoptosis-inducing stimuli a T cell might experience following adoptive cell transfer into a tumor- or infection-bearing host. These include activation induced cell death (AICD), cytokine withdrawal, and proximity to antigen-expressing tumor cells. Collectively, these data suggest that the source of FasL is likely to be tumor histology dependent. Thus, a cell-intrinsic Fas DNR approach which does not compromise the FasL-mediated tumor-killing capacity of the transferred T cells is likely to have broad applicability across a range of cancer types.

Fas DNR now joins a list of other candidate DNRs with which a T cell might be modified to intrinsically disrupt signaling by immune-suppressive factors present within the tumor microenvironment, including TGFβ receptor (63) and PD1(64). Disruption of Fas using a short hairpin RNA approach has been reported in human T cells in vitro(65); however, due to the relatively poor efficiency of Fas knock down, this approach required lengthy in vitro selection. Furthermore, the in vivo antitumor capacity of these cells was not tested. Despite observing enhanced cellular persistence using the Fas DNR-modified T cells, evidence of double negative T cell formation or uncontrolled lymphoproliferation was not observed.

Germline loss of function in Fas signaling can result in an auto-immune lymphoproliferative disease in both mice and humans, a potential safety consideration for the Fas DNR approach. Despite augmented survival of FasADD-modified T cells, no evidence of uncontrolled lymphoaccumulation, formation of an aberrant CD3^{+B}220⁺ lymphocyte population, or autoimmunity using 2 different mouse strains was found. This included performing adoptive transfer of a polyclonal T cell population into the ALPS-prone MRL-Mp strain. Based on these data, the infusion of mature T cells impaired in Fas signaling is unlikely to result in an acquired lymphoproliferation syndrome.

Although Fas is a critical mediator for initiating the extrinsic apoptotic signaling cascade, intrinsic apoptotic pathways remain intact in the cells. Thus, competition for homeostatic cytokines, neglect due to an absence of antigen, and T cell exhaustion can all contribute to regulating the homeostasis of the Fas DNR cells in vivo. Despite these reassuring safety data in mice, refinement of this approach for clinical application can include the introduction of a suicide mechanism, such as a truncated EGFR upstream of the Fas DNR (66).

In conclusion, the FasL/Fas pathway is poised to be activated in many patients receiving adoptive immunotherapy for the treatment of solid cancers. Novel dominant negative receptors were developed, which intrinsically abrogate the apoptosis-inducing functions of this pathway in primary mouse and human T cells, leading to enhanced cellular persistence and augmented antitumor efficacy. These data lay the groundwork for a potential universal strategy to enhance the potency of adoptive immunotherapies against both solid and hematologic cancers.

### Example 2—Effects of Fas DNR and Anti-CD19 CAR Modified T Cell Treatment in a Mouse Model of Leukemia

The therapeutic efficacy of adoptively transferred T cells engineered with both a Fas DNR and a CAR was next evaluated. An independent tumor model in which a hematologic malignancy was targeted with a CAR was used. A recently developed syngeneic B cell ALL (B-ALL) line driven by the physiologically relevant E2a-PBX translocation in a treatment model using a murine second-generation 2K anti-CD19 CAR was used (72, 78). A syngeneic model was chosen over the more commonly used xenogeneic anti-CD19 CAR treatment models for two reasons. First, to ensure that the transferred T cells were fully responsive to host-derived FasL in addition to FasL expression by tumor cells and the adoptively transferred T cells. Second, to avoid the potentially confounding influence of xenogeneic reactivity on AICD induction in the transferred T cells.

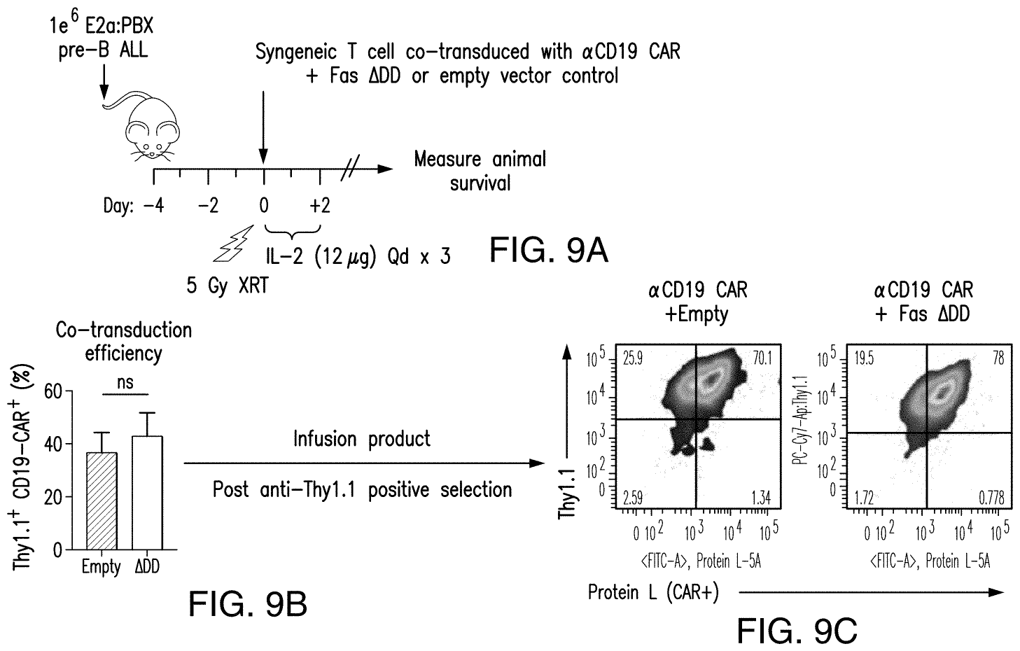

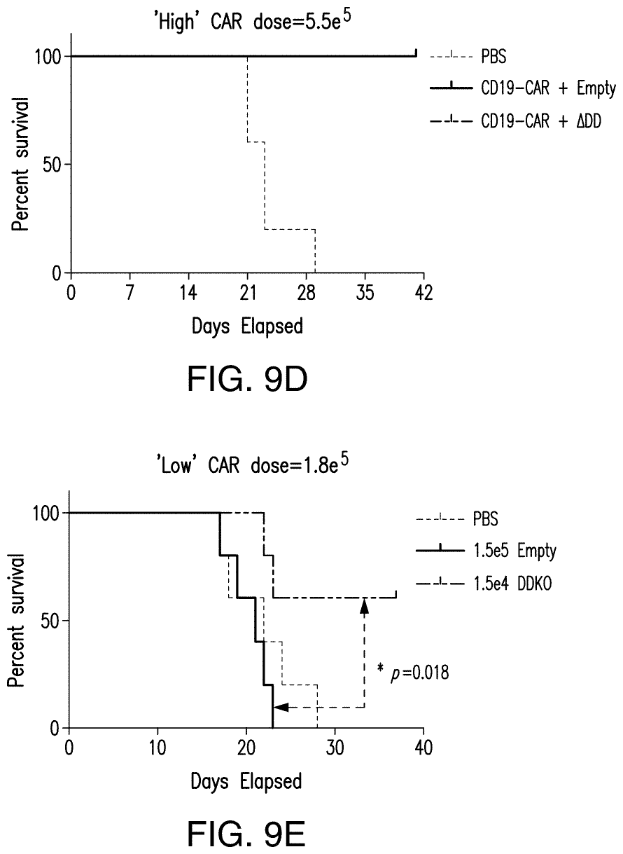

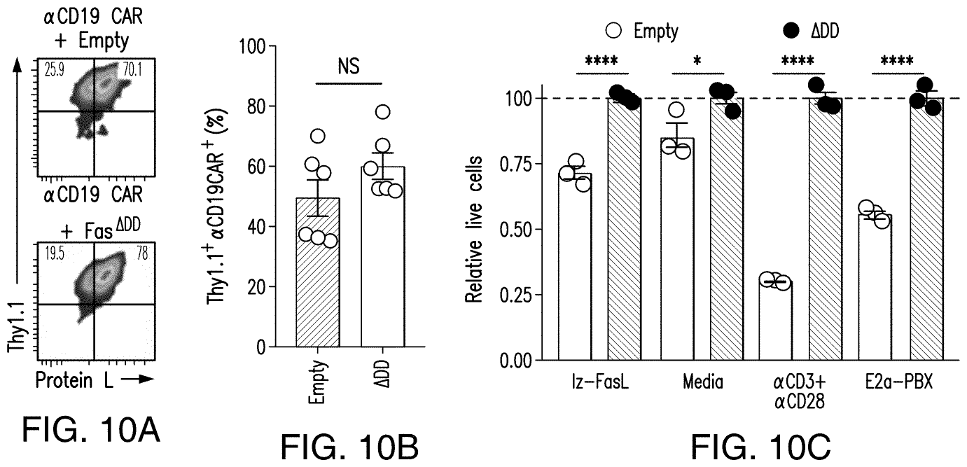

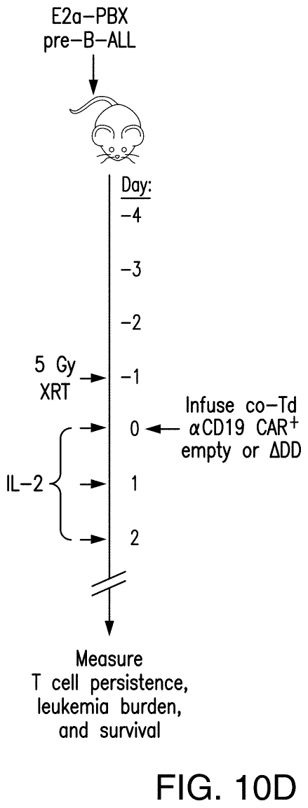

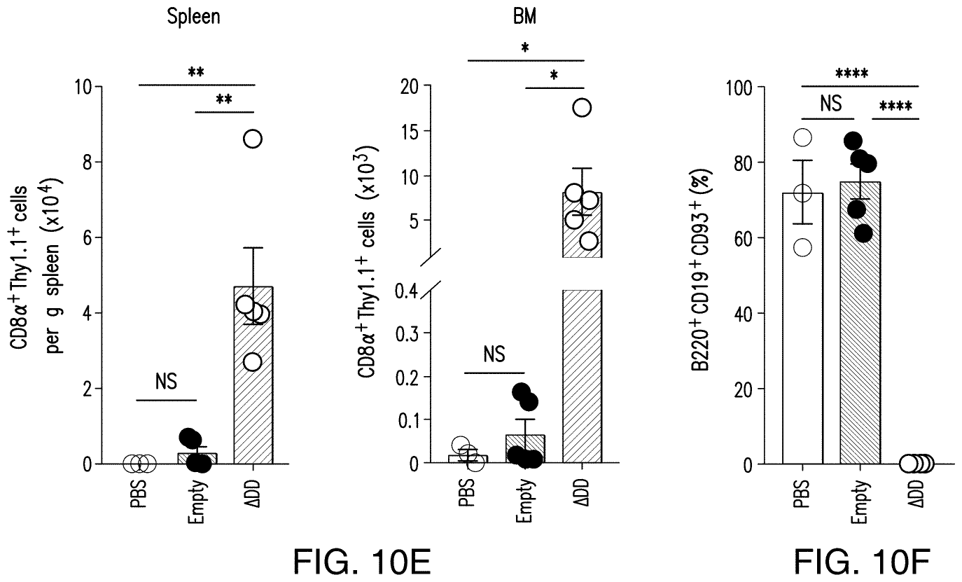

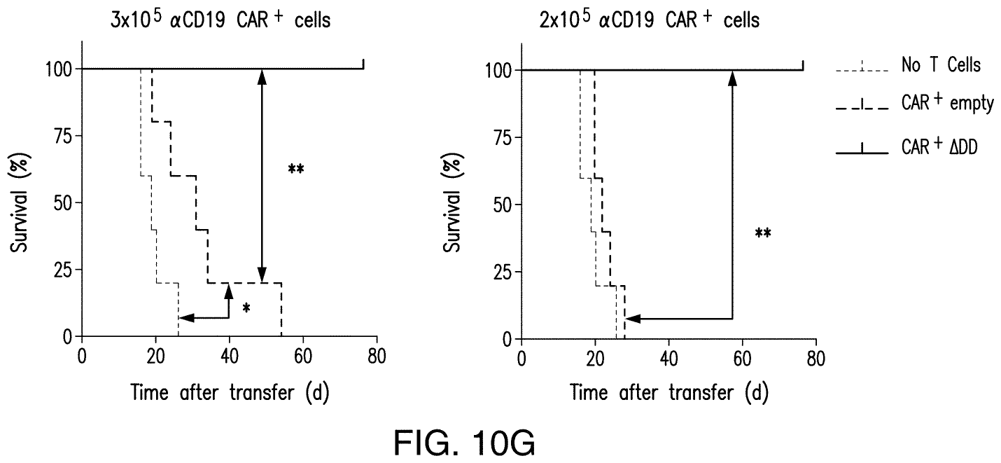

T cells underwent stimulation and retroviral transduction with anti-CD19 CAR and either Fas^{ΔDD} or empty vector control. Co-transduction efficiency and the purity of the transduced T cells are shown in FIGS. 9B-9C and 10A-10B. Using protein L to identify CAR-transduced T cells (79), cotransduction efficiencies were similarly efficient when using Fas^{ΔDD} and the empty vector control following Thy1.1 bead enrichment. Next, how the cotransduced anti-CD19 CAR T cells responded to various apoptosis-inducing stimuli, including exogenous FasL, cytokine withdrawal, AICD, and exposure to antigen-expressing B-ALL tumor cells was determined (FIG. 10C). Similar to the results using TCR-expressing pmel-1 T cells, the expression of Fas^{ΔDD} protected CAR-modified T cells from each of these death-inducing stimuli relative to empty vector control—transduced CAR⁺ T cells.

Experimental design for the treatment with syngeneic T cells co-transduced with anti-CD19 CAR and either Fas^{ΔDD} or empty vector control in a mouse leukemia model is shown in FIGS. 9A and 10D.

Treated mice received daily IL-2 injections for 3 days to support expansion of the adoptively transferred T cells. Fourteen days following cell infusion, the spleens and BM, two disease sites for E2a-PBX B-ALL, were analyzed for persistence of the adoptively transferred cells. Higher levels of Thy1.1+Fas^{ΔDD} cells in both disease sites in comparison to mice that received empty vector-transduced T cells (FIG. 10E) were observed. E2a-PBX leukemia expresses classic pre-B-ALL markers, including CD19, B220, and CD93 (80). As shown in FIG. 10F, the BM in untreated (PBS) and empty vector-treated mice contained roughly 70% leukemia cells 14 days after T cell treatment. However, the mice that received Fas^{ΔDD}-modified cells contained less than 1% leukemia cells in the BM. These data indicated that CAR′ T cells expressing the Fas DNR cells were able to mediate superior leukemia clearance relative to empty vector-transduced T cells.

After 11d, transduced T cells from each condition were isolated to >98% purity using anti-Thy1.1 microbeads, then separately injected into sublethally irradiated mice bearing established E2a:PBX pre-B ALL tumors. Treated mice also received IL-2 by i.p. injection. Relative to untreated controls, all mice who received high dose CAR T cells (5.5×10⁵) experienced a significant delay in tumor growth (FIG. 9D). However, when treated with low dose CART cells (1.8×10⁵), only those mice who received T cells engineered with Fas^{ΔDD} DNR exhibited significantly improved animal survival relative to control (FIG. 9E).

In another experimental setting, the survival of leukemia-bearing mice after adoptive transfer of two different doses of second-generation 2K anti-CD19 CAR-transduced T cells co-modified with Fas^{ΔDD} or empty control was analyzed. In order to provide for a treatment window, doses of CAR-modified T cells previously shown to be subtherapeutic in this model were transferred (72). At a higher cell dose (3×10⁵ CAR⁺ cells), adoptive transfer of either control- or Fas^{ΔDD}-modified CAR′ T cells resulted in significantly improved animal survival compared with mice that did not receive treatment (FIG. 10G, left). However, whereas all mice that received the Fas DNR-modified CAR′ T cells survived, mice that received control-modified CAR′ T cells did not survive longer than 55 days. At a further de-escalated dose of CAR′ cells (2×10⁵), Fas DNR-modified T cells continued to provide long-term survival in 100% of treated mice, while control-modified T cells entirely lost efficacy (FIG. 10G, right). Previous reports have demonstrated that 4-1BB-containing second-generation CARs express higher levels of antiapoptotic proteins compared with CARs containing a CD28 domain (80). These data in the solid cancer B16 melanoma and hematologic E2a-PBX leukemia models indicate that Fas DNR expression in adoptively transferred T cells results in superior in vivo cellular persistence and antitumor efficacy regardless of whether the antigen-targeting structure is a TCR or 2K CAR.

### Example 3—FasDNR Protects Cells from FasL Induced Apoptosis and does not Affect T Cell Tumor-Targeting Functions

Methods

Cell cultures. Platinum-GP retroviral packaging cells (Cell Biolabs) were cultured in RPMI supplied with 10% fetal bovine serum, 10 mM HEPES (Gibco) and 25 Unit/ml PenStrep (Gibco). Primary T cells were cultured in RPMI supplied with 10% heat-inactivated human serum, 25 mM HEPES (Gibco) and 50 Unit/ml PenStrep (Gibco).

Isolation and expansion of human T cells. Buffy coats were acquired from healthy donors at New York Blood Center. Peripheral blood mononuclear cells (PBMC) were isolated by density gradient centrifugation using Lymphocyte Separation Medium (Corning). CD8⁺ T cells were isolated using EasySep Human CD8⁺ T cell Isolation Kit (Stemcell). CD8⁺ T cells were activated on 5 μg/ml anti-CD3 (Miltenyi Biotec) antibody-coated plate and 1 μg/ml soluble anti-CD28 (Miltenyi Biotec). For viral transduction, T cells were treated with 50 IU/ml of IL-2 (PeproTech) for 2 days prior to transduction.

Plasmid design and viral transduction. All plasmids for viral packaging were designed based on SFGγ retroviral vector. A feline endogenous retrovirus envelope RD114 was used for co-transfection with SFGy vector in Platinum-GP cell. Lipofectamine 3000 (ThermoFisher) was used for Platinum-GP cell co-transfection. Primary T cells were transduced with viral supernatant on Retronectin (Takara) coated plate. Briefly, plate was coated with 20 ug/ml Retronectin at 4° C. overnight then blocked by PBS with 2% FBS for 30 min at room temperature. Plate was washed with PBS and loaded with viral supernatant. Centrifugation was done at 2000 g, 32° C. for 2 hr. Supernatant was aspirated and cells were loaded into each well. Plate was centrifuged again at 1200 rpm, 32° C. for 5 min and incubated at 37° C. for 2 days.

Flow cytometry and intracellular staining. Conjugated antibodies used for flowcytometry includes Brilliant Violet 421TM anti-human EGFR (AY13, Biolegend), PE/Cy5 anti-human CD95 Fas (DX2, Biolegend), APC/Cyanine7 anti-human CD95 Fas (DX2, Biolegend), PerCP/Cyanine5.5 anti-human TNF-α (Mab 11, Biolegend). For NY-ESO targeting TCR, PE anti-TCR Vβ13.1 222, Beckman Coulter) was used. For CAR staining, an Alexa Fluor 647 AffiniPure F(ab′) 2 Fragment Goat Anti-Mouse IgG, F(ab′) 2 antibody (Jackson ImmunoResearch) was used.

FasL apoptosis assay. A form of soluble FasL oligomerized through a leucine zipper motif (FasL-LZ) was used at 100 ng/ml for all apoptosis assays. Cells were treated with FasL-LZ at deisgned time points at 37° C. Cells were washed and stained for surface antibodies. Cells were stained with CellEvent™ Caspase-3/7 Green Detection Reagent (ThermoFisher) in FACS buffer for 25 min at 37° C. and washed twice. Cells were then stained with APC Annexin V (Biolegend) in Annexin V Binding Buffer (Biolegend) for 25 min at room temperature. Cells were washed twice and resuspended in Annexin V Binding Buffer for flowcytometry.

Statistical analysis. All statistical analyses were performed using the Prism 7 (GraphPad) software. No statistical methods were used to predetermine sample sizes. All analysis was done on triplicated samples. Statistical comparisons between two groups were calculated by paired Student's t-tests for matched samples. P<0.05 is considered statically important.

Results

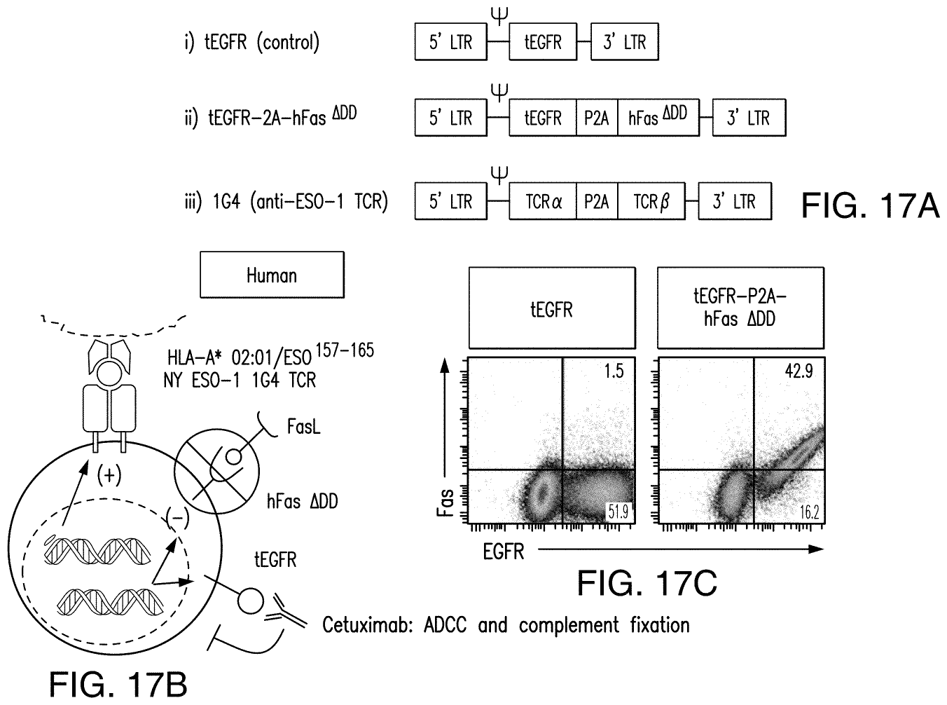

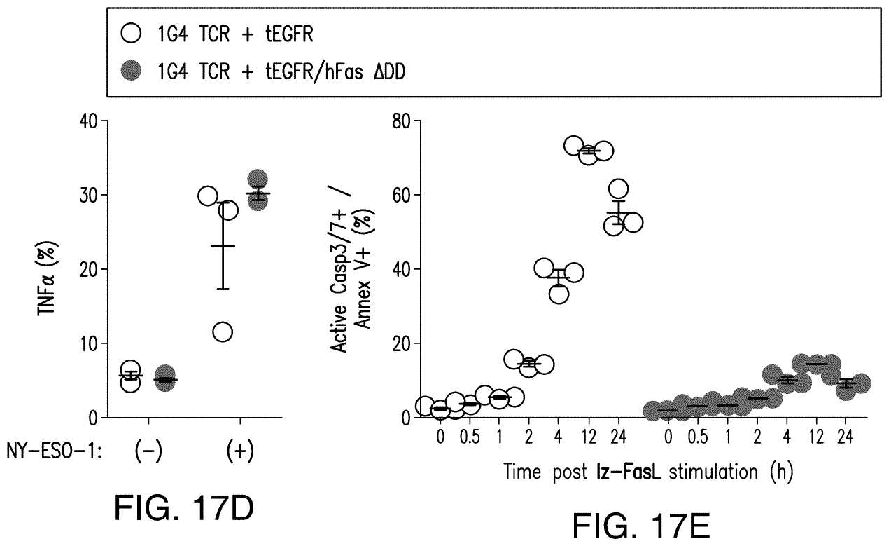

The functionality of T cells engineered with a Fas DNR and an antigen-recognizing receptor (both a TCR and a CAR) was also evaluated. Multiple constructs were designed as shown in FIG. 17A. The resulting engineered human primary T cells expressed a Fas^{DNR} protecting T cells from FasL-induced apoptosis, a T cell receptor (TCR) targeting the NY-ESO1 antigen, and an EGFRt that can be targeted by monoclonal antibodies to induce antibody-dependent cell-mediated cytotoxicity (ADCC) or complement-depentent cytotoxicity (FIG. 17B). Cells expressed Fas^{DNR} and tEGFR. After antigen stimulation, both control and FasDNR cells showed increased TNFa staining (FIG. 17D). Furthermore, after exposure to FasL leucine zipper (FasL-1z) at different time points, the T cells expressing Fas^{DNR} showed reduced staining to apoptotic markers.

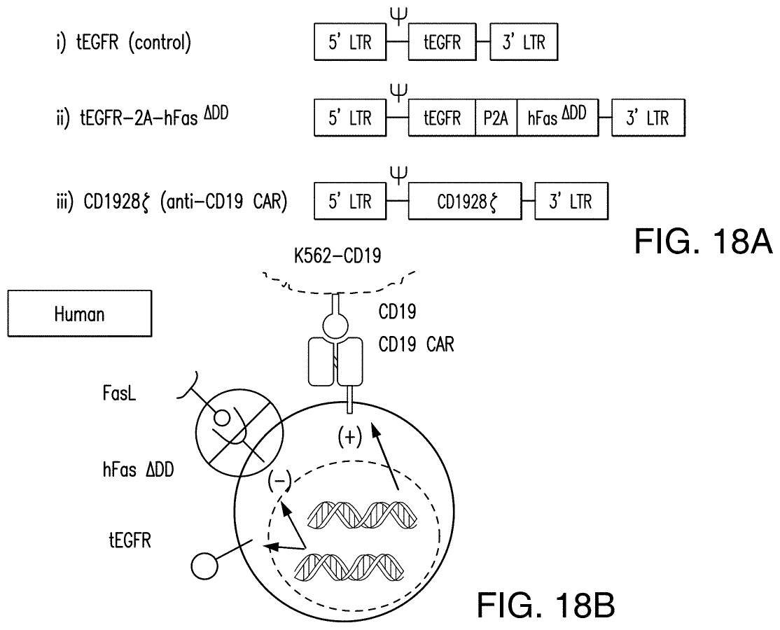

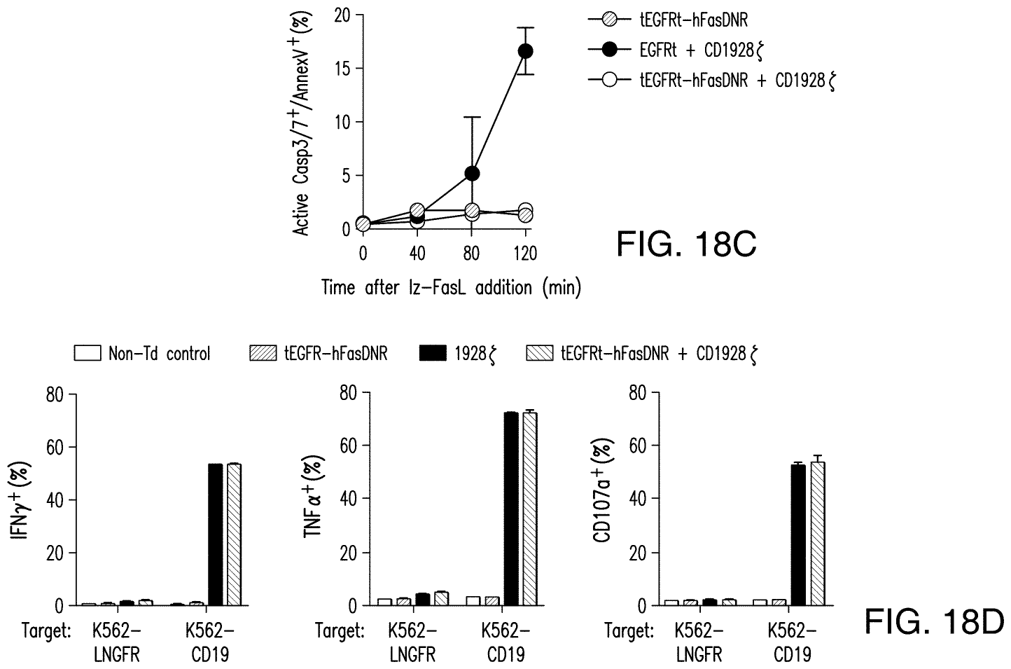

Similarly, the functionality of T cells was evaluated after co-engineering of primary human T cells with a Fas^{DNR}, a trackable truncated EGFR, and an antigen-specific CAR anti-CD19 (CD192ζ) (FIGS. 18A and 18B). After exposure to FasL leucine zipper (1z-FasL), T cells expressing the Fas^{DNR} were protected by apoptosis, independently of the expression of the anti-CD19 CAR (FIG. 18C). Furthermore, after co-incubation with K562 cells expressing CD19, T cells expressing anti-CD19 CAR alone (19280 or in combination with Fas^{DNR} (tEGFR-hFASDNR+CD1928ζ) showed comparable antigen-specific cytokine release and degranulation (FIG. 18D). Thus, Fas^{DNR} reduces the apoptosis induced by FasL without altering the T cell functions.

### REFERENCES

1. S. L. Maude et al., Tisagenlecleucel in Children and Young Adults with B-Cell Lymphoblastic Leukemia. N Engl J Med 378, 439-448 (2018).
2. S. S. Neelapu et al., Axicabtagene Ciloleucel CAR T-Cell Therapy in Refractory Large B-Cell Lymphoma. N Engl J Med 377, 2531-2544 (2017).
3. D. W. Lee et al., T cells expressing CD19 chimeric antigen receptors for acute lymphoblastic leukaemia in children and young adults: a phase 1 dose-escalation trial. Lancet 385, 517-528 (2015).
4. D. L. Porter et al., Chimeric antigen receptor T cells persist and induce sustained remissions in relapsed refractory chronic lymphocytic leukemia. Sci Transl Med 7, 303ra139 (2015).
5. C. J. Turtle et al., Immunotherapy of non-Hodgkin's lymphoma with a defined ratio of CD8+ and CD4+CD19-specific chimeric antigen receptor-modified T cells. Sci Transl Med 8, 355ra1 16 (2016).
6. C. A. Ramos et al., Clinical and immunological responses after CD30-specific chimeric antigen receptor-redirected lymphocytes. J Clin Invest 127, 3462-3471 (2017).
7. J. H. Park et al., Long-Term Follow-up of CD19 CAR Therapy in Acute Lymphoblastic Leukemia. N Engl J Med 378, 449-459 (2018).
8. T. J. Fry et al., CD22-targeted CART cells induce remission in B-ALL that is naive or resistant to CD19-targeted CAR immunotherapy. Nat Med, (2017).
9. R. L. Siegel, K. D. Miller, A. Jemal, Cancer statistics, 2018. CA Cancer J Clin 68, 7-30 (2018).
10. C. U. Louis et al., Antitumor activity and long-term fate of chimeric antigen receptor-positive T cells in patients with neuroblastoma. Blood 118, 6050-6056 (2011).
11. M. R. Parkhurst et al., T cells targeting carcinoembryonic antigen can mediate regression of metastatic colorectal cancer but induce severe transient colitis. Mol Ther 19, 620-626 (2011).
12. N. Ahmed et al., Human Epidermal Growth Factor Receptor 2 (HER2)-Specific Chimeric Antigen Receptor-Modified T Cells for the Immunotherapy of HER2-Positive Sarcoma. J Clin Oncol 33, 1688-1696 (2015).
13. Y. C. Lu et al., Treatment of Patients With Metastatic Cancer Using a Major Histocompatibility Complex Class II-Restricted T-Cell Receptor Targeting the Cancer Germline Antigen MAGE-A3. J Clin Oncol 35, 3322-3329 (2017).
14. C. A. Klebanoff, S. A. Rosenberg, N. P. Restifo, Prospects for gene-engineered T cell immunotherapy for solid cancers. Nat Med 22, 26-36 (2016).
15. M. V. Maus, C. H. June, Making Better Chimeric Antigen Receptors for Adoptive T-cell Therapy. Clin Cancer Res 22, 1875-1884 (2016).
16. L. Gattinoni, C. A. Klebanoff, N. P. Restifo, Paths to stemness: building the ultimate antitumour T cell. Nat Rev Cancer 12, 671-684 (2012).
17. J. Zhu et al., Resistance to cancer immunotherapy mediated by apoptosis of tumor-infiltrating lymphocytes. Nat Commun 8, 1404 (2017).
18. S. A. Rosenberg et al., Durable complete responses in heavily pretreated patients with metastatic melanoma using T-cell transfer immunotherapy. Clin Cancer Res 17, 45504557 (2011).
19. S. Stevanovic et al., Landscape of immunogenic tumor antigens in successful immunotherapy of virally induced epithelial cancer. Science 356, 200-205 (2017).
20. A. Heczey et al., CAR T Cells Administered in Combination with Lymphodepletion and PD-1 Inhibition to Patients with Neuroblastoma. Mol Ther 25, 2214-2224 (2017).
21. E. A. Chong et al., PD-1 blockade modulates chimeric antigen receptor (CAR)-modified T cells: refueling the CAR. Blood 129, 1039-1041 (2017).
22. G. P. Adams et al., High affinity restricts the localization and tumor penetration of single-chain fv antibody molecules. Cancer Res 61, 4750-4755 (2001).
23. M. A. Postow, R. Sidlow, M. D. Hellmann, Immune-Related Adverse Events Associated with Immune Checkpoint Blockade. N Engl J Med 378, 158-168 (2018).
24. D. Aran et al., Comprehensive analysis of normal adjacent to tumor transcriptomes. Nat Commun 8, 1077 (2017).
25. G. T. Consortium, The Genotype-Tissue Expression (GTEx) project. Nat Genet 45, 580585 (2013).
26. B. Li, C. N. Dewey, RSEM: accurate transcript quantification from RNA-Seq data with or without a reference genome. BMC Bioinformatics 12, 323 (2011).
27. A. Subramanian et al., Gene set enrichment analysis: a knowledge-based approach for interpreting genome-wide expression profiles. Proc Natl Acad Sci USA 102, 15545-15550 (2005).
28. D. Hamann et al., Phenotypic and functional separation of memory and effector human CD8+ T cells. J Exp Med 186, 1407-1418 (1997).
29. L. Gattinoni et al., A human memory T cell subset with stem cell-like properties. Nat Med 17, 1290-1297 (2011).
30. K. E. Pauken et al., Epigenetic stability of exhausted T cells limits durability of reinvigoration by PD-1 blockade. Science 354, 1160-1165 (2016).
31. C. L. Mackall et al., Distinctions between CD8+ and CD4+ T-cell regenerative pathways result in prolonged T-cell subset imbalance after intensive chemotherapy. Blood 89, 3700-3707 (1997).
32. D. Kagi et al., Fas and perforin pathways as major mechanisms of T cell-mediated cytotoxicity. Science 265, 528-530 (1994).
33. Z. Hao, T. W. Mak, Type I and type II pathways of Fas-mediated apoptosis are differentially controlled by XIAP. J Mot Cell Biol 2, 63-64 (2010).
34. A. S. Mohamood et al., Protection from autoimmune diabetes and T-cell lymphoproliferation induced by FasL mutation are differentially regulated and can be uncoupled pharmacologically. Am J Pathol 171, 97-106 (2007).
35. R. M. Siegel et al., Fas preassociation required for apoptosis signaling and dominant inhibition by pathogenic mutations. Science 288, 2354-2357 (2000).
36. M. P. Boldin et al., A novel protein that interacts with the death domain of Fas/APO1 contains a sequence motif related to the death domain. J Biol Chem 270, 7795-7798 (1995).
37. A. M. Chinnaiyan, K. O'Rourke, M. Tewari, V. M. Dixit, FADD, a novel death domain-containing protein, interacts with the death domain of Fas and initiates apoptosis. Cell 81, 505-512 (1995).
38. J. P. Medema et al., FLICE is activated by association with the CD95 death-inducing signaling complex (DISC). EMBO J 16, 2794-2804 (1997).
39. F. C. Kischkel et al., Cytotoxicity-dependent APO-1 (Fas/CD95)-associated proteins form a death-inducing signaling complex (DISC) with the receptor. EMBO J 14, 55795588 (1995).
40. C. H. June, B. R. Blazar, J. L. Riley, Engineering lymphocyte subsets: tools, trials and tribulations. Nat Rev Immunol 9, 704-716 (2009).
41. R. Watanabe-Fukunaga, C. I. Brannan, N. G. Copeland, N. A. Jenkins, S. Nagata, Lymphoproliferation disorder in mice explained by defects in Fas antigen that mediates apoptosis. Nature 356, 314-317 (1992).
42. M. Eberstadt, B. Huang, E. T. Olejniczak, S. W. Fesik, The lymphoproliferation mutation in Fas locally unfolds the Fas death domain. Nat Struct Biol 4, 983-985 (1997).
43. M. Ramaswamy et al., Specific elimination of effector memory CD4+ T cells due to enhanced Fas signaling complex formation and association with lipid raft microdomains. Cell Death Differ 18, 712-720 (2011).
44. J. R. Veatch et al., Tumor infiltrating BRAFV600E-specific CD4 T cells correlated with complete clinical response in melanoma. J Clin Invest, (2018).
45. S. S. Chandran et al., Treatment of metastatic uveal melanoma with adoptive transfer of tumour-infiltrating lymphocytes: a single-centre, two-stage, single-arm, phase 2 study. Lancet Oncol 18, 792-802 (2017).
46. A. G. Chapuis et al., T-Cell Therapy Using Interleukin-21-Primed Cytotoxic T-Cell Lymphocytes Combined With Cytotoxic T-Cell Lymphocyte Antigen-4 Blockade Results in Long-Term Cell Persistence and Durable Tumor Regression. J Clin Oncol 34, 37873795 (2016).
47. P. L. Cohen, R. A. Eisenberg, Lpr and gld: single gene models of systemic autoimmunity and lymphoproliferative disease. Annu Rev Immunol 9, 243-269 (1991).
48. V. K. Rao, J. B. Oliveira, How I treat autoimmune lymphoproliferative syndrome. Blood 118, 5741-5751 (2011).
49. H. C. Morse, 3rd et al., Abnormalities induced by the mutant gene Ipr: expansion of a unique lymphocyte subset. J Immunol 129, 2612-2615 (1982).
50. S. Izui et al., Induction of various autoantibodies by mutant gene lpr in several strains of mice. J Immunol 133, 227-233 (1984).
51. C. A. Klebanoff et al., Memory T cell-driven differentiation of naive cells impairs adoptive immunotherapy. J Clin Invest 126, 318-334 (2016).
52. A. C. Cruz et al., Fas/CD95 prevents autoimmunity independently of lipid raft localization and efficient apoptosis induction. Nat Commun 7, 13895 (2016).
53. C. A. Klebanoff et al., Central memory self/tumor-reactive CD8+ T cells confer superior antitumor immunity compared with effector memory T cells. Proc Natl Acad Sci USA 102, 9571-9576 (2005).
54. D. Sommermeyer et al., Chimeric antigen receptor-modified T cells derived from defined CD8+ and CD4+ subsets confer superior antitumor reactivity in vivo. Leukemia 30, 492500 (2016).
55. J. N. Kochenderfer et al., Lymphoma Remissions Caused by Anti-CD19 Chimeric Antigen Receptor T Cells Are Associated With High Serum Interleukin-15 Levels. J Clin Oncol 35, 1803-1813 (2017).
56. D. A. Martin et al., Defective CD95/APO-1/Fas signal complex formation in the human autoimmune lymphoproliferative syndrome, type Ia. Proc Natl Acad Sci USA 96, 4552-4557 (1999).
57. C. E. Jackson et al., Autoimmune lymphoproliferative syndrome with defective Fas: genotype influences penetrance. Am J Hum Genet 64, 1002-1014 (1999).
58. B. L. Horton, J. B. Williams, A. Cabanov, S. Spranger, T. F. Gajewski, Intratumoral CD8(+) T-cell Apoptosis Is a Major Component of T-cell Dysfunction and Impedes Antitumor Immunity. Cancer Immunol Res 6, 14-24 (2018).
59. M. A. Lakins, E. Ghorani, H. Munir, C. P. Martins, J. D. Shields, Cancer-associated fibroblasts induce antigen-specific deletion of CD8 (+) T Cells to protect tumour cells. Nat Commun 9, 948 (2018).
60. S. Kleber et al., Yes and PI3K bind CD95 to signal invasion of glioblastoma. Cancer Cell 13, 235-248 (2008).
61. M. E. Peter et al., The role of CD95 and CD95 ligand in cancer. Cell Death Differ 22, 549-559 (2015).
62. G. T. Motz et al., Tumor endothelium FasL establishes a selective immune barrier promoting tolerance in tumors. Nat Med 20, 607-615 (2014).
63. C. M. Bollard et al., Tumor-Specific T-Cells Engineered to Overcome Tumor Immune Evasion Induce Clinical Responses in Patients With Relapsed Hodgkin Lymphoma. J Clin Oncol, JCO2017743179 (2018).
64. L. Cherkassky et al., Human CAR T cells with cell-intrinsic PD-1 checkpoint blockade resist tumor-mediated inhibition. J Clin Invest 126, 3130-3144 (2016).
65. G. Dotti et al., Human cytotoxic T lymphocytes with reduced sensitivity to Fas-induced apoptosis. Blood 105, 4677-4684 (2005).
66. X. Wang et al., A transgene-encoded cell surface polypeptide for selection, in vivo tracking, and ablation of engineered cells. Blood 118, 1255-1263 (2011).
67. W. W. Overwijk et al., Tumor regression and autoimmunity after reversal of a functionally tolerant state of self-reactive CD8+ T cells. J Exp Med 198, 569-580 (2003).
68. S. P. Kerkar et al., Genetic engineering of murine CD8+ and CD4+ T cells for preclinical adoptive immunotherapy studies. J Immunother 34, 343-352 (2011).
69. P. F. Robbins et al., Tumor regression in patients with metastatic synovial cell sarcoma and melanoma using genetically engineered lymphocytes reactive with NY-ESO-1. J Clin Oncol 29, 917-924 (2011).
70. R. Eil et al., Ionic immune suppression within the tumour microenvironment limits T cell effector function. Nature 537, 539-543 (2016).
71. J. N. Kochendelfer et al., Adoptive transfer of syngeneic T cells transduced with a chimeric antigen receptor that recognizes murine CD19 can eradicate lymphoma and normal B cells. Blood. 2010; 116(19):3875-3886.
72. Y. Yang et al., TCR engagement negatively affect CD8 but not CD4 CAR T cell expansion and leukemic clearance. Sci Transl Med. 2017; 9(417):eaag1209.
73. H. Z. Imtiyaz et al., Structural requirements for signal-induced target binding of FADD determined by functional reconstitution of FADD deficiency. J Biol Chem. 2005; 280(36):31360-31367.
74. J. D. Mountz et al., Defective clonal deletion and anergy induction in TCR transgenic lpr/lpr mice. Semin Immunol. 1994; 6(1):27-37.
75. G. G. Singer et al., Apoptosis, Fas and systemic autoimmunity: the MRL-lpr/lpr model. Curre Opin Immunol. 1994; 6(6):913-920.
76. C. Y. Slaney et al., Dual-specific chimeric antigen receptor T cells and an indirect vaccine eradicate a variety of large solid tumors in an immune-competent, self-antigen setting. Clin Cancer Res. 2017; 23(10):2478-2490.
77. Z. Hao et al., T cell-specific ablation of Fas leads to Fas ligand-mediated lymphocyte depletion and inflammatory pulmonary fibrosis. J Exp Med. 2004; 199(10):1355-1365.
78. E. Jacoby et al., Murine allogeneic CD19 CAR T cells harbor potent antileukemic activity but have the potential to mediate lethal GVHD. Blood. 2016; 127(10):1361-1370.
79. Z. Zheng et al., Protein L: a novel reagent for the detection of chimeric antigen receptor (CAR) expression by flow cytometry. J Transl Med. 2012; 10-29.
80. G. Li et al., 4-1BB enhancement of CART function requires NF-κB and TRAFs. JCI Insight. 2018; 3(18):121322.

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A cell comprising:
(a) an antigen-recognizing receptor that binds to an antigen, and
(b) an exogenous dominant negative Fas polypeptide.

2. The cell of claim 1, wherein the dominant negative Fas polypeptide comprises at least one modification in a cytoplasmic death domain of human Fas.

3. The cell of claim 2, wherein the at least one modification is selected from the group consisting of mutations, deletions, and insertions.

4. The cell of claim 3, wherein the mutation is a point mutation.

5. The cell of claim 2, wherein the at least one modification in the cytoplasmic death domain prevents the binding between the dominant negative Fas polypeptide and a FADD polypeptide.

6. The cell of claim 1, wherein the dominant negative Fas polypeptide comprises a deletion of amino acids 230-314 of a human Fas consisting of the amino acid sequence set forth in SEQ ID NO: 10.

7. The cell of claim 6, wherein the dominant negative Fas polypeptide comprises a) an amino acid sequence that is at least about 80% identical to the amino acid sequence set forth in SEQ ID NO: 12; or b) the amino acid sequence set forth in SEQ ID NO: 12.

8. The cell of claim 1, wherein the dominant negative Fas polypeptide comprises a point mutation at position 260 of a human Fas consisting of the amino acid sequence set forth in SEQ ID NO: 10.

9. The cell of claim 8, wherein the point mutation is D260V.

10. The cell of claim 8, wherein the dominant negative Fas polypeptide comprises a) an amino acid sequence that is at least about 80% identical to the amino acid sequence set forth in SEQ ID NO: 14; or b) the amino acid sequence set forth in SEQ ID NO: 14.

11. The cell of claim 1, wherein the exogenous dominant negative Fas polypeptide enhances cell persistence of the immunoresponsive cell, and/or reduces apoptosis or anergy of the.

12. The cell of claim 1, wherein the antigen-recognizing receptor is recombinantly expressed and/or expressed from a vector.

13. The cell of claim 1, wherein the exogenous dominant negative Fas polypeptide is expressed from a vector.

14. The cell of claim 1, wherein the cell is an immunoresponsive cell.

15. The cell of claim 1, wherein the cell is a cell of the lymphoid lineage or a cell of the myeloid lineage.

16. The cell of claim 1, wherein the cell is selected from the group consisting of a T cell, a Natural Killer (NK) cell, a B cell, a monocyte and a macrophage.

17. The cell of claim 1, wherein the cell is a T cell.

18. The cell of claim 17, wherein the T cell is a cytotoxic T lymphocyte (CTL), a regulatory T cell, or a Natural Killer T (NKT) cell.

19. The cell of claim 1, wherein the antigen is a tumor antigen or a pathogen antigen.

20. The cell of claim 1, wherein the antigen is a tumor antigen.

21. The cell of claim 20, wherein the tumor antigen is selected from the group consisting of CD19, MUC16, MUC1, CAIX, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-α2, κ-light chain, KDR, mutant KRAS, mutant PIK3CA, mutant IDH, mutant p53, mutant NRAS, LeY, L1 cell adhesion molecule, MAGE-A1, Mesothelin, ERBB2, MAGEA3, CT83 (also known as KK-LC-1), p53, MART1, GP100, Proteinase3 (PR1), Tyrosinase, Survivin, hTERT, EphA2, NKG2D ligands, NY-ES0-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, PRAIVIE, HPV E6 oncoprotein, HPV E7 oncoprotein, and ERBB.

22. The cell of claim 21, wherein the antigen is CD19.

23. The cell of claim 1, wherein the antigen is a pathogen-associated antigen.

24. The cell of claim 23, wherein the pathogen-associated antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

25. The cell of claim 1, wherein the antigen-recognizing receptor is a T cell receptor (TCR) or a chimeric antigen receptor (CAR).

26. The cell of claim 25, wherein the TCR is a) an endogenous TCR that recognizes a pathogen-associated antigen, and the cell is a pathogen-specific T cell; or b) an endogenous TCR that recognizes a tumor antigen, and the cell is a tumor-specific T cell.

27. The cell of claim 25, wherein the CAR comprises an extracellular antigen-binding domain, a transmembrane domain, and an intracellular signaling domain.

28. The cell of claim 27, wherein the intracellular signaling domain further comprises at least one co-stimulatory signaling region.

29. The cell of claim 28, wherein the at least one co-stimulatory signaling region comprises a CD28 polypeptide.

30. The cell of claim 1, further comprising a suicide gene.

31. The cell of claim 30, wherein the suicide gene is a Herpes simplex virus thymidine kinase (hsv-tk), inducible Caspase 9 Suicide gene (iCasp-9) or a truncated human epidermal growth factor receptor (EGFRt) polypeptide.

32. A composition comprising an effective amount of a cell of claim 1.

33. The composition of claim 32, wherein the composition is the pharmaceutical composition that further comprises a pharmaceutically acceptable excipient.

34. A method of inducing and/or enhancing an immune response to a target antigen, reducing tumor burden in a subject, treating and/or preventing a neoplasia, lengthening survival of a subject having a neoplasia, treating blood cancer in a subject, treating a solid tumor in a subject, and/or preventing and/or treating a pathogen infection, the method comprising administering to the subject an effective amount of the cells of claim 1.

35. A method for producing an antigen-specific cell, the method comprising introducing into a cell (a) a first nucleic acid encoding an antigen-recognizing receptor that binds to an antigen; and (b) a second nucleic acid encoding an exogenous dominant negative Fas polypeptide.

36. A nucleic acid composition comprising (a) a first nucleic acid encoding an antigen-recognizing receptor and (b) a second nucleic acid encoding an exogenous dominant negative Fas polypeptide.

37. A vector comprising the nucleic acid composition of claim 36.

38. A kit comprising a cell of claim 1.
